(19) United States
(12) Patent Application Publication
(10) Pub. No.: US 2006/0165664 A1
(43) Pub. Date: Jul. 27, 2006

(54) Method of inducing an enhanced immune response against hiv

(76) Inventors: Emilio A. Emini, Wayne, PA (US); John W Shiver, Chalfont, PA (US); Danilo R Casimiro, Harleysville, PA (US); Andrew J Bett, Lansdale, PA (US); Xiaoping Liang, Eagleville, PA (US); Tong-Ming Fu, Ambler, PA (US)

Correspondence Address: MERCK AND CO., INC, P O BOX 2000, RAHWAY, NJ, 07065-0907, US

(21) Appl. No.: 10/507,237

(22) PCT Filed: Mar. 12, 2003
(86) PCT No.: PCT/US03/07727

§ 371 Date: Sep. 9, 2004

Related U.S. Application Data
(60) Provisional application No. 60/363,807, filed on Mar. 13, 2002.

(51) Int. Cl. A61K 48/00; A61K 39/21

(52) U.S. Cl. 424/93.21; 424/208.1

(57) ABSTRACT

An efficient means of inducing an immune response against human immunodeficiency virus (HIV) utilizing specific prime-boost regimes is disclosed. The specific prime-boost regimes employ a heterologous prime-boost protocol employing recombinant adenoviral vectors of alternative and distinct serotypes comprising exogenous genetic material encoding a common HIV antigen. Vaccines administered into living vertebrate tissue in accordance with the disclosed regimes, preferably a mammalian host, such as a human or a non-human mammal of commercial or domestic veterinary importance, express the HIV-1 antigen (e.g., Gag), inducing a cellular immune response which specifically recognizes HIV-1. It is believed that the disclosed prime/boost regime will offer a prophylactic advantage to previously uninfected individuals and/or provide a therapeutic effect by reducing viral load levels within an infected individual, thus prolonging the asymptomatic phase of HIV-1 infection.

### CROSS-REFERENCE TO RELATED APPLICATIONS

The present application claims priority to provisional application U.S. Ser. No. 60/363,807, filed Mar. 13, 2002, hereby incorporated by reference herein.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to an enhanced means for inducing an immune response against human immunodeficiency virus (“HIV”). Recombinant adenovirus vehicles comprising exogenous genetic material encoding a common HIV antigen are employed in a heterologous prime-boost administration. More particularly, recombinant adenovirus vehicles of alternative and distinct serotypes are employed in heterologous prime-boost immunization schemes. Applicants have found that administration of a recombinant adenoviral vehicle comprising exogenous genetic material encoding an HIV antigen followed by subsequent administration of a recombinant adenovirus of a different serotype comprising the antigen notably amplifies the immune response from the initial administration(s). This amplification is, further, notably higher than that observed upon utilizing the same respective recombinant adenoviral vectors independently for both priming and boosting administrations of mammalian hosts. The amplified immune response which is particularly manifest in the cellular immune response is, further, capable of specifically recognizing HIV. Viruses of use in the instant invention can be any replication-defective adenovirus, provided that the adenovirus of choice is capable of effecting expression of exogenous genetic material incorporated into the viral sequence. Based on the findings disclosed herein, it is believed that the disclosed prime/boost regime will offer a prophylactic advantage to previously uninfected individuals and/or provide a therapeutic effect by reducing viral load levels within an infected individual, thus prolonging the asymptomatic phase of HIV-1 infection.

### BACKGROUND OF THE INVENTION

Human Immunodeficiency Virus-1 (HIV-1) is the etiological agent of acquired human immune deficiency syndrome (AIDS) and related disorders. HIV-1 is an RNA virus of the Retroviridae family and exhibits the 5′ LTR-gag-pol-eniv-LTR 3′ organization of all retroviruses. The integrated form of HIV-1, known as the provirus, is approximately 9.8 Kb in length. Each end of the viral genome contains flanking sequences known as long terminal repeats (LTRs). The HIV genes encode at least nine proteins and are divided into three classes; the major structural proteins (Gag, Pol, and Env), the regulatory proteins (Tat and Rev); and the accessory proteins (Vpu, Vpr, Vif and Nef).

Effective treatment regimes for HIV-1 infected individuals have become available. However, these drugs will not have a significant impact on the disease in many parts of the world and they will have a minimal impact in halting the spread of infection within the human population. As is true of many other infectious diseases, a significant epidemiologic impact on the spread of HIV-1 infection will only occur subsequent to the development and introduction of an effective vaccine. There are a number of factors that have contributed to the lack of successful vaccine development to date. For instance, it is now apparent that in a chronically infected person there exists constant virus production in spite of the presence of anti-HIV-1 humoral and cellular immune responses and destruction of virally infected cells. As in the case of other infectious diseases, the outcome of disease is the result of a balance between the kinetics and the magnitude of the immune response and the pathogen replicative rate and accessibility to the immune response. Pre-existing immunity may be more successful with an acute infection than an evolving immune response can be with an established infection. A second factor is the considerable genetic variability of the virus. Although anti-HIV-1 antibodies exist that can neutralize HIV-1 infectivity in cell culture, these antibodies are generally virus isolate-specific in their activity. It has proven impossible to define serological groupings of HIV-1 using traditional methods. Rather, the virus seems to define a serological “continuum” so that individual neutralizing antibody responses, at best, are effective against only a handful of viral variants. Given this latter observation, it would be useful to identify immunogens and related delivery technologies that are likely to elicit anti-HIV-1 cellular immune responses. It is known that in order to generate CTL responses antigen must be synthesized within or introduced into cells, subsequently processed into small peptides by the proteasome complex, and translocated into the endoplasmic reticulum/Golgi complex secretory pathway for eventual association with major histocompatibility complex (MHC) class I proteins. CD8⁺ T lymphocytes recognize antigen in association with class I MUC via the T cell receptor (TCR) and the CD8 cell surface protein. Activation of naive CD8⁺ T cells into activated effector or memory cells generally requires both TCR engagement of antigen as described above as well as engagement of costimulatory proteins. Optimal induction of CTL responses usually requires “help” in the form of cytokines from CD4⁺ T lymphocytes which recognize antigen associated with MHC class II molecules via TCR and CD4 engagement.

Adenoviral vectors have been developed as live viral vectors for the delivery and expression of various foreign antigens including HIV and have proven to be effective in eliciting a significant CTL response in treated individuals. Adenoviruses are non-enveloped viruses containing a linear double-stranded genome of about 36 kb.

The vectors achieve high viral titres, have a broad cell tropism, and can infect nondividing cells. Adenoviral vectors are very efficient gene transfer vehicles and are frequently used in clinical gene therapy studies. In addition, adenovirus has formed the basis of many promising viral immunization protocols.

European Patent Applications 0 638 316 (Published Feb. 15, 1995) and 0 586 076 (Published Mar. 9, 1994), (both assigned to American Home Products Corporation) describe replicating adenovirus vectors carrying an HIV gene, including env or gag. Various treatment regimes based on these vectors were used with chimpanzees and dogs, some of which included booster adenovirus or protein plus alum treatments.

Replication-defective adenoviral vectors harboring deletions, for instance, in the E1 region constitute a safer alternative to their replicating counterparts. Recent adenoviral vectors have incorporated the known packaging repeats into these vectors; e.g., see EP 0 707 071, disclosing, inter alia, an adenoviral vector deleted of E1 sequences from base pairs 459 to 3328; and U.S. Pat. No. 6,033,908, disclosing, inter alia, an adenoviral vector deleted of base pairs 459-3510. The packaging efficiency of adenovirus has been taught to depend on the number of incorporated individual A (packaging) repeats; see, e.g., Gräble and Hearing, 1990 J. Virol. 64(5):2047-2056; Gräble and Hearing, 1992 J. Virol. 66(2):723-731.

Adenovirus serotypes 5 and 6 have been disclosed and are publicly available (see, American Type Culture Collection (“ATCC”) Accession Deposit Nos. VR-5 and VR-6; respectively). The wildtype adenovirus serotype 5 sequence is, further, known and described in the art; see, Chroboczek et al., 1992 J. Virology 186:280-5. The complete sequence for adenovirus serotype 6, which is provided in FIGS. 11A-1 to 11A-14, was first disclosed in copending U.S. Provisional Application Ser. No. 60/328,655, filed on Oct. 11, 2001. Adenovirus serotype 6, as serotype 5, has been described previously in the literature; see Rowe et al., 1953 Proc. Soc. Exp. Biol. Med. 84:570; Rowe et al., 1955 Am. J. Hyg. 61:197-218; and Hierholzer et al., 1991 Arch. Virol. 121:179-97. Adenovirus serotypes other than Ad5 and Ad6 are also known and described in the literature.

Administration protocols employing viral vaccine vectors to date have employed various prime-boost inoculation schemes. Two general schemes frequently used are: (1) wherein both priming and boosting of the mammalian host is accomplished using the same virus vehicle, and (2) wherein the priming and boosting is carried out utilizing different vehicles not necessarily limited to virus vehicles. Examples of the latter are, for instance, a scheme composed of a DNA prime and viral boost, and one composed of a viral prime and a viral boost wherein alternate virus are used.

It would be of great import in the battle against AIDS to develop a prophylactic- and/or therapeutic-based HIV vaccine strategy capable of generating a strong cellular immune response against HIV infection. The present invention addresses and meets these needs by disclosing a heterologous prime-boost HIV immunization regime based on the administration of recombinant adenoviral vectors of alternative and distinct serotypes, wherein the recombinant adenoviral vectors comprise exogenous genetic material encoding a common HIV antigen. One aspect of the instant invention concerns heterologous immunization schemes employing recombinant adenoviral vectors derived from adenovirus serotypes 5, 6, and 35. A vaccine protocol in accords with this description, as far as Applicants are aware, has not been demonstrated for HIV. This vaccine prime-boost regime may be administered to a host, such as a human.

### SUMMARY OF THE INVENTION

The present invention relates to an enhanced method for generating an immune response against human immunodeficiency virus (“HIV”). The method is based on the heterologous prime-boost administration of recombinant adenovirus vehicles of alternative and distinct serotypes comprising heterologous genetic material encoding an HIV antigen to effect a more pronounced immune response against HIV than that which can be obtained by either vector independently in a single modality prime-boost immunization scheme. In accordance with the disclosed methods, a mammalian host is first administered a priming dose comprising a recombinant adenoviral vector of a first serotype comprising a gene encoding an HIV antigen and, after a period of time, administered a boosting dose comprising a recombinant adenoviral vector of a second and different serotype carrying the gene encoding the HIV antigen. There may be a predetermined minimum amount of time separating the administrations, which time essentially allows for an immunological rest. In particular embodiments, this rest is for a period of at least 4 months. Multiple primings typically, 1-4, are usually employed, although more may be used. The length of time between priming and boost may typically vary from about four months to a year, but other time frames may be used. Applicants have found that boosting of the adenovirus-primed response with an adenovirus of an alternative and distinct serotype leads to a notably amplified immune response to the HIV antigen. Thus the instant invention relates to the administration of alternate serotype adenovirus HIV vaccines in accordance with the disclosed methods.

Accordingly, the instant invention relates to a method for inducing an enhanced immunological response against an HIV-1 antigen in a mammalian host comprising the steps of (a) inoculating the mammalian host with a recombinant adenoviral vector of a first serotype which is at least partially deleted in E1 and devoid of E1 activity comprising a gene encoding an HIV-1 antigen or an immunologically relevant modification thereof; and thereafter (b) inoculating the mammalian host with a boosting immunization comprising a recombinant adenoviral vector of a second and different serotype at least partially deleted in E1 and devoid of E1 activity comprising a gene encoding an HIV-1 antigen or immunologically relevant modification thereof.

The recombinant adenoviral vectors used in the immunization regimes of the present invention may comprise any replication-defective adenoviral vector which is genetically stable through large-scale production and purification of the virus. In other words, a recombinant adenoviral vector suitable for use in the methods of the instant invention can be any purified recombinant replication-defective virus shown to be genetically stable through multiple passages in cell culture which remains so during large-scale production and purification procedures. Such a recombinant virus vector and harvested virus vaccine lends itself to large scale dose filling and subsequent worldwide distribution procedures which will be demanded of an efficacious monovalent or multivalent HIV vaccine. The present invention meets this basic requirement with description of an immunization regime which is based on the use of recombinant replication-defective adenovirus serotypes examples but not limitations of which include serotypes 5, 6, and 35.

Adenoviral vectors preferred for use in the immunization regimes of the instant invention are those that are at least partially deleted in E1 and devoid of E1 activity. Vectors in accordance with this description can be readily propagated in E1-complementing cell lines, such as PER.C6® cells.

The recombinant adenoviral vectors of use in the instant application whether intended as the priming or boosting vehicle must comprise a gene encoding an HIV antigen. In specific embodiments, the gene encoding the HIV antigen or immunologically relevant modification thereof comprises codons optimized for expression in a mammalian host (e.g., a human). Recombinant adenoviral vectors of use in the methods of the instant invention can comprise a gene expression cassette comprising (a) nucleic acid encoding an HIV antigen (e.g., an HIV protein) or biologically active and/or immunologically relevant portion thereof; (b) a heterologous (non-native) or modified native promoter operatively linked to the nucleic acid of part a); and, (c) a transcription termination sequence. A heterologous promoter can be any promoter under the sun (modified or not) which is not native to, or derived from, the virus in which it will be used.

HIV antigens of use in the instant invention include the various HIV proteins, immunologically relevant modifications, and immunogenic portions thereof. The present invention, thus, encompasses the various forms of codon-optimized HIV-1 gag (including but by no means limited to p55 versions of codon-optimized full length (“FL”) Gag and tPA-Gag fusion proteins), HIV-1 pol, HIV-1 nef, HIV-1 env, fusions of the above constructs, and selected modifications of the above possessing immunological relevance. Examples of HIV-1 Gag, Pol, Env, and/or Nef fusion proteins include but are not limited to fusion of a leader or signal peptide at the NH₂-terminal portion of the viral antigen coding region. Such a leader peptide includes but is not limited to a tPA leader peptide.

Recombinant viral vectors in accordance with the instant disclosure form an aspect of the instant invention. Other aspects of the instant invention are host cells comprising said adenoviral vectors; vaccine compositions comprising said vectors; and methods of producing the vectors comprising (a) introducing the adenoviral vector into a host cell which expresses adenoviral E1 protein, and (b) harvesting the resultant adenoviral vectors.

The present invention also relates to prime-boost regimes wherein the recombinant adenoviral vectors comprise various combination of the above HIV antigens. Such HIV immunization regimes will provide for an enhanced cellular immune response subsequent to host administration, particularly given the genetic diversity of human MHCs and of circulating virus. Examples, but not limitations, include viral vector-based multivalent vaccine compositions which provide for a divalent (e.g., gag and nef, gag and pol, or pol and nef components) or a trivalent vaccine (e.g., gag, pol and nef components) composition. Such a multivalent vaccine may be filled for a single dose or may consist of multiple inoculations of each individually filled component. To this end, preferred vaccine compositions of use in the methods of the instant application are recombinant adenovirus vectors comprising multiple, distinct V antigen classes. Each HIV antigen class is subject to sequence manipulation, thus providing for a multitude of potential vaccine combinations; and such combinations are within the scope of the present invention. The utilization of such combined modalities increase the probability of eliciting an even more potent cellular immune response when compared to inoculation with a single modality regime.

The concept of a “combined modality” as disclosed herein also covers the alternative mode of administration whereby multiple HIV-1 viral antigens may be ligated into a proper shuttle plasmid for generation of a recombinant viral vector comprising multiple open reading frames. For example, a trivalent vector may comprise a gag-pol-nef fusion, or possibly a “2+1” divalent vaccine comprising, for instance, a gag-pol fusion (i.e., codon optimized p55 gag and inactivated optimized pol) within the same backbone, with each open reading frame being operatively linked to a distinct promoter and transcription termination sequence. Alternatively, the two open reading frames may be operatively linked to a single promoter, with the open reading frames operatively linked by an internal ribosome entry sequence (IRES).

Administration of the recombinant adenoviral vectors via the disclosed heterologous means provides for improved cellular-mediated immune responses; responses more pronounced than that afforded by single modality regimes. An effect of the improved vaccine should be a lower transmission rate to previously uninfected individuals (i.e., prophylactic applications) and/or reduction in the levels of the viral loads within an infected individual (i.e., therapeutic applications), so as to prolong the asymptomatic phase of HIV-1 infection. The administration, intracellular delivery and expression of the vaccine in this manner elicits a host CTL and Th response. The individual vaccinee or mammalian host (as referred to herein) can be a primate (both human and non-human) as well as any non-human mammal of commercial or domestic veterinary importance.

In light hereof, the present invention relates to methodology regarding administration of the recombinant adenoviral HIV vaccines to provide effective immunoprophylaxis, to prevent establishment of an HIV-1 infection following exposure to this virus, or as a post-HIV infection therapeutic vaccine to mitigate the acute HIV-1 infection so as to result in the establishment of a lower virus load with beneficial long term consequences. Such treatment regimes may include a monovalent or multivalent composition, and/or various combined modality applications. Therefore, the present invention provides for methods of using the disclosed HIV vaccine administration scheme within the various parameters disclosed herein as well as any additional parameters known in the art which, upon introduction into mammalian tissue, induces intracellular expression of the HIV antigen(s) and an effective immune response to the respective HIV antigen(s).

To this end, the present invention relates in part to methods of generating a cellular immune response in a vaccinee, preferably a human vaccinee, wherein the individual is given the recombinant adenovirus HIV vaccines in the manner described.

As used throughout the specification and claims, the following definitions and abbreviations are used:

“HAART” refers to—highly active antiretroviral therapy—.

“first generation” vectors are characterized as being replication-defective. They typically have a deleted or inactivated E1 gene region, and often have a deleted or inactivated E3 gene region as well.

“AEX” refers to Anion Exchange chromatography.

“QPA” refers to Quick PCR-based Potency Assay.

“bps” refers to base pairs.

“s” or “str” denotes that the transgene is in the E1 parallel or “straight” orientation.

“PBMCs” refers to peripheral blood monocyte cells.

“FL” refers to full length.

“FLgag” refers to a full-length optimized gag gene, as shown in FIG. 2.

“Ad5-Flgag” refers to an adenovirus serotype 5 replication-deficient virus which carries an expression cassette which comprises a full length optimized gag gene under the control of a CMV promoter.

“Promoter” means a recognition site on a DNA strand to which an RNA polymerase binds. The promoter forms an initiation complex with RNA polymerase to initiate and drive transcriptional activity. The complex can be modified by activating sequences such as enhancers or inhibiting sequences such as silencers.

“Leader” means a DNA sequence at the 5′ end of a structural gene which is transcribed along with the gene. This usually results in a protein having an N-terminal peptide extension, often referred to as a pro-sequence.

“Intron” means a section of DNA occurring in the middle of a gene which does not code for an amino acid in the gene product. The precursor RNA of the intron is excised and therefore not transcribed into mRNA or translated into protein.

“Immunologically relevant” or “biologically active,” when used in the context of a viral protein, means that the protein is capable, upon administration, of eliciting a measurable immune response within an individual sufficient to retard the propagation and/or spread of the virus and/or to reduce the viral load present within the individual. The same terms, when used in the context of a nucleotide sequence, means that the sequence is capable of encoding for a protein capable of the above.

“Cassette” refers to a nucleic acid sequence which is to be expressed, along with its transcription and translational control sequences. By changing the cassette, a vector can express a different sequence.

“bGHpA” refers to a bovine growth hormone transcription terminator/polyadenylation sequence.

“tPAgag” refers to a fusion between the tissue plasminogen activator leader sequence and an optimized HIV gag gene.

Where utilized, “IA” or “inact” refers to an inactivated version of a gene (e.g. IApol).

“MCS” is “multiple cloning site”.

“Ad5” is adenovirus of serotype 5.

“Ad6” is adenovirus of serotype 6.

In general, adenoviral constructs, gene constructs are named by reference to the genes contained therein. For example:

“Ad5 HIV-1 gag”, also referred to as the original HIV-1 gag adenoviral vector, is a vector containing a transgene cassette composed of a hCMV intron A promoter, the full length version of the human codon-optimized HIV-1 gag gene, and the bovine growth hormone polyadenylation signal.

“MRK Ad5 HIV-1 gag” also referred to as “MRKAd5gag” or “Ad5gag2” is an adenoviral vector which is deleted of E1, and contains adenoviral base pairs 1-450 and 3511-3523, with a human codon-optimized HIV-1 gag gene in an E1 parallel orientation under the control of a CMV promoter without intron A. The construct also comprises a bovine growth hormone polyadenylation signal.

“pV1JnsHIVgag”, also referred to as “HIVFLgagPR9901”, is a plasmid comprising the CMV immediate-early (IE) promoter and intron A, a full-length codon-optimized HIV gag gene, a bovine growth hormone-derived polyadenylation and transcriptional termination sequence, and a minimal pUC backbone.

“pV1JnsCMV(no intron)-FLgag-bGHpA” is a plasmid derived from pV1JnsHIVgag which is deleted of the intron A portion of CMV and which comprises the full length HIV gag gene. This plasmid is also referred to as “pV1JnsBJVgag-bGHpA”, pV1Jns-hCMV-FL-gag-bGHpA” and “pV1JnsCMV(no intron)+FLgag+bGHpA”.

“pV1JnsCMV(no intron)-FLgag-SPA” is a plasmid of the same composition as pV1JnsCMV(no intron)-FLgag-bGHpA except that the SPA termination sequence replaces that of bGHpA. This plasmid is also referred to as “pV1Jns-HIVgag-SPA” and pV1Jns-hCMV-FLgag-SPA”.

“pdelE1sp1A” is a universal shuttle vector with no expression cassette (i.e., no promoter or polyA). The vector comprises wildtype adenovirus serotype 5 (Ad5) sequences from bp 1 to bp 341 and bp 3524 to bp 5798, and has a multiple cloning site between the Ad5 sequences ending 341 bp and beginning 3524 bp. This plasmid is also referred to as the original Ad 5 shuttle vector.

“MRKpdelE1sp1A” or “MRKpdelE1 (Pac/pIX/pack450)” or “MkKpdelE1(Pac/pIX/pack450)Cla1” is a universal shuttle vector with no expression cassette (i.e. no promoter or polyA) comprising wildtype adenovirus serotype 5 (Ad5) sequences from bp 1 to bp 450 and bp 3511 to bp 5798. The vector has a multiple cloning site between the Ad5 sequence ending 450 bp and beginning 3511 bp. This shuttle vector may be used to insert the CMV promoter and the bGHpA fragments in both the straight (“str”. or E1 parallel) orientation or in the opposite (opp. or E1 antiparallel) orientation.

“MRKpdelE1(Pac/pIX/pack450)+CMVmin+BGHpA(str.)” is still another shuttle vector which is the modified vector that contains the CMV promoter (no intron A) and the bGHpA fragments. The expression unit containing the hCMV promoter (no intron A) and the bovine growth hormone polyadenylation signal has been inserted into the shuttle vector such that insertion of the gene of choice at a unique BglII site will ensure the direction of transcription of the transgene will be Ad5 E1 parallel when inserted into the MRKpAd5(E1/E3+)Cla1 pre-plasmid.

“MRKpdelE1-CMV(no intron)-FLgag-bGHpA” is a shuttle comprising Ad5 sequences from base pairs 1-450 and 3511-5798, with an expression cassette containing human CMV without intron A, the full-length human codon-optimized HIV gag gene and bovine growth hormone polyadenylation signal. This plasmid is also referred to as “MRKpdelE1 shuttle +hCMV-FL-gag-BGHpA”

“MRKpAdHVE3+CMV(no intron)-FLgag-bGHpA” is an adenoviral vector comprising all Ad5 sequences except those nucleotides encompassing the E1 region (from 451-3510), a human CMV promoter without intron A, a full-length human codon-optimized HIV gag gene, and a bovine growth hormone polyadenylation signal. This vector is also referred to as “MRKpAdHVE3+hCMV-FL-gag-BGHpA”, “MRKpAdSHIV-1gag”, “MRKpAd5gag”, “pMRKAd5gag” or “pAd5gag2”.

### BRIEF DESCRIPTION OF THE FIGURES

FIG. 1 shows the HIV-1 gag adenovector “Ad5 HIV-1 gag”. This vector is disclosed in PCT International Application No. PCT/US00/18332 (WO 01/02607) filed Jul. 3, 2000, claiming priority to U.S. Provisional Application Ser. No. 60/142,631, filed Jul. 6, 1999, and U.S. Application Ser. No. 60/148,981, filed Aug. 13, 1999, all three applications which are hereby incorporated by reference.

FIG. 2 shows the nucleic acid sequence (SEQ ID NO: 1) of the optimized human HIV-1 gag open reading frame.

FIG. 3 shows diagrammatically the transgene construct disclosed in PCT International Application No. PCT/US01/28861, filed Sep. 14, 2001 in comparison with the original gag transgene. PCT International Application No. PCT/US01/28861 claims priority to U.S. Provisional Application Ser. Nos. 60/233,180, 60/279,056, and 60/317,814, filed Sep. 15, 2000, Mar. 27, 2001, and Sep. 7, 2001, respectively; the above applications all of which are hereby incorporated by reference.

FIG. 4 shows the modifications made to the adenovector backbone of Ad5HIV-1 gag in the generation of the vector disclosed in PCT International Application No. PCT/US01/28861 which is utilized in certain examples of the instant application.

FIG. 5 shows the levels of Gag-specific T cells in rhesus macaques immunized with (a) two priming doses of 10e9 vp of MRKAd5HIV-1 gag and a single booster shot with 10e9 vp MKAd5 HIV-1 gag (“10e9 vp MRKAd5-10e9 vp MRKAd5”); (b) two priming doses of 10e9 pfu MRKAd6 HIV-1 gag and a single booster with 10e9 pfu MRKAd6 HIV-1 gag (“10e9 pfu MRKAd6-10e9 pfu MRKAd6”); or (c) two priming doses of 10e9 vp of MRKAd5 HIV-1 gag followed by a single booster shot with 10e9 pfu MRKAd6 HIV-1 gag (“10e9 vp MRKAd5-10e9 pfu MRKAd6”). The levels expressed as number of spot-forming cells (SFC) per million PBMC are the mock-corrected values for each animal prior to the start of the immunization regimen (“Pre”); 4 weeks after the first priming dose (“Post Dose 1”); 4 weeks after the second priming dose (“Post Dose 2”); just prior to the boost (“Pre-Boost”); 4 weeks after the boost (“4 wks Post-Boost”); and 8 weeks after the boost (“8 wks Post-Boost”).

FIG. 6 shows the Gag-specific T cell responses induced by two priming doses of 10e7 vp dose of MRKAd5HIV-1 gag (week 0; week 4) followed by administration of 10e7 vp MRKAd6 HIV-1 gag at week 27. The levels provided are the mock-corrected levels for each animal prior to the start of the immunization regimen (“Pre”); 4 weeks after the first priming dose (“Post Dose 1”); 4 weeks after the second priming dose (“Post Dose 2”); just prior to the boost (“Pre-Boost”); 4 weeks after the boost (“4 wks Post-Boost”); and 8 weeks after the boost (“8 wks Post-Boost”). One will note a significant increase compared to the levels just prior to the boost. MRKAd6 HIV-1 gag elicited a large amplification of the priming response. The post-boost increases shown are largely attributed to the expansion of memory T cells instead of priming of new lymphocytes.

FIG. 7 shows the homologous recombination protocol utilized to recover pAdE1-E3 disclosed herein.

FIG. 8 shows a restriction map of the pMRKAd5HIV-1gag vector.

FIGS. 9A-1 to 9A-45 show the nucleotide sequence of the pMRKAd5HIV-1gag vector (SEQ ID NO:2 [coding] and SEQ ID NO:3 [non-coding]).

FIG. 10 shows the levels of Gag-specific antibodies in rhesus macaques immunized with (a) two priming doses of 10e9 vp of MRKAd5 HIV-1 gag and a single booster shot with 10e9 vp MRKAd5 HIV-1 gag (“10e9 vp MRKAd5-10e9 vp MRKAd5”), (b) two priming doses of 10e9 pfu MRKAd6 HIV-1 gag and a single booster with 10e9 pfu MKAd6 HIV-1 gag (“10e9 pfu MRKAd6-10e9 pfu MRKAd6”), or (c) two priming doses of 10e9 vp of MRKAd5 HIV-1 gag followed by a single booster shot with 10e9 pfu MRKAd6 HIV-1 gag (“10e9 vp MRKAd5-10e9 pfu MRKAd6”). Shown are the geometric mean titers for each cohort at the start of the immunization regimen (“Pre”), 4 weeks after the first priming dose (“Wk 4”), 4 weeks after the second priming dose (“Wk 8”), just prior to the boost (“Pre-Boost”), and 8 weeks after the boost (“Post-Boost”).

FIGS. 11A-1 to 11A-14 show the nucleic acid sequence for the Ad6 genome (SEQ ID NO:5).

FIG. 12 shows the basic genomic organization of Ad6. The linear (35759 bp) double-stranded DNA genome is indicated by two parallel lines and is divided into 100 map units. Transcription units are shown relative to their position and orientation in the genome. Early genes (E1A, E1B, E2A/B, E3 and E4) are indicated by gray bars. Late genes (L1 to L5), indicated by black bars, are produced by alternative splicing of a transcript produced from the major late promoter (ILP) and all contain the tripartite leader (1, 2, 3) at their 5′ ends.

FIG. 13 shows the homologous recombination protocol utilized to recover pMRKAd6E1-.

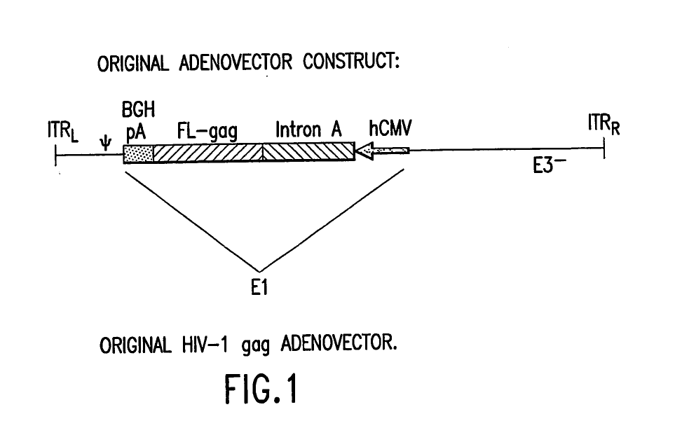

### DETAILED DESCRIPTION OF THE INVENTION

An enhanced means for generating an immune response against human immunodeficiency virus (“HIV”) is described. The disclosed methods employ a combination of recombinant adenovirus gene delivery vehicles of alternative and distinct serotypes in the administration of exogenous genetic material encoding an HIV antigen (or antigens) of interest. In accordance with the methods of the instant invention, a priming dose of the HIV antigen(s) is first delivered with a recombinant adenoviral vector of a first serotype. This dose effectively primes the immune response so that, upon subsequent identification of the antigen in the circulating immune system, the immune response is capable of immediately recognizing and responding to the antigen within the host. The priming dose(s) is then followed up with a boosting dose of a second and different adenovirus serotype comprising exogenous genetic material encoding the antigen. In one aspect of the instant invention, a mammalian host is first administered a priming dose(s) comprising a recombinant adenoviral vector of serotype 5 or 6 and then administered a subsequent boosting dose(s) comprising a recombinant adenoviral vector of a different serotype (i.e., a serotype other than that used in the priming administration; examples, but not limitations of which include Ad35. Very specific embodiments encompassed herein are wherein (1) an Ad5-primed response is boosted with a recombinant Ad6 vehicle comprising an HIV antigen; (2) an Ad6-primed response is boosted with a recombinant Ad5 vehicle comprising an HIV antigen; (3) an Ad5/Ad6-primed response is boosted with a recombinant, Ad35-based vehicle; and (4) an Ad35-primed response is boosted with a recombinant, an Ad5/Ad6-based vehicle. As relates to HIV antigens, administration in accordance with the methods of the instant invention results in a significant non-additive synergistic effect which notably increases the immune response seen in inoculated mammalian hosts. The effects are particularly evident in the cellular immune responses generated following inoculation. The disclosed immunization regime, thus, offers a prophylactic advantage to previously uninfected individuals and can offer a therapeutic effect to reduce viral load levels in those already infected with the virus, thus prolonging the asymptomatic phase of HIV-1 infection.

Accordingly, the instant invention relates to a method for inducing an enhanced immunological response against an HIV-1 antigen in a mammalian host comprising the steps of (a) inoculating the mammalian host with a recombinant adenoviral vector of a first serotype at least partially deleted in E1 and devoid of E1 activity comprising a gene encoding an HIV-1 antigen or immunologically relevant modification thereof; and thereafter (b) inoculating the mammalian host with a boosting immunization comprising a recombinant adenovirus vector of a second and distinct serotype at least partially deleted in E1 and devoid of E1 activity comprising a gene encoding an HIV-1 antigen or immunologically relevant modification thereof. Preferred embodiments of the instant invention employ adenoviral vectors which are replication-defective by reason of having a deletion in the E1 region which renders the vector devoid (or essentially devoid) of E1 activity. Adenovirus serotype 5 has been found to be a very effective adenovirus vehicle for purposes of effectuating sufficient expression of exogenous genetic material encoding HIV-specific antigens in order to provide for sufficient priming of the mammalian host immune response. It has further been found and disclosed herein that recombinant adenovirus serotype 6 is capable of very effectively boosting the adenovirus serotype 5-primed response. In an alternative scenario, recombinant adenovirus serotype 5 can be used to boost an adenovirus serotype 6-primed response. These findings have also been demonstrated with adenovirus vehicles of different subgroups, for instance, Ad5/6-prime (subgroup C)/Ad35-boost (subgroup B).

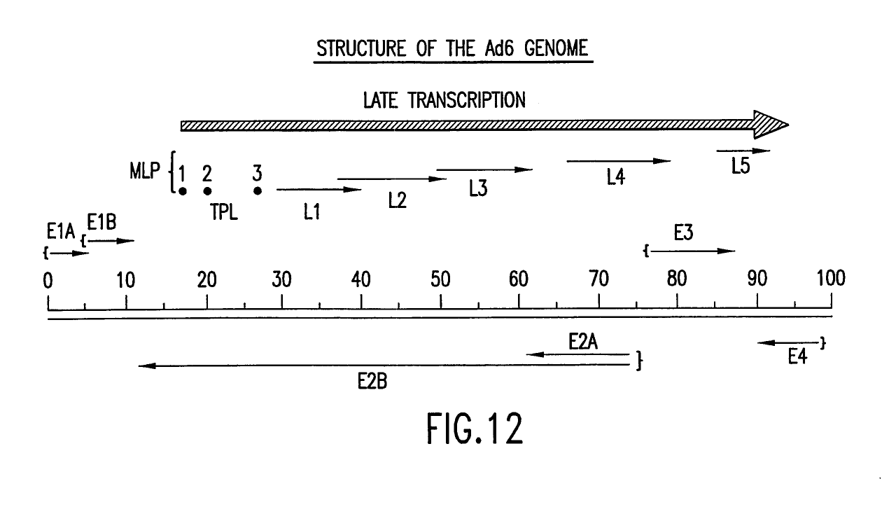

The wildtype adenovirus serotype 5 sequence is known and described in the art; see, Chroboczek et al., 1992 J. Virology 186:280, which is hereby incorporated by reference. Accordingly, a particular embodiment of the instant invention is an immunization scheme employing an adenovirus vehicle based on the wildtype adenovirus serotype 5 sequence in the priming or boosting administration; a virus of which is on deposit with the American Type Culture Collection (“ATCC”) under ATCC Deposit No. VR-5. One of skill in the art can, however, readily identify alternative and distinct adenovirus serotypes (e.g., serotypes 2, 4, 6, 12, 16, 17, 24, 31, 33. and 42) and incorporate same in the disclosed heterologous prime-boost immunization schemes. The sequence of adenovirus serotype 6 (ATCC Deposit No. VR-6) is extremely homologous (approximately 98%) at the nucleic acid level to the sequence of adenovirus serotype 5, with relatively few base pair differences in the approximate 36 kb sequences. The genomic organization of Ad6 is also very similar; see FIG. 12. Chimeric Ad5/Ad6 constructs which retain the serotype-determining epitopes of either Ad5 or Ad6 are also suitable for use in the instant invention; provided that the serotype determining epitopes are distinct from the adenovirus vehicle used in combination therewith (i.e., that the determinants are distinct from the vehicle used in the priming dose if the chimera is utilized in the boosting dose, and vice versa). It is important to the overall functioning of the disclosed methods that the serotypes of the priming and boosting vectors be distinct.

Recombinant adenoviral vectors comprising deletions additional to that contained within the region of E1 are also contemplated for use within the methods of the instant invention. For example, vectors comprising deletions in both E1 and E3 are contemplated for use within the methods of the instant invention. Such a vector can accommodate a larger amount of foreign DNA (or exogenous genetic material).

Adenoviral vectors of use in the methods of the instant invention can be constructed using known techniques, such as those reviewed in Hitt et al., 1997 “Human Adenovirus Vectors for Gene Transfer into Mammalian Cells” Advances in Pharmacology 40:137-206, which is hereby incorporated by reference. Often, a plasmid or shuttle vector is generated which comprises sequence from the specific adenovirus of interest. This process is described in Hitt et al., supra.

Adenoviral pre-plasmids (e.g., pMRKAd5gag and pMRKAd6gag) can be generated by homologous recombination using adenovirus backbones (e.g., MRKAd5HVE3 and pMRKAd6E1-, an Ad6 genome plasmid) and the appropriate shuttle vector. The resultant plasmids in linear form, are capable of replication after entering the PER.C6® cells or other complementing cell line, and virus is produced. The infected cells and media are then harvested after viral replication is complete.

Viral vectors can be propagated in various E1 complementing cell lines, including the known cell lines 293 and PER.C6®. Both these cell lines express the adenoviral E1 gene product. PER.C6® is described in WO 97/00326 (published Jan. 3, 1997) and issued U.S. Pat. No. 6,033,908, both of which are hereby incorporated by reference. It is a primary human retinoblast cell line transduced with an E1 gene segment that complements the production of replication deficient (FG) adenovirus, but is designed to prevent generation of replication competent adenovirus by homologous recombination. Cells of particular interest have been stably transformed with a transgene that encodes the AD5E1A and E1B gene, like PER.C6®, from 459 bp to 3510 bp inclusive. 293 cells are described in Graham et al., 1977 J. Gen. Virol 36:59-72, which is hereby incorporated by reference. As stated above, due consideration must be given to the adenoviral sequences present in the complementing cell line used. It is preferred that the sequences not overlap with that present in the vector if the possibility of recombination is to be minimized.

The recombinant adenoviral vectors of use in the instant invention comprise a gene encoding any antigen, but particularly, an HIV-1 antigen or an immunologically relevant modification thereof. HIV antigens of interest include, but are not limited to, the major structural proteins of HIV such as Gag, Pol, and Env, immunologically relevant modifications, and immunogenic portions thereof. The invention, thus, encompasses the various forms of codon-optimized HIV-1 gag (including but by no means limited to p55 versions of codon-optimized full length (“FL”) Gag and tPA-Gag fusion proteins), HIV-1 pol, HIV-1 nef, HIV-1 env, and selected modifications of immunological relevance.

Exogenous genetic material encoding a protein of interest may exist in the form of an expression cassette. A gene expression cassette preferably comprises (a) a nucleic acid encoding a protein of interest; (b) a heterologous (non-native) or modified native promoter operatively linked to the nucleic acid encoding the protein; and (c) a transcription termination sequence.

The transcriptional promoter is preferably recognized by an eukaryotic RNA polymerase. In a preferred embodiment, the promoter is a “strong” or “efficient” promoter. An example of a strong promoter is the immediate early human cytomegalovirus promoter (Chapman et al, 1991 Nucl. Acids Res. 19:3979-3986, which is incorporated by reference); in certain embodiments without intronic sequences. Specific embodiments of the instant invention employ human CMV promoters without intronic sequences, like intron A. Applicants have found that intron A, a portion of the human cytomegalovirus promoter (hCMV), constitutes a region of instability for adenoviral vectors. CMV without intron A has been found to effectuate comparable expression capabilities in vitro when driving HIV gag expression and, furthermore, behaved equivalently to intron A-containing constructs in Balb/c mice in vivo with respect to their antibody and T-cell responses at both dosages of plasmid DNA tested (20 μg and 200 μg). Those skilled in the art will appreciate that any of a number of other known promoters, such as the strong immunoglobulin, or other eukaryotic gene promoters may also be used, including the EF1 alpha promoter, the murine CMV promoter, Rous sarcoma virus (RSV) promoter, SV40 early/late promoters and the beta-actin promoter. In certain embodiments, the promoter may also comprise a regulatable sequence such as the Tet operator sequence. This would be extremely useful, for example, in cases where the gene products are effecting a result other than that desired and repression is sought. Preferred transcription termination sequences present within the gene expression cassette are the bovine growth hormone terminator/polyadenylation signal (bGHpA) and the short synthetic polyA signal (SPA) of 50 nucleotides in length, defined as follows: AATAAAAGATCTTTATTTTCATTAGATCTGTGTGTTGGT-TTTTTGTGTG (SEQ ID NO:4). The combination of the CMV promoter (devoid of the intron A region) with the BGH terminator constitutes a specific embodiment of the present invention, although other promoter/terminator combinations can be used. Certain embodiments may incorporate a leader or signal peptide into the transgene. A preferred leader is that from the tissue-specific plasminogen activator protein, tPA.

In accordance with the methods of the instant invention, the expression of exogenous HIV genetic material should elicit potent and broad cellular immune responses against HIV that will either lessen the likelihood of persistent virus infection and/or lead to the establishment of a clinically significant lowered virus load subject to HIV infection or in combination with HAART therapy, mitigate the effects of previously established HIV infection (antiviral immunotherapy (ARI)). While any HIV antigen (e.g., gag, pol, nef, gp160, gp41, gp120, tat, rev, etc.) may be incorporated into the recombinant adenoviral vectors of use in the instant invention, preferred embodiments include the codon optimized p55 gag antigen, pol and nef. The adenoviral vehicles of the instant invention can utilize heterologous nucleic acid which may or may not be codon-optimized. In specific embodiments of the instant invention, the individual can be primed with an adenoviral vector comprising codon-optimized heterologous nucleic acid, and boosted with an adenovirus of an alternative serotype comprising non-codon-optimized nucleic acid. Administration of multiple antigens possesses the possibility for exploiting various different combinations of codon-optimized and non-codon-optimized sequences.

Sequences based on different Clades of HIV-1 are suitable for use in the instant invention, most preferred of which are Clade B and Clade C. Particularly preferred embodiments are those sequences (especially, codon-optimized sequences) based on consensus Clade B sequences. Preferred versions of the viral vaccines will encode modified versions of pol or nef. Preferred embodiments of the viral vaccines carrying HIV envelope genes and modifications thereof comprise the HIV codon-optimized env sequences of PCT International Applications PCT/US97/02294 and PCT/US97/10517, published Aug. 28, 1997 (WO 97/31115) and Dec. 24, 1997, respectively; both documents of which are hereby incorporated by reference.

Sequences for many genes of many HIV strains are publicly available in GENBANK and primary, field isolates of HIV are available from the National Institute of Allergy and Infectious Diseases (NIAID) which has contracted with Quality Biological (Gaithersburg, Md.) to make these strains available. Strains are also available from the World Health Organization (WHO), Geneva Switzerland. It is preferred that the gag gene be from an HIV-1 strain (CAM-1; Myers et al, eds. “Human Retroviruses and AIDS: 1995, IIA3-IIA19, which is hereby incorporated by reference). This gene closely resembles the consensus amino acid sequence for the clade B (North American/European) sequence. Therefore, it is within the purview of the skilled artisan to choose an appropriate nucleotide sequence which encodes a specific HIV gag antigen, or immunologically relevant portion thereof. A clade B or clade C based p55 gag antigen will potentially be useful on a global scale. A transgene of choice for insertion into the vectors utilized within the disclosed methods is a codon-optimized version of p55 gag.

In addition to a single HIV antigen of interest being delivered by the recombinant adenoviral vectors, two or more antigens can be delivered either via separate vehicles or delivered via the same vehicle. For instance, a priming dose in accordance with the instant invention can comprise a recombinant adenoviral vector of a first serotype comprising genes encoding both nef and pol or, alternatively, two or more alternative HIV-1 antigens. The boosting dose could then comprise a recombinant adenoviral vector of a second and different serotype comprising the genes encoding both nef and pol (or whichever two or more HIV-1 antigens were used in the priming dose). In an alternative scenario, the priming dose can comprise a mixture of separate adenoviral vehicles each comprising a gene encoding for a different HIV-1 antigen. In such a case, the boosting dose could also comprise a mixture of vectors each comprising a gene encoding for a separate HIV-1 antigen, provided that the boosting dose(s) administers recombinant viral vectors comprising genetic material encoding for the same or a similar set of antigens that were delivered in the priming dose(s). These divalent (e.g., gag and nef, gag and pol, or pol and nef components, for instance) or trivalent (e.g., gag, pol and nef components, for instance) vaccines can further be administered by a combination of the techniques described above. Therefore, a preferred aspect of the present invention are the various vaccine formulations that can be administered by the methods of the instant invention. It is also within the scope of the present invention to embark on combined modality regimes which include multiple but distinct components from a specific antigen.

The disclosed immunization regimes employing fusion constructs composed of two or more antigens are also encompassed herein. For example, multiple HIV-L viral antigens may be ligated into a proper shuttle plasmid for generation of a pre-viral plasmid comprising multiple open reading frames. For example a trivalent vector may comprise a gag-pol-nef fusion, or possible a “2+1” divalent vaccine comprising, for instance, a gag-pol fusion (e.g., a codon optimized p55 gag and inactivated optimized pol) with each open reading frame being operatively linked to a distinct promoter and transcription termination sequence. Alternatively, the two open reading frames in the same construct may be operatively linked to a single promoter, with the open reading frames operatively linked by an internal ribosome entry sequence (IRES), as disclosed in International Publication No. WO 95/24485, which is hereby incorporated by reference. In the absence of the use of IRES-based technology, it is preferred that a distinct promoter be used to support each respective open reading frame, so as to best preserve vector stability. As examples, and certainly not as limitations, potential multiple transgene vaccines may include a three transgene vector such as that wherein a gagpol fusion and nef gene were included in the same vector with different promoters and termination sequences being used for the gagpol fusion and nef gene. Further, potential “2+1” divalent vaccines of the present invention might be wherein a construct containing gag and nef in the same construct with separate promoters and termination sequences is administered in combination with a construct comprising a pol gene with promoter and termination sequence. Fusion constructs other than the gag-pol fusion described above are also suitable for use in various divalent vaccine strategies and can be composed of any two HIV antigens fused to one another (e.g., nef-pol and gag-nef). These compositions are, as above, preferably delivered along with a viral composition comprising an additional HIV antigen in order to diversify the immune response generated upon inoculation. Therefore, a multivalent vaccine delivered in a single, or possibly second, viral vector is certainly contemplated as part of the present invention. It is important to note, however, that in terms of deciding on an insert for the disclosed viral vectors, due consideration must be given to the effective packaging limitations of the viral vehicle.

Adenovirus, for instance, has been shown to exhibit an upper cloning capacity limit of approximately 105% of the wildtype Ad5 sequence.

Regardless of the gene chosen for expression, it is preferred that the sequence be “optirnized” for expression in a mammalian (e.g., human) cellular environment, particularly in the adenoviral constructs. A “triplet” codon of four possible nucleotide bases can exist in 64 variant forms. That these forms provide the message for only 20 different amino acids (as well as transcription initiation and termination) means that some amino acids can be coded for by more than one codon. Indeed, some amino acids have as many as six “redundant”, alternative codons while some others have a single, required codon. For reasons not completely understood, alternative codons are not at all uniformly present in the endogenous DNA of differing types of cells and there appears to exist variable natural hierarchy or “preference” for certain codons in certain types of cells. As one example, the amino acid leucine is specified by any of six DNA codons including CTA, CTC, CTG, CTT, TTA, and TTG (which correspond, respectively, to the mRNA codons, CUA, CUC, CUG, CUU, UUA and UUG). Exhaustive analysis of genome codon frequencies for microorganisms has revealed endogenous DNA of E. coli most commonly contains the CTG leucine-specifying codon, while the DNA of yeast and slime molds most commonly includes a TTA leucine-specifying codon. In view of this hierarchy, it is generally held that the likelihood of obtaining high levels of expression of a leucine-rich polypeptide by an E. coli host will depend to some extent on the frequency of codon use. For example, a gene rich in TTA codons will in all probability be poorly expressed in E. coli, whereas a CTG rich gene will probably highly express the polypeptide. Similarly, when yeast cells are the projected transformation host cells for expression of a leucine-rich polypeptide, a preferred codon for use in an inserted DNA would be TTA.

The implications of codon preference phenomena on recombinant DNA techniques are manifest, and the phenomenon may serve to explain many prior failures to achieve high expression levels of exogenous genes in successfully transformed host organisms—a less “preferred” codon may be repeatedly present in the inserted gene and the host cell machinery for expression may not operate as efficiently. This phenomenon suggests that synthetic genes which have been designed to include a projected host cell's preferred codons provide a preferred form of foreign genetic material for practice of recombinant DNA techniques. Thus, one aspect of this invention is a vaccine administration protocol wherein the recombinant adenoviral vectors (prime and boost vectors) specifically include a gene which is codon optimized for expression in a human cellular environment. As noted herein, a preferred gene for use in the instant invention is a codon-optimized HIV gene and, particularly, HIV gag, pol, env, or nef although, as stated above, the adenoviral vehicles of the instant invention can utilize heterologous nucleic acid which may or may not be codon-optimized. In specific embodiments of the instant invention, the individual can be primed with an adenoviral vector comprising codon-optimized heterologous nucleic acid, and boosted with an adenovirus of an alternative serotype comprising non-codon-optimized nucleic acid. Administration of multiple antigens possesses the possibility for exploiting various different combinations of codon-optimized and non-codon-optimized sequences.

A vaccine composition comprising the recombinant viral vectors either in the priming or boosting dose in accordance with the instant invention may contain physiologically acceptable components, such as buffer, normal saline or phosphate buffered saline, sucrose, other salts and polysorbate. One preferred formulation has: 2.5-10 mM TRIS buffer, preferably about 5 mM TRIS buffer; 25-100 mM NaCl, preferably about 75 mM NaCl; 2.5-10% sucrose, preferably about 5% sucrose; 0.01-2 mM MgCl₂; and 0.001%-0.01% polysorbate 80 (plant derived). The pH should range from about 7.0-9.0, preferably about 8.0. One skilled in the art will appreciate that other conventional vaccine excipients may also be used it make the formulation. The preferred formulation contains 5 mM TRIS, 75 mM NaCl, 5% sucrose, 1 mM MgCl₂, 0.005% polysorbate 80 at pH 8.0. This has a pH and divalent cation composition which is near the optimum for Ad5 and Ad6 stability and minimizes the potential for adsorption of virus to a glass surface. It does not cause tissue irritation upon intramuscular injection. It is preferably frozen until use.

The amount of viral particles in the vaccine composition to be introduced into a vaccine recipient will depend on the strength of the transcriptional and translational promoters used and on the immunogenicity of the expressed gene product. In general, an immunologically or prophylactically effective dose of 1×10⁷ to 1×10¹² particles and preferably about 1×10¹¹ to 1×10¹¹ particles is administered directly into muscle tissue. Subcutaneous injection, intradermal introduction, impression through the skin, and other modes of administration such as intraperitoneal, intravenous, or inhalation delivery are also contemplated. Parenteral administration, such as intravenous, intramuscular, subcutaneous or other means of administration of interleukin-12 protein, concurrently with or subsequent to parenteral introduction of the vaccine compositions of this invention is also advantageous.

The administration schemes of the instant invention are based on the priming of the immune response with an adenoviral vehicle of a first serotype comprising a gene encoding an HIV antigen (or antigens) and, following a predetermined length of time, boosting the adenovirus-primed response with an adenoviral vehicle of a second and alternative serotype comprising the gene encoding the HIV antigen(s). Multiple primings, typically, 14, are usually employed, although more may be used. The length of time between prime and boost may typically vary from about four months to a year, but other time frames may be used. The booster dose may be repeated at selected time intervals.

A large body of human and animal data supports the importance of cellular immune responses, especially CTL in controlling (or eliminating) HIV infection. In humans, very high levels of CTL develop following primary infection and correlate with the control of viremia. Several small groups of individuals have been described who are repeatedly exposed to HIV but remain uninfected; CTL has been noted in several of these cohorts. In the SIV model of HIV infection, CTL similarly develops following primary infection, and it has been demonstrated that addition of anti-CD8 monoclonal antibody abrogated this control of infection and leads to disease progression.

The following non-limiting Examples are presented to better illustrate the invention.

### EXAMPLE 1

### HIV-1 Gag Gene

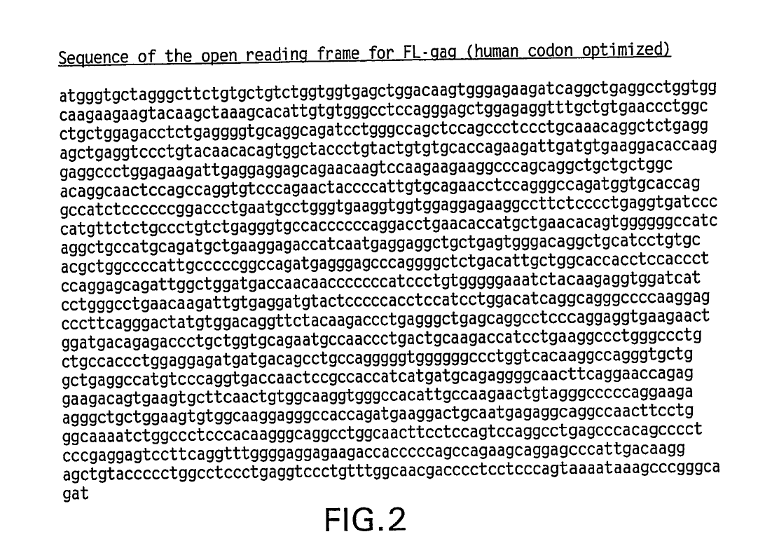

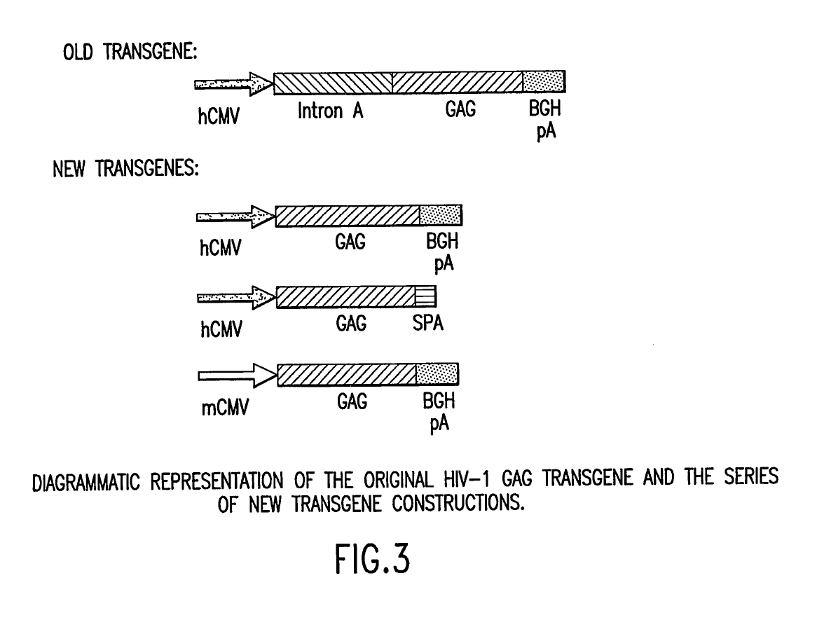

A synthetic gene for HIV gag from HIV-1 strain CAM-1 was constructed using codons frequently used in humans; see Korber et al., 1998 Human Retroviruses and AIDS, Los Alamos Nat'l Lab., Los Alamos, N. Mex.; and Lathe, R., 1985 J. Mol. Biol. 183:1-12. FIG. 2 illustrates the nucleotide sequence of the exemplified optimized codon version of full-length p55 gag. The gag gene of HIV-1 strain CAM-1 was selected as it closely resembles the consensus amino acid sequence for the lade B (North American/European) sequence (Los Alamos HIV database). Advantage of this “codon-optimized” HIV gag gene as a vaccine component has been demonstrated in immunogenicity studies in mice. The “codon-optimized” HIV gag gene was shown to be over 50-fold more potent to induce cellular immunity than the wild type HIV gag gene when delivered as a DNA vaccine.

A KOZAK sequence (GCCACC) was introduced proceeding the initiating ATG of the gag gene for optimal expression. The HIV gag fragment with KOZAK sequence was amplified through PCR from V1Jns-HIV gag vector. PVIJnsHIVgag is a plasmid comprising the CMV immediate-early (E) promoter and intron A, a full-length codon-optimized HIV gag gene, a bovine growth hormone-derived polyadenylation and transcriptional termination sequence, and a minimal pUC backbone; see Montgomery et al., 1993 DNA Cell Biol. 12:777-783, for a description of the plasmid backbone.

### EXAMPLE 2

### Generation of Adenoviral Serotype 5 Vector Constructs

A. Removal of the Intron A Portion of the hCMV Promoter

GMP grade pVIJnsHIVgag was used as the starting material to amplify the hCMV promoter. The amplification was performed with primers suitably positioned to flank the hCMV promoter. A 5′ primer was placed upstream of the Msc1 site of the hCMV promoter and a 3′ primer (designed to contain the BglII recognition sequence) was placed 3′ of the hCMV promoter. The resulting PCR product (using high fidelity Taq polymerase) which encompassed the entire hCMV promoter (minus intron A) was cloned into TOPO PCR blunt vector and then removed by double digestion with Msc1 and BglII. This fragment was then cloned back into the original GMP grade pV1JnsHIVgag plasmid from which the original promoter, intron A, and the gag gene were removed following Msc1 and BglII digestion. This ligation reaction resulted in the construction of a hCMV promoter (minus intron A)+bGHpA expression cassette within the original pV1JnsHIVgag vector backbone. This vector is designated pVIJnsCMV(no intron).

The FLgag gene was excised from pV1JnsHIVgag using BglII digestion and the 1,526 bp gene was gel purified and cloned into pV1JnsCMV(no intron) at the BglII site. Colonies were screened using Sma1 restriction enzymes to identify clones that carried the FLgag gene in the correct orientation. This plasmid, designated pV1JnsCMV(no intron)-FLgag-bGHpA, was fully sequenced to confirm sequence integrity.

B. Construction of the Modified Shuttle Vector—“MkKpdelE1 Shuttle”

The modifications to the original Ad5 shuttle vector (pdelE1sp1A; a vector comprising Ad5 sequences from base pairs 1-341 and 3524-5798, with a multiple cloning region between nucleotides 341 and 3524 of Ad5, included the following three manipulations carried out in sequential cloning steps as follows:

(1) The left ITR region was extended to include the Pac1 site at the junction between the vector backbone and the adenovirus left ITR sequences. This allow for easier manipulations using the bacterial homologous recombination system.

(2) The packaging region was extended to include sequences of the wild-type (WT) adenovirus from 342 bp to 450 bp inclusive.

(3) The area downstream of pix was extended 13 nucleotides (i.e., nucleotides 3511-3523 inclusive).

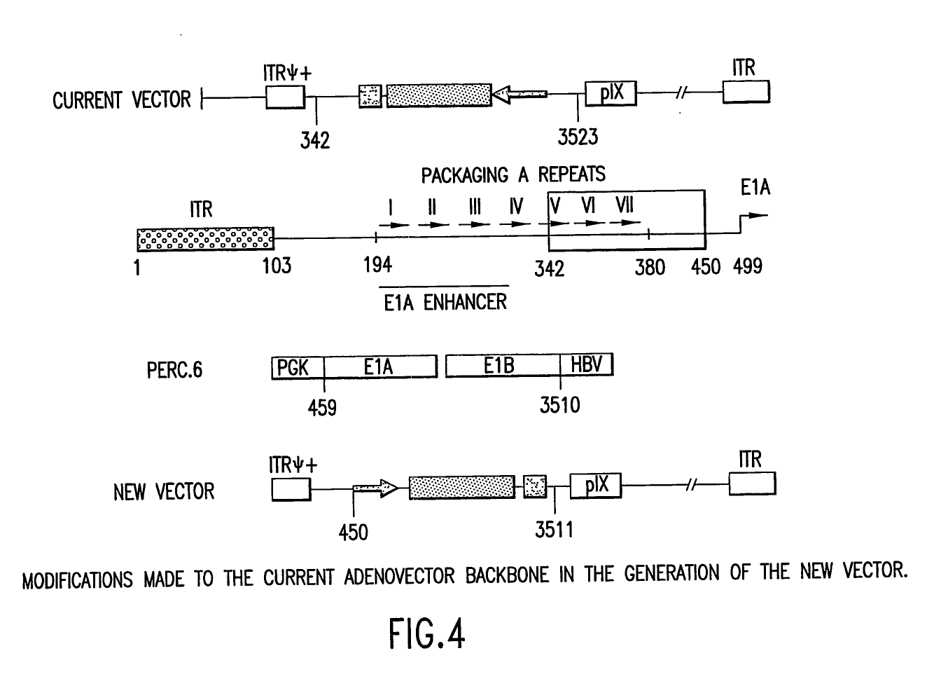

These modifications (FIG. 4) effectively reduced the size of the E1 deletion without overlapping with any part of the E1A/E1B gene present in the transformed PER.C6® cell line. All manipulations were performed by modifying the Ad shuttle vector pdelE1sp1A.

Once the modifications were made to the shuttle vector, the changes were incorporated into the original Ad5 adenovector backbone pAdHVE3 by bacterial homologous recombination using E. coli BJ5183 chemically competent cells.

C. Construction of Modified Adenovector Backbone

An original adenovector pADHVE3 (comprising all Ad5 sequences except those nucleotides encompassing the E1 region) was reconstructed so that it would contain the modifications to the E1 region. This was accomplished by digesting the newly modified shuttle vector (MRKpdelE1 shuttle) with Pac1 and BstZ1101 and isolating the 2,734 bp fragment which corresponds to the adenovirus sequence. This fragment was co-transformed with DNA from Cla1 linearized pAdHVE3 (E3+adenovector) into E. coli BJ5183 competent cells. At least two colonies from the transformation were selected and grown in Terrific™ broth for 6-8 hours until turbidity was reached. DNA was extracted from each cell pellet and then transformed into E. coli XL1 competent cells. One colony from the transformation was selected and grown for plasmid DNA purification. The plasmid was analyzed by restriction digestions to identify correct clones. The modified adenovector was designated MRKpAdHVE3 (E3+ plasmid). Virus from the new adenovector (MRKHVE3) as well as the old version were generated in the PER.C6® cell lines. In addition, the multiple cloning site of the original shuttle vector contained ClaI, BamHI, Xho I, EcoRV, HindIII, Sal I, and Bgl II sites. This MCS was replaced with a new MCS containing Not I, Cla I, EcoRV and Asc I sites. This new MCS has been transferred to the MRKpAdHVE3 pre-plasmid along with the modification made to the packaging region and pIX gene.

D. Construction of the New Shuttle Vector Containing Modified gag Transgene—“MRKpdelE1-CMV(No Intron)-FLgag-bGHpA”

The modified plasmid pV1JnsCMV(no intron)-FLgag-bGHpA was digested with Msc1 overnight and then digested with Sfi1 for 2 hours at 50° C. The DNA was then treated with Mungbean nuclease for 30 minutes at 30° C. The DNA mixture was desalted using the Qiaex II kit and then Klenow treated for 30 minutes at 37° C. to fully blunt the ends of the transgene fragment. The 2,559 bp transgene fragment was then gel purified. The modified shuttle vector (MRKpdelE1 shuttle) was linearized by digestion with EcoRV, treated with calf intestinal phosphatase and the resulting 6,479 bp fragment was then gel purified. The two purified fragments were then ligated together and several dozen clones were screened to check for insertion of the transgene within the shuttle vector. Diagnostic restriction digestion was performed to identify those clones carrying the transgene in the E1 parallel orientation.

E. Construction of the MRK FG Adenovector

The shuttle vector containing the HIV-1 gag transgene in the E1 parallel orientation, MRKpdelE1-CMV(no intron)-FLgag-bGHpA, was digested with Pac1. The reaction mixture was digested with BsfZ171. The 5,291 bp fragment was purified by gel extraction. The MRKpAdHVE3 plasmid was digested with ClaI overnight at 37° C. and gel purified. About 100 ng of the 5,290 bp shuttle +transgene fragment and ˜100 ng of linearized MRKpAdHVE3 DNA were co-transformed into E. coli BJ5183 chemically competent cells. Several clones were selected and grown in 2 ml Terrific™ broth for 6-8 hours, until turbidity was reached. The total DNA from the cell pellet was purified using Qiagen alkaline lysis and phenol chloroform method. The DNA was precipitated with isopropanol and resuspended in 20 μL dH₂O. A 2 μl aliquot of this DNA was transformed into E. coli XL-1 competent cells. A single colony from the transformation was selected and grown overnight in 3 ml LB +100 μg/ml ampicillin. The DNA was isolated using Qiagen columns. A positive clone was identified by digestion with the restriction enzyme BstEII which cleaves within the gag gene as well as the plasmid backbone. The pre-plasmid clone is designated MRKpAdHVE3+CMV(no intron)-FLgag-bGHpA and is 37,498 bp in size.

F. Virus Generation of an Enhanced Adenoviral Construct—“MRK Ad5 HIV-1gag”

MRK Ad5 HIV-1 gag contains the hCMV(no intron)-FLgag-bGHpA transgene inserted into the new E3+ adenovector backbone, MRKpAdHVE3, in the E1 parallel orientation. We have designated this adenovector MRK Ad5 HIV-1 gag. This construct was prepared as outlined below:

The pre-plasmid MRKpAdHVE3+CMV(no intron)-FLgag-bGHpA was digested with Pac1 to release the vector backbone and 3.3 μg was transfected by the calcium phosphate method (Amersham Pharmacia Biotech.) in a 6 cm dish containing PER.C6® cells at −60% confluence. Once CPE was reached (7-10 days), the culture was freeze/thawed three times and the cell debris pelleted. 1 ml of this cell lysate was used to infect into a 6 cm dish containing PER.C6® cells at 80-90% confluence. Once CPE was reached, the culture was freeze/thawed three times and the cell debris pelleted. The cell lysate was then used to infect a 15 cm dish containing PER.C6® cells at 80-90% confluence. This infection procedure was continued and expanded at passage 6. The virus was then extracted from the cell pellet by CsCl method. Two bandings were performed (3-gradient CsCl followed by a continuous CsCl gradient). Following the second banding, the virus was dialyzed in A105 buffer. Viral DNA was extracted using pronase treatment followed by phenol chloroform. The viral DNA was then digested with HindIII and radioactively labeled with [³³P]dATP. Following gel electrophoresis to separate the digestion products the gel was dried down on Whatman paper and then subjected to autoradiography. The digestion products were compared with the digestion products from the pre-plasmid (that had been digested with Pac1/HindIII prior to labeling). The expected sizes were observed, indicating that the virus had been successfully rescued.

### EXAMPLE 3

### Generation of Adenoviral Serotype 6 Vector Constructs

A. Construction of Ad6 Pre-Adenovirus Plasmid

An Ad6 based pre-adenovirus plasmid which could be used to generate first generation Ad6 vectors was constructed taking advantage of the extensive sequence homology (approx. 98%) between Ad5 and Ad6. Homologous recombination was used to clone wtAd6 sequences into a bacterial plasmid.

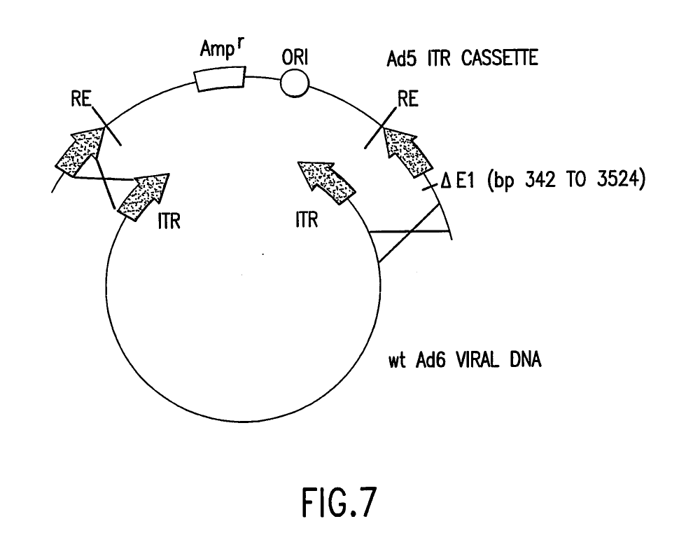

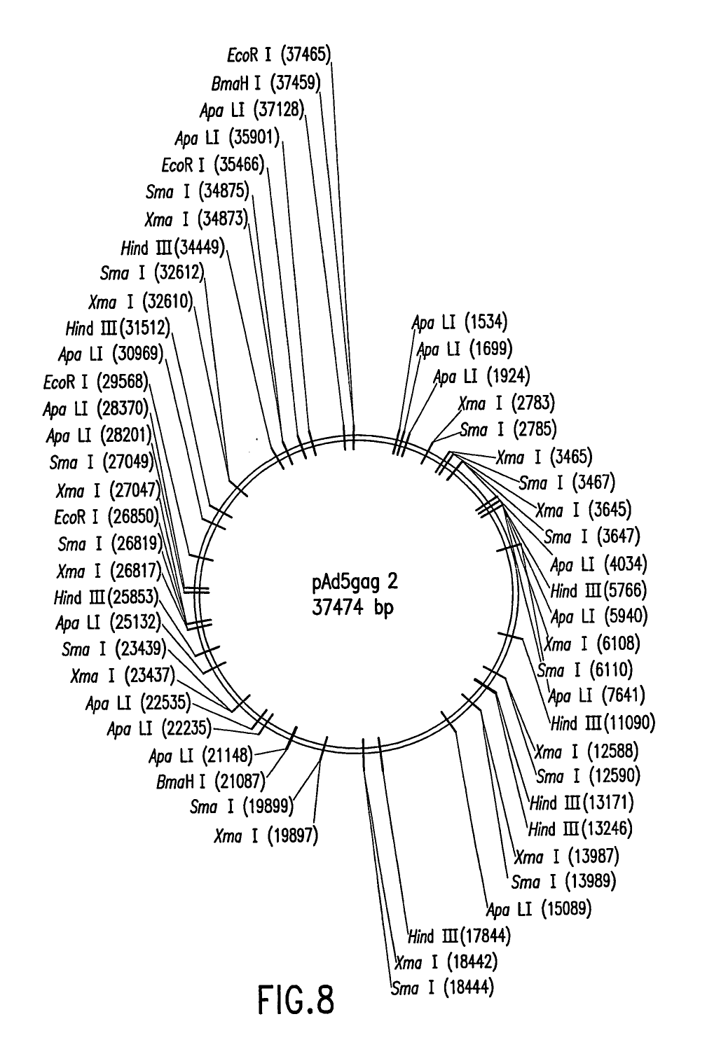

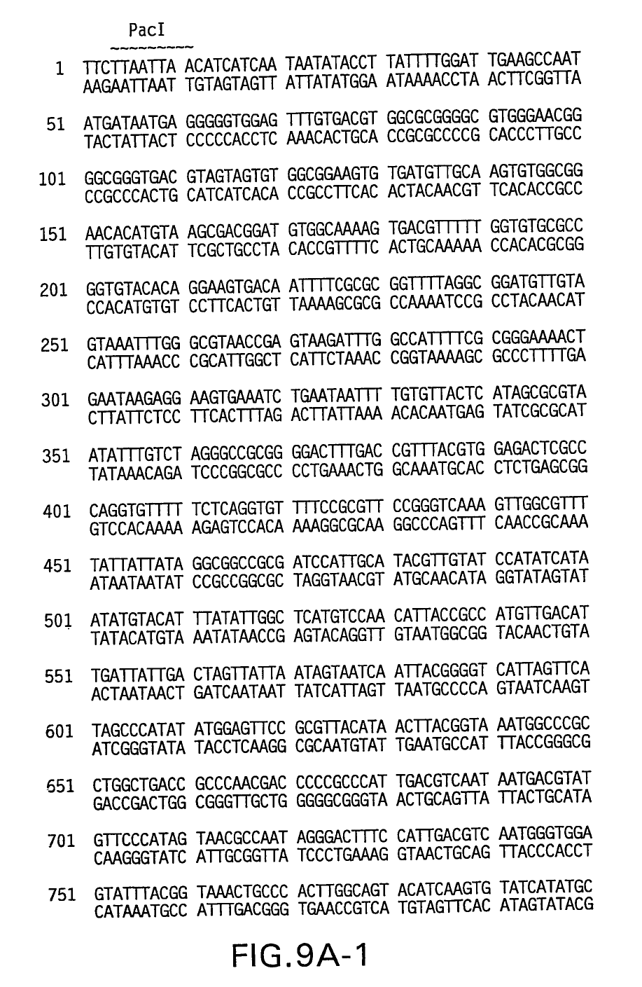

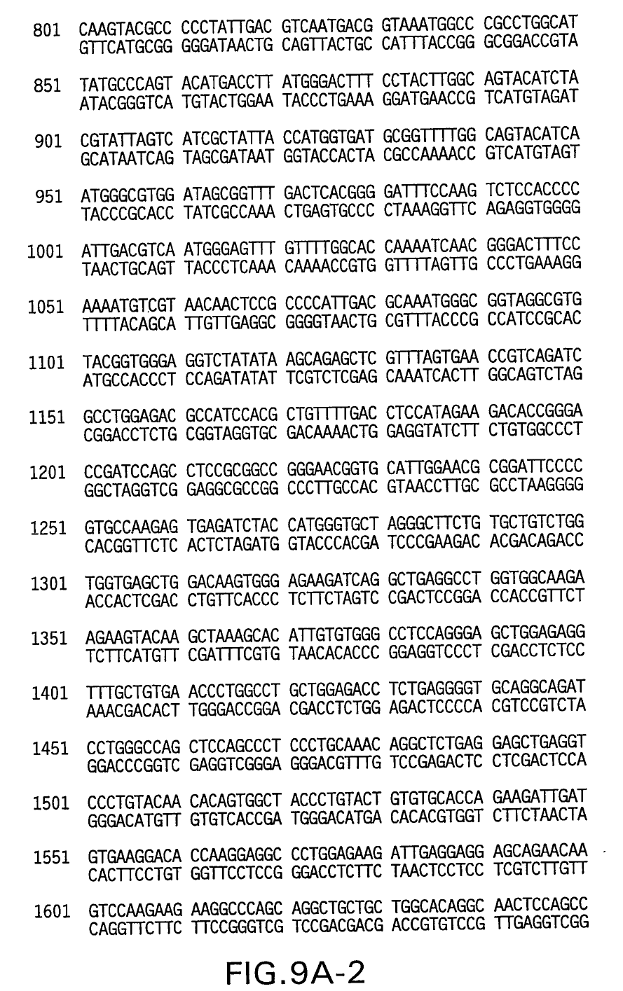

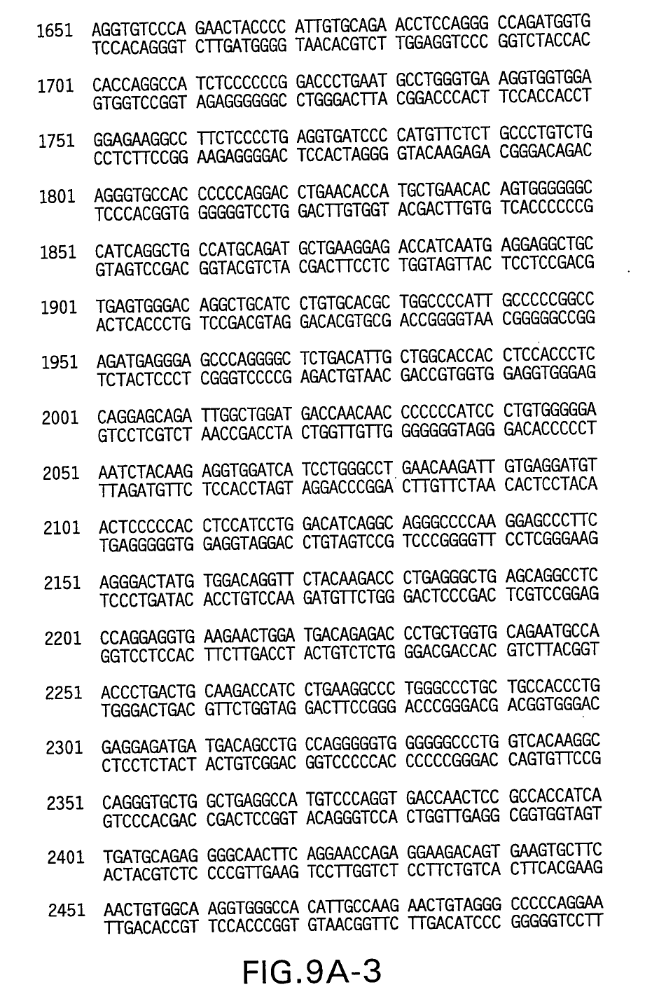

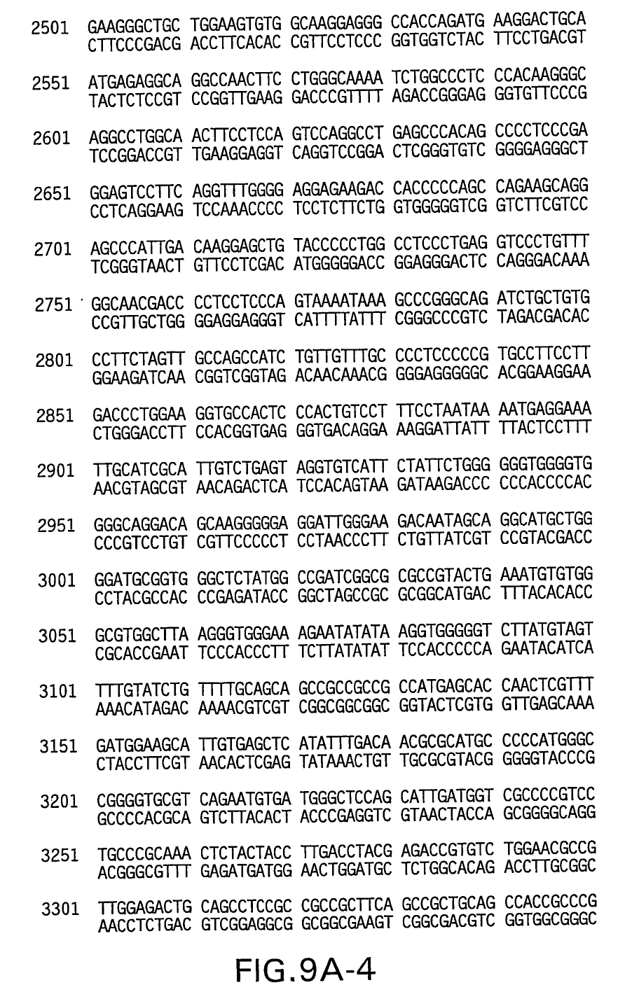

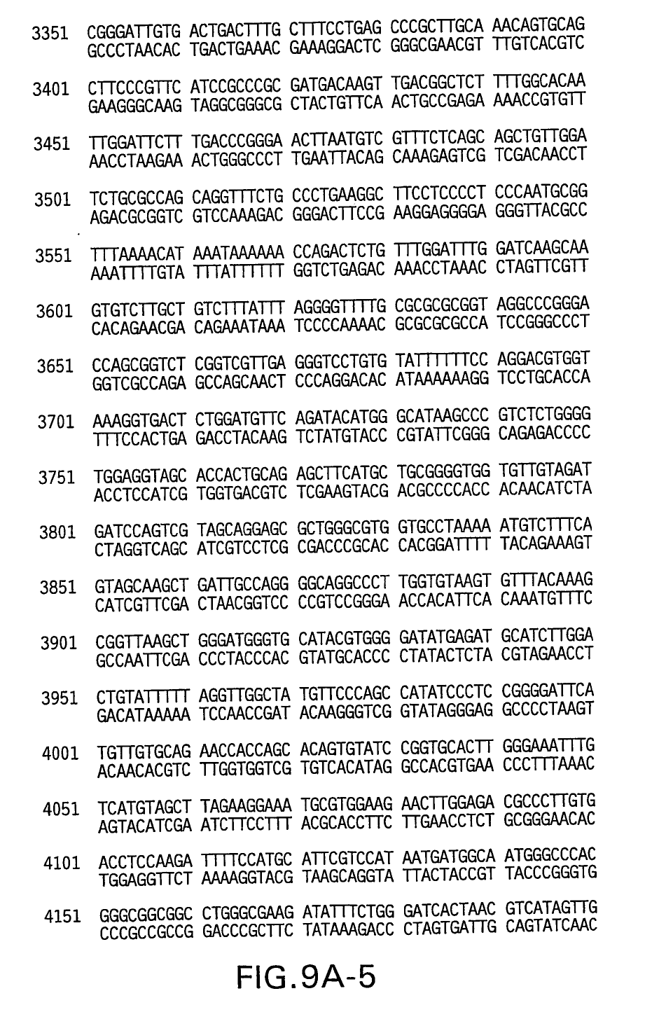

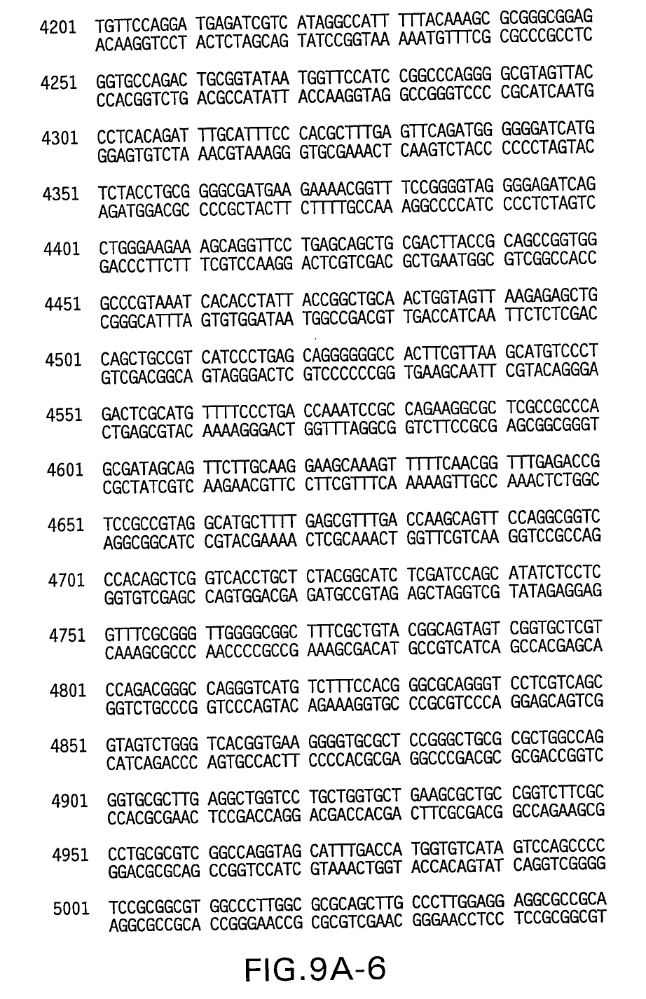

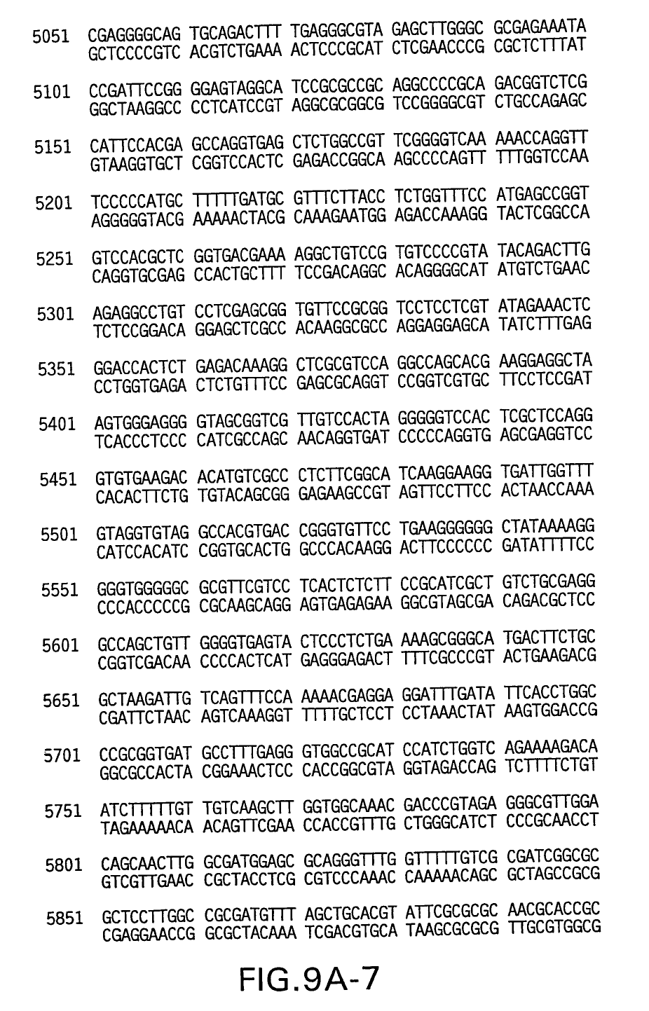

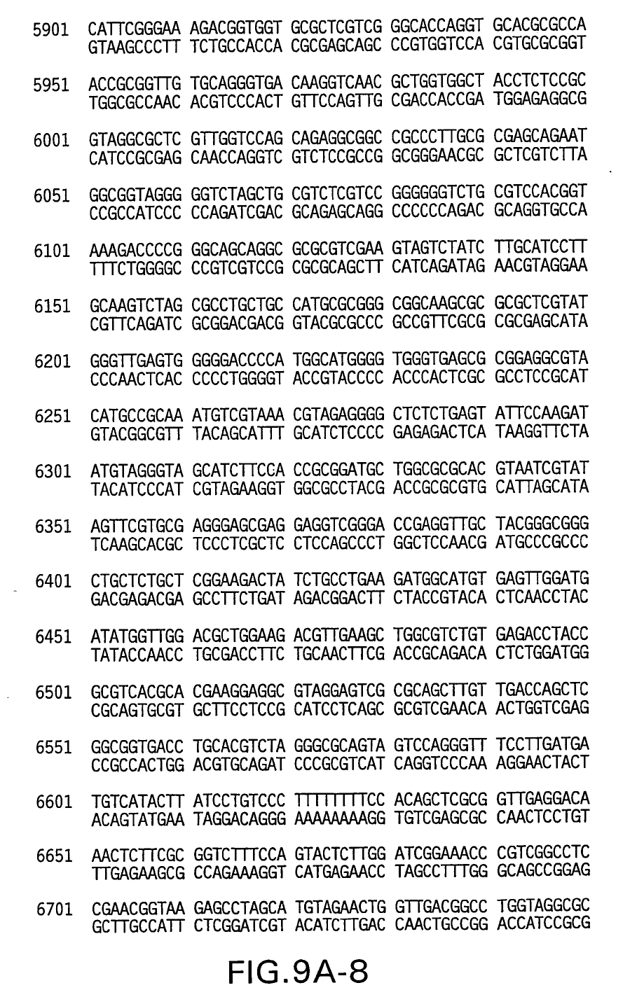

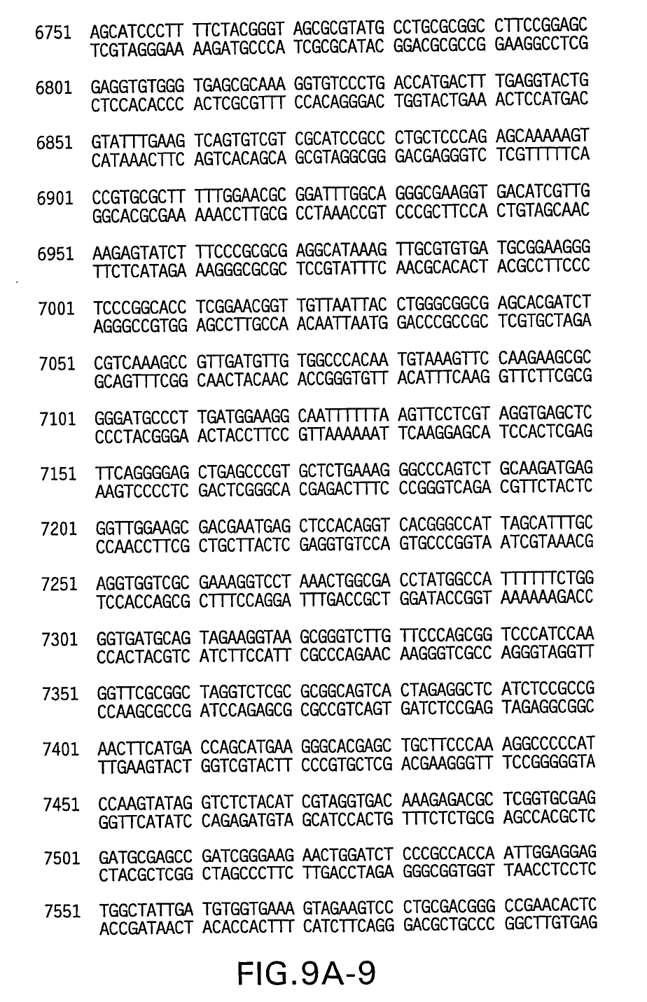

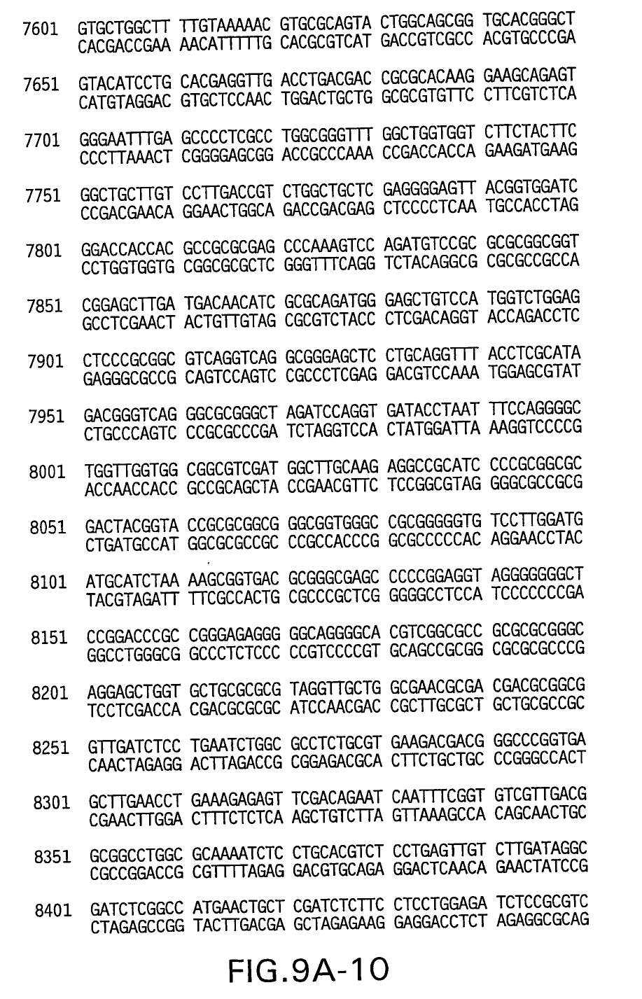

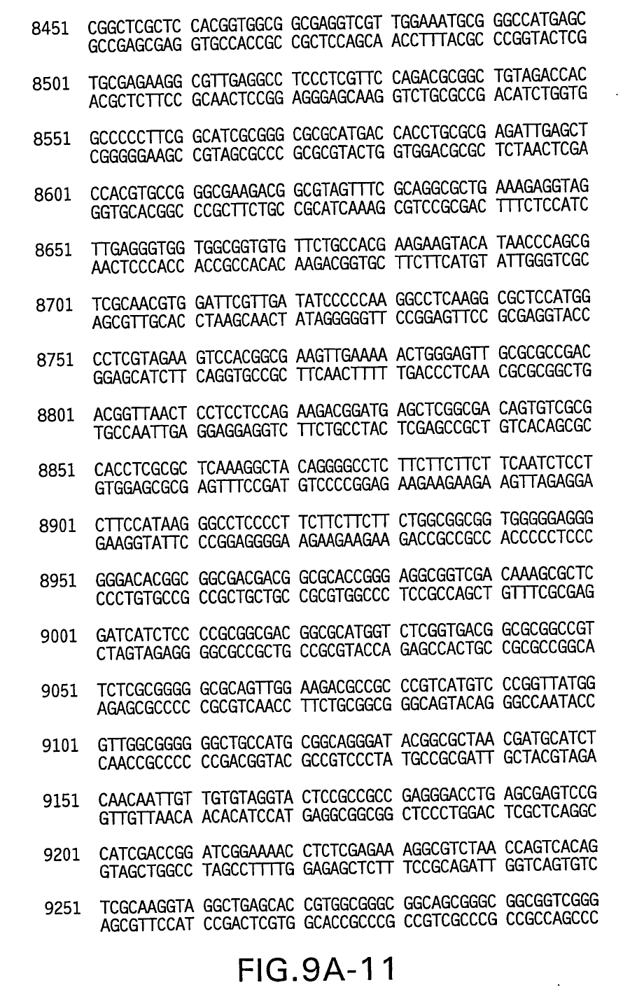

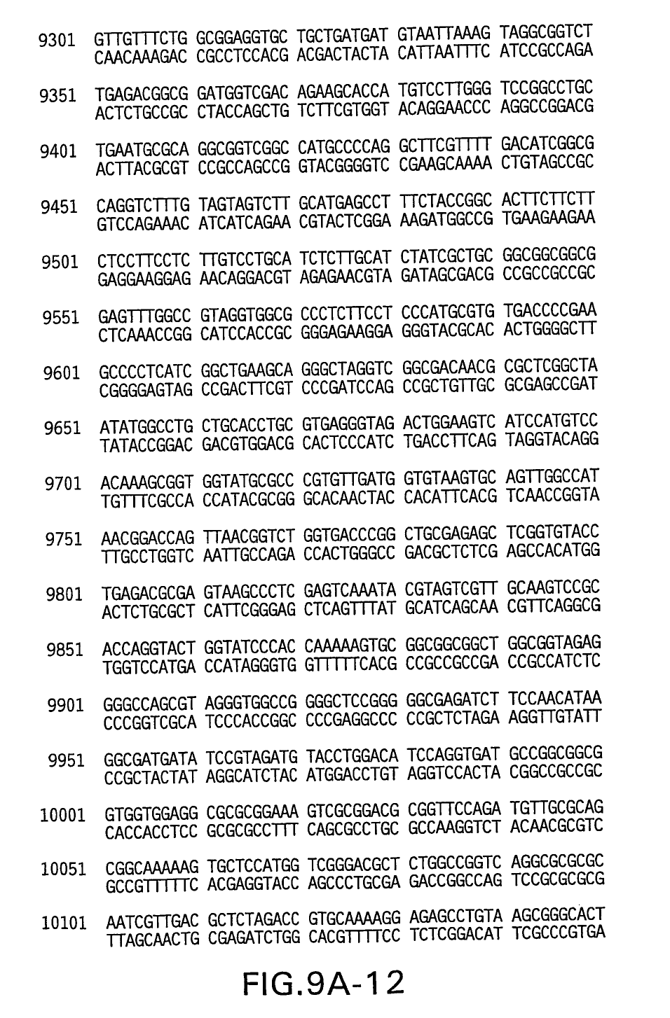

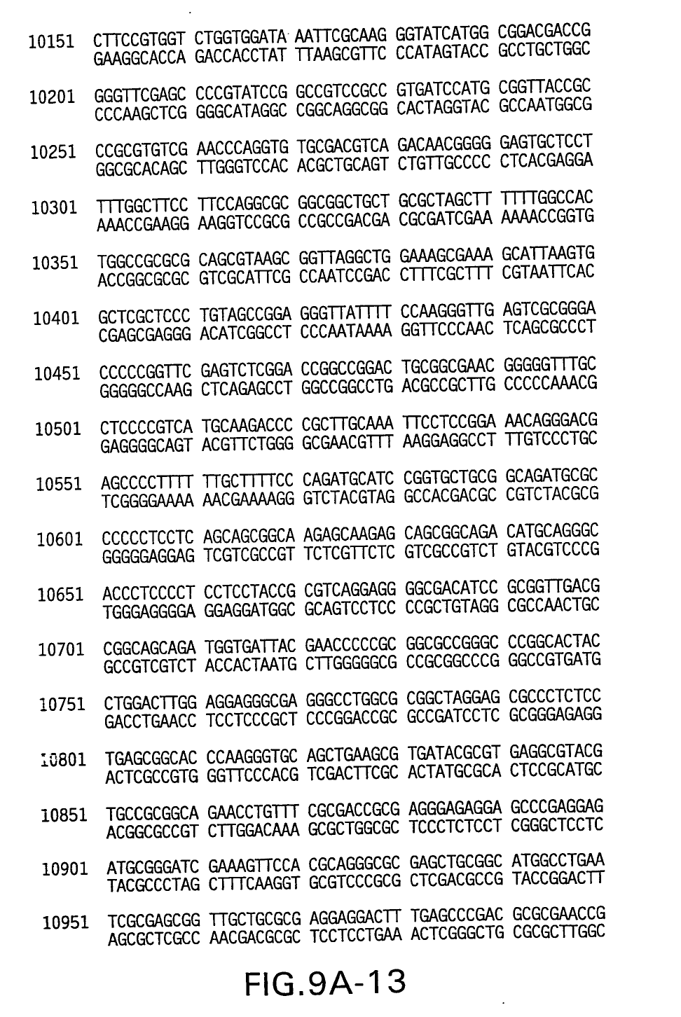

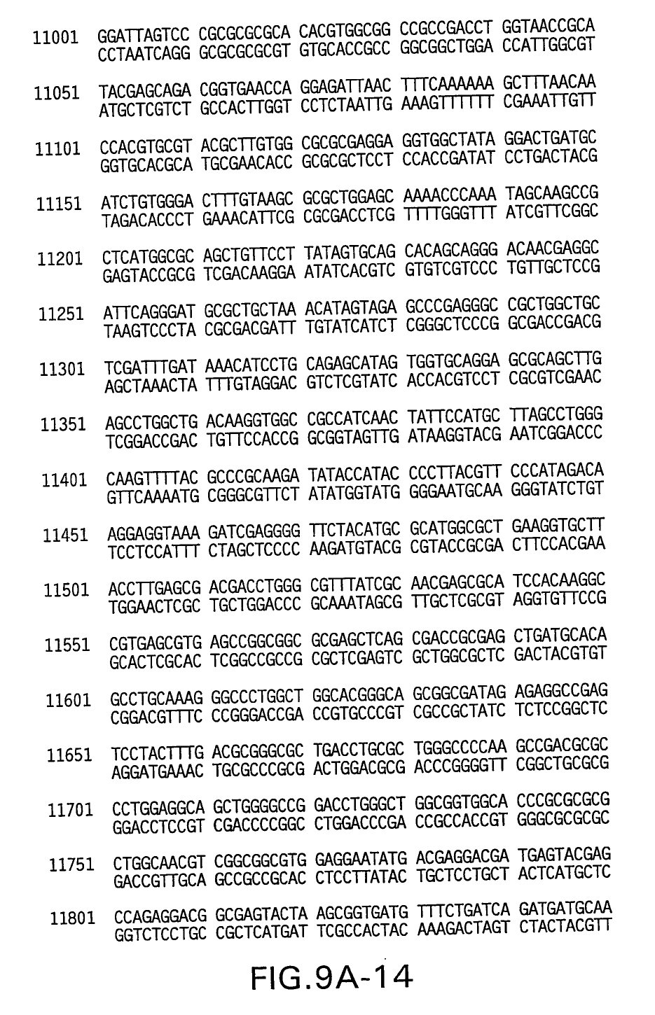

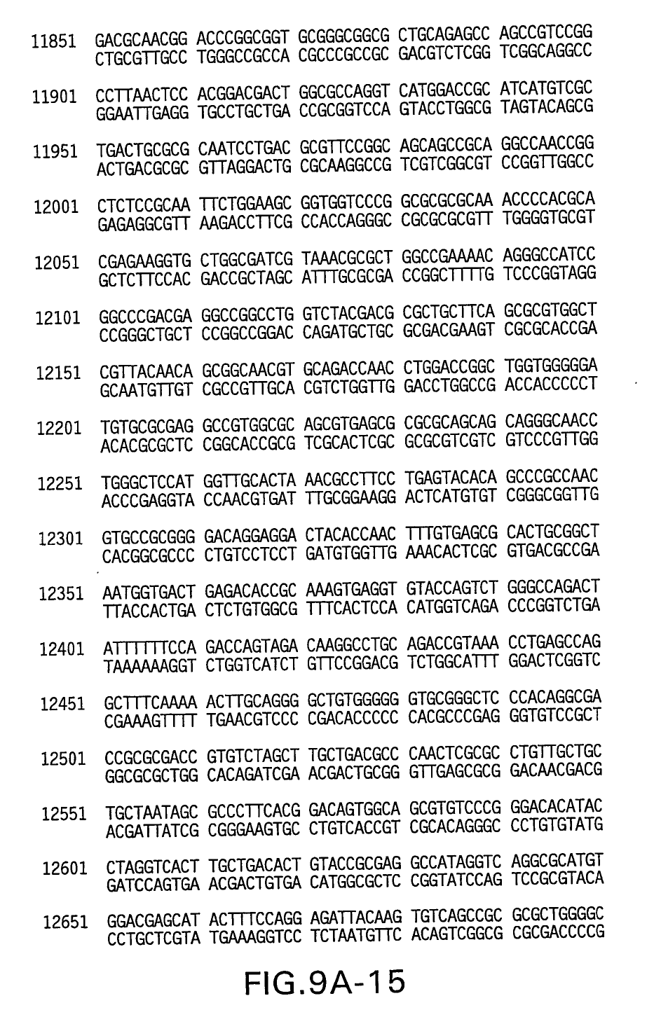

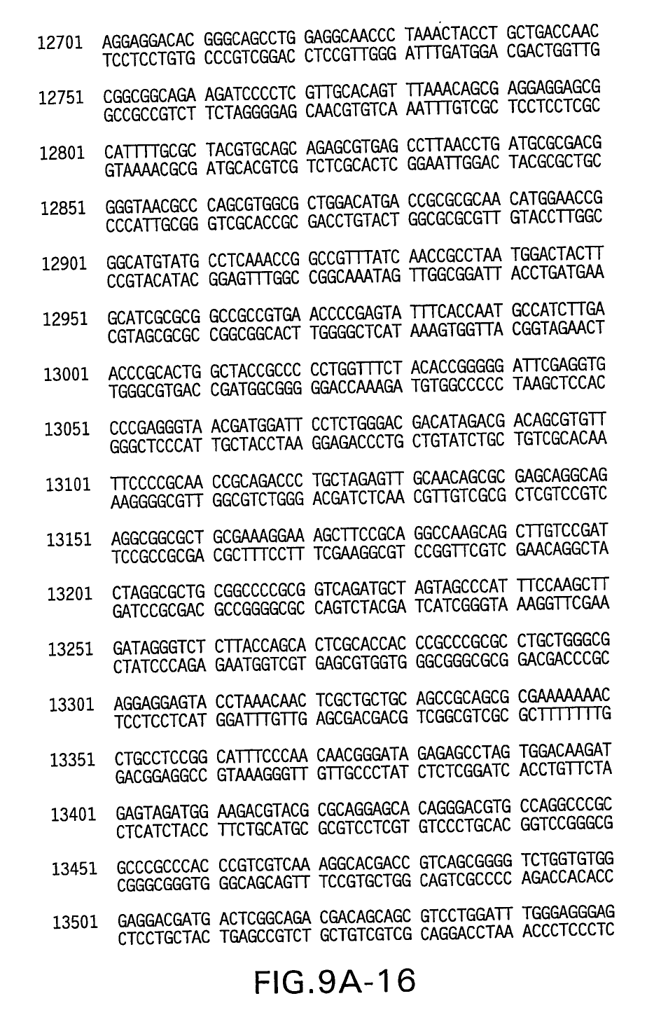

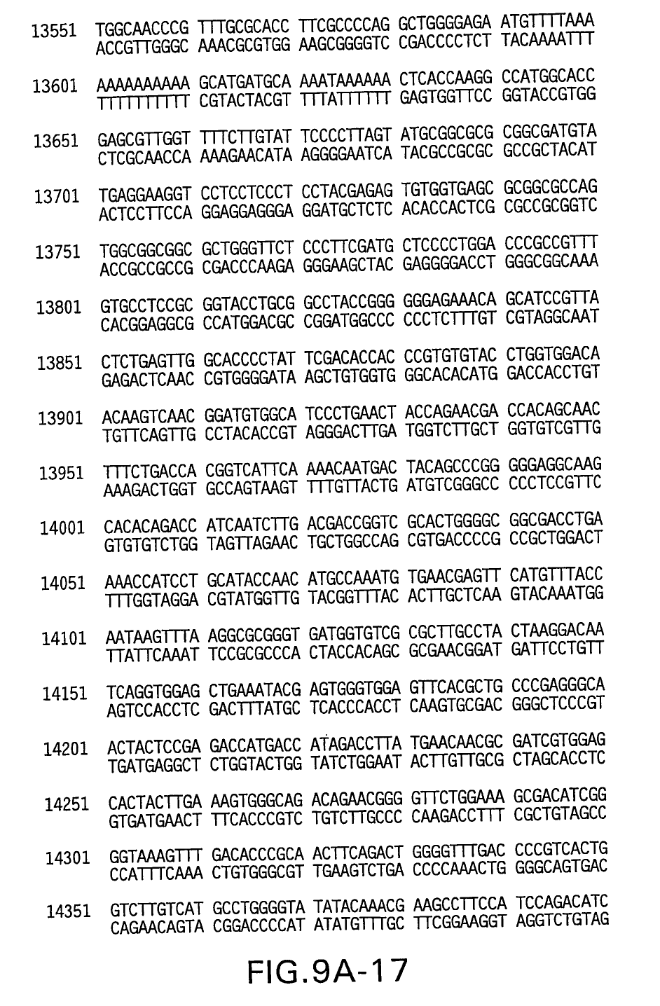

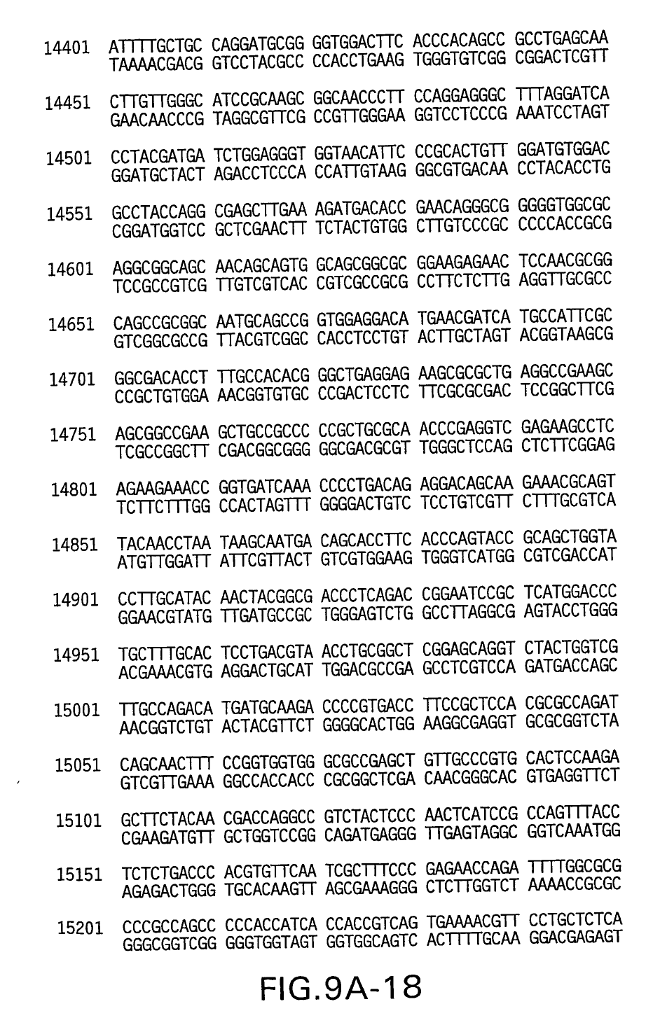

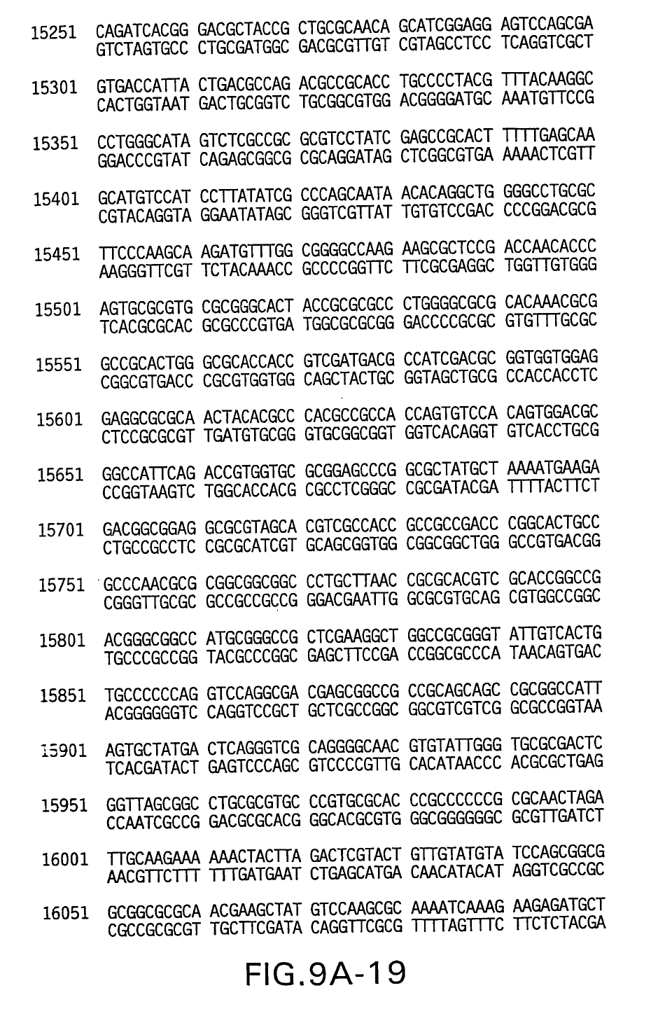

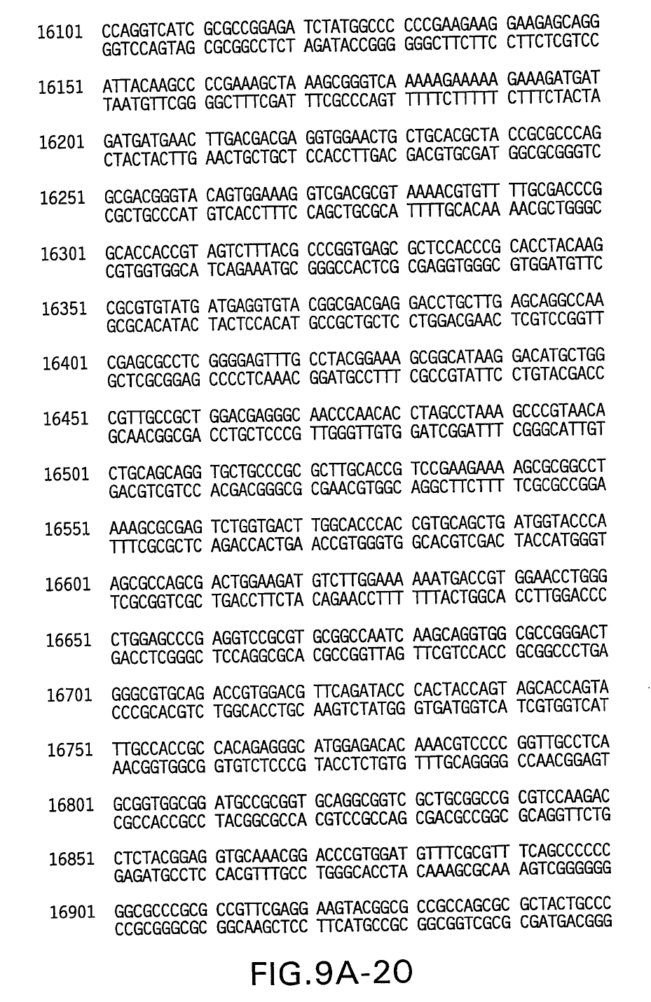

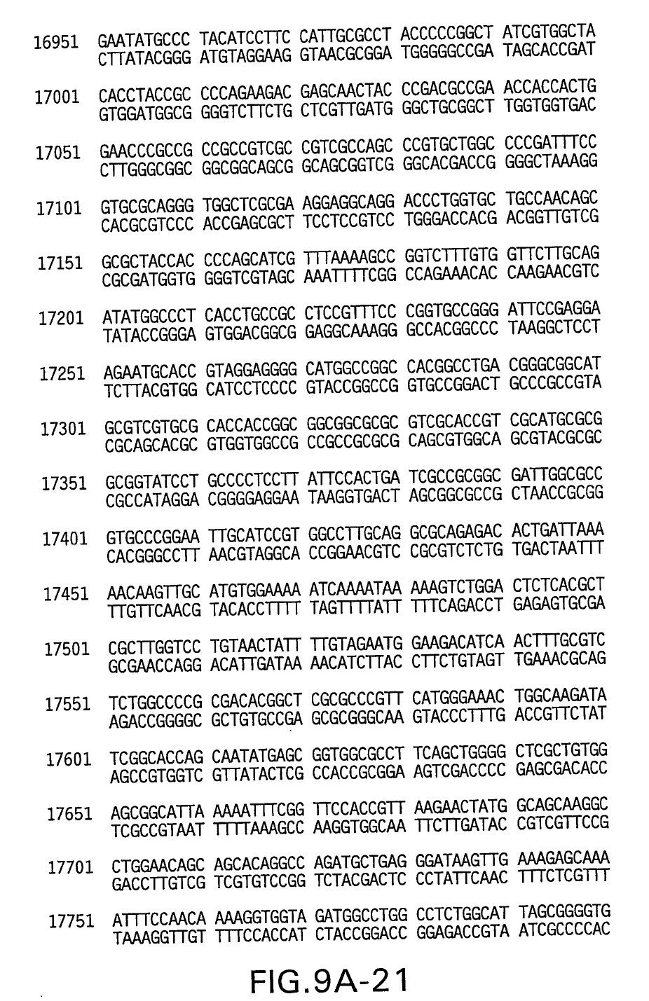

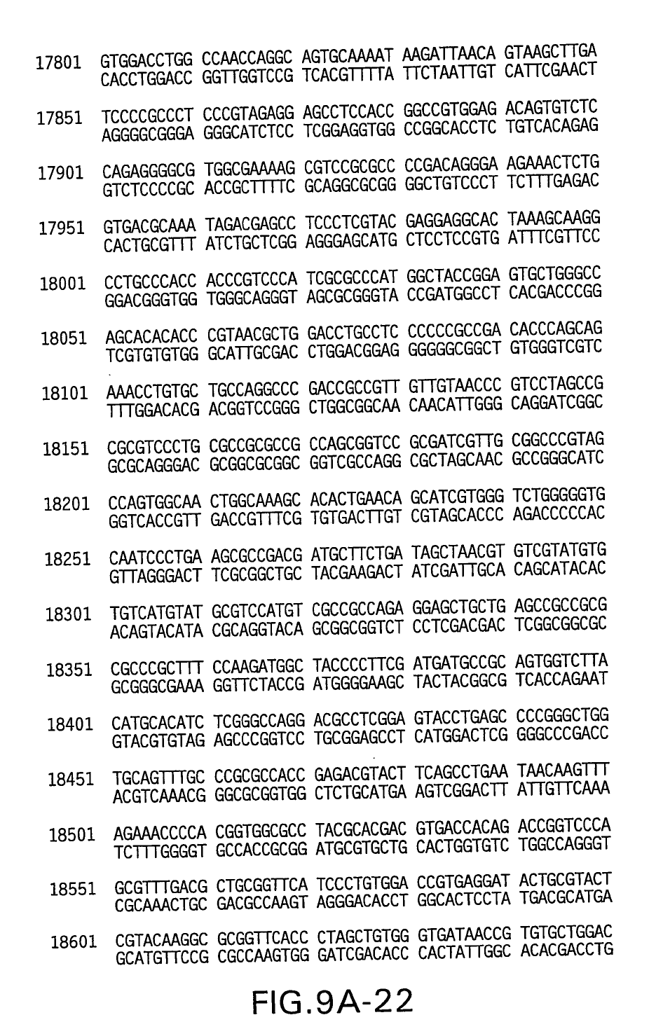

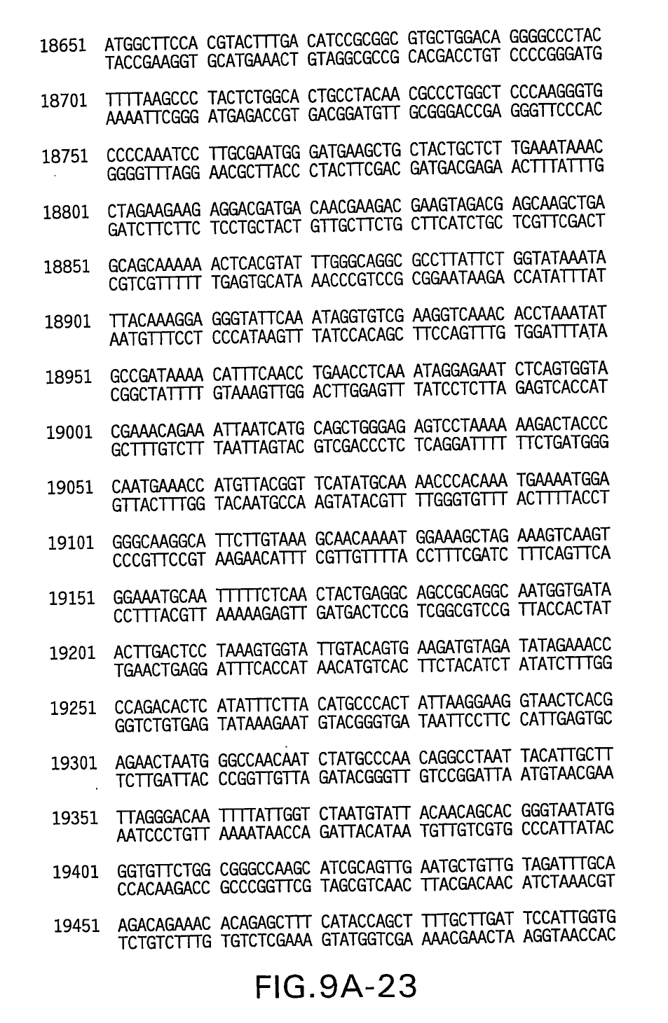

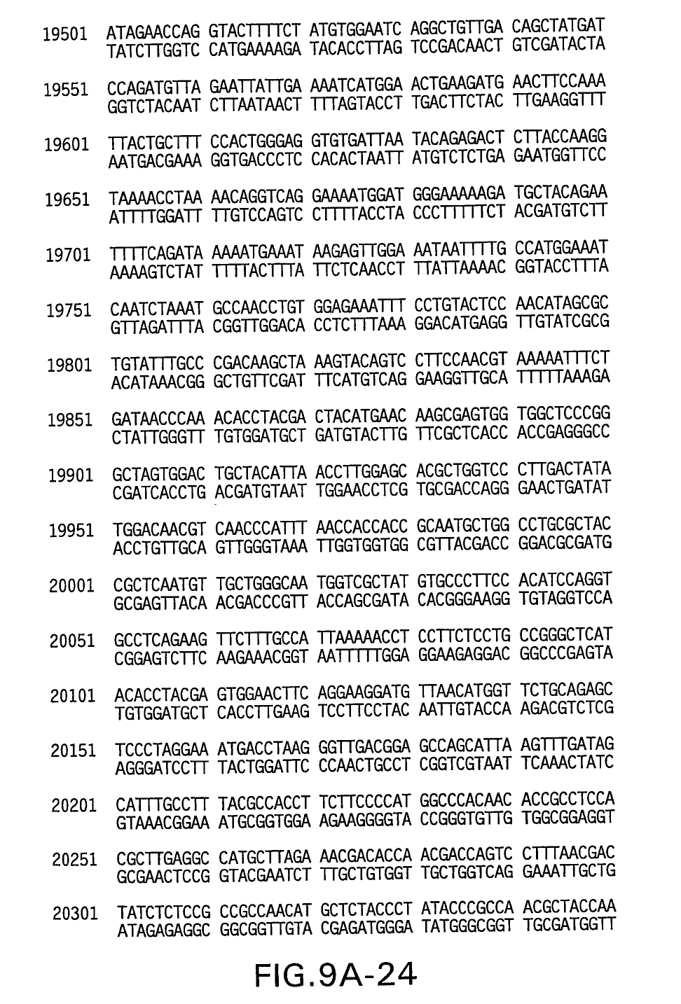

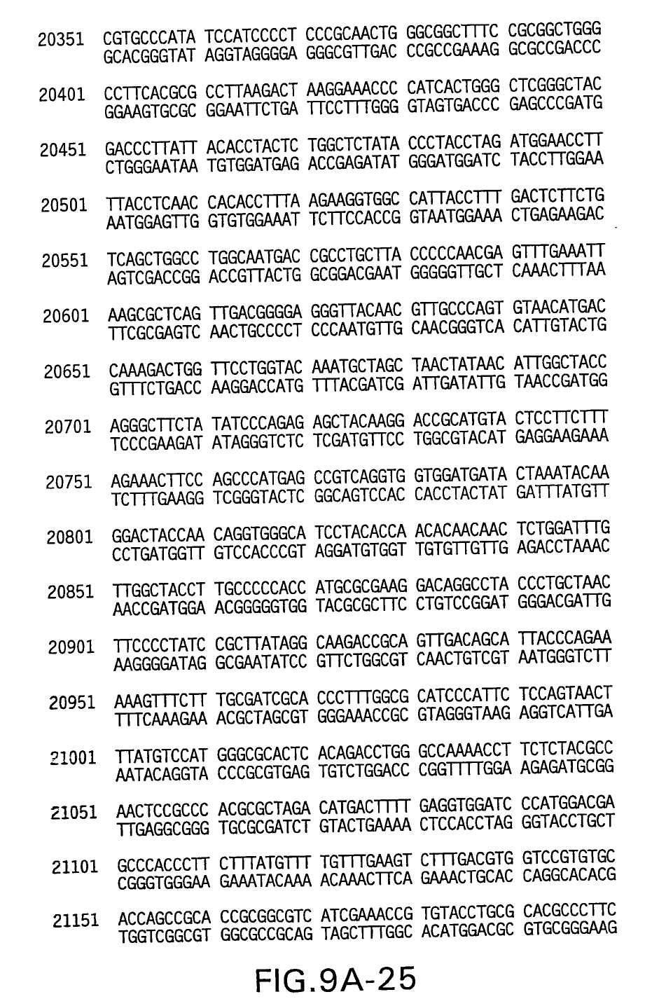

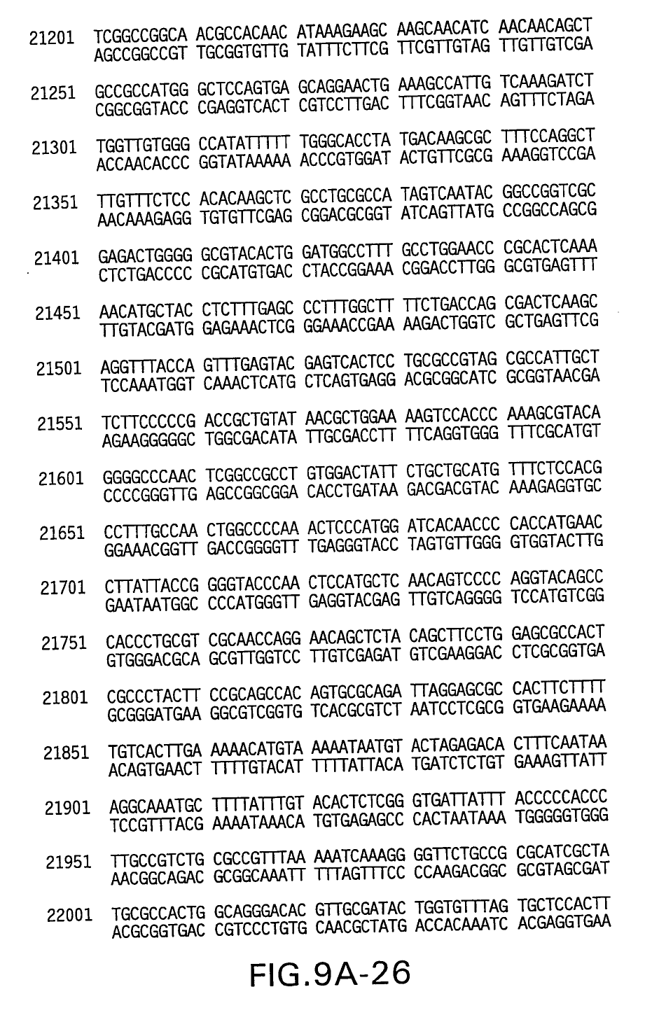

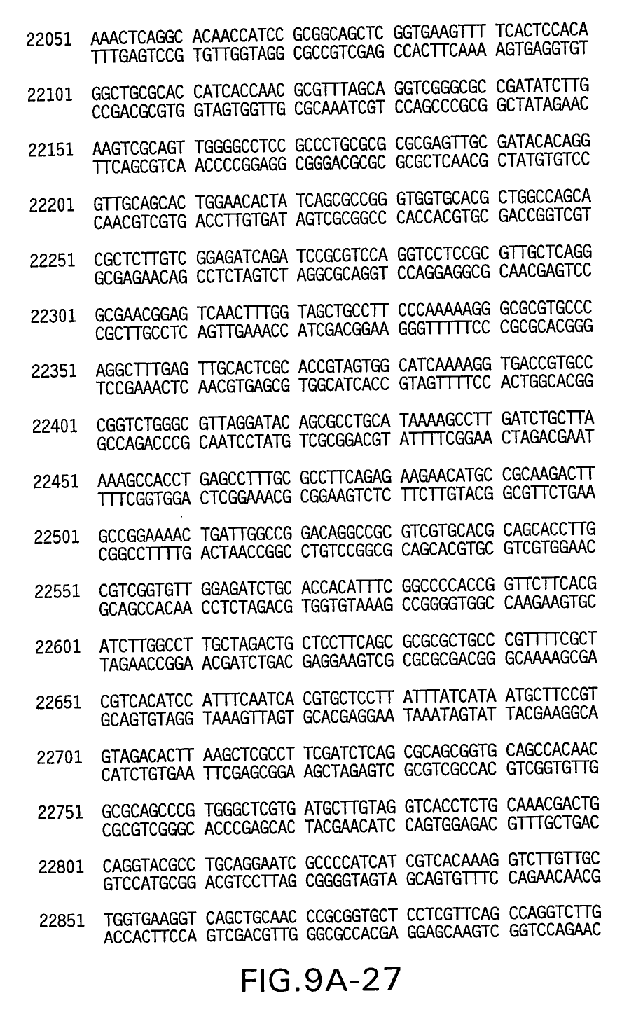

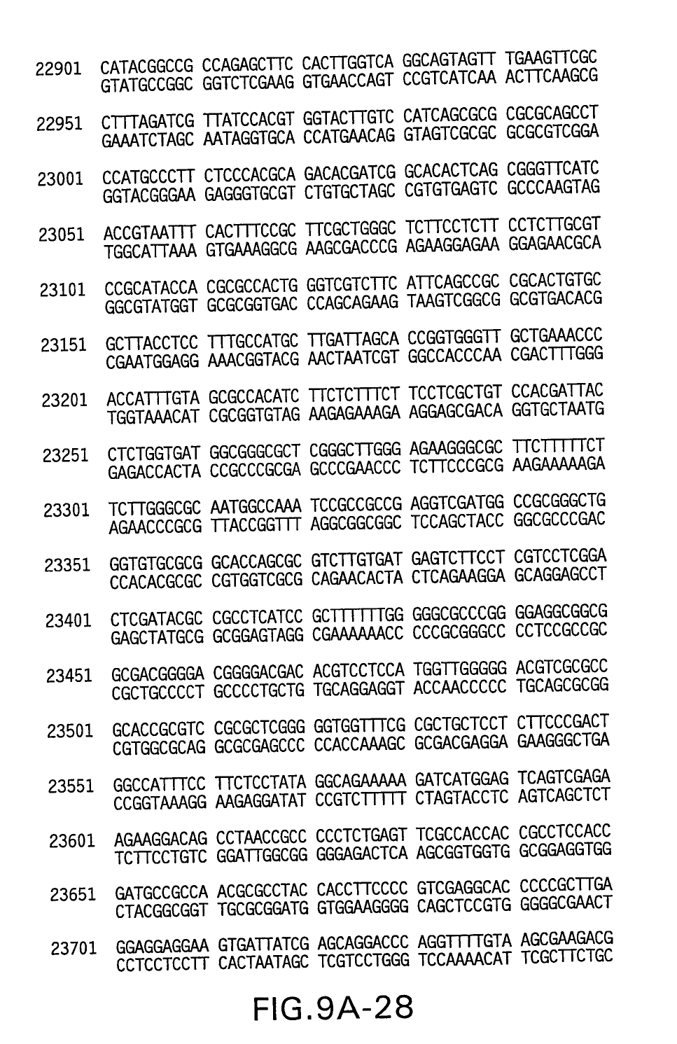

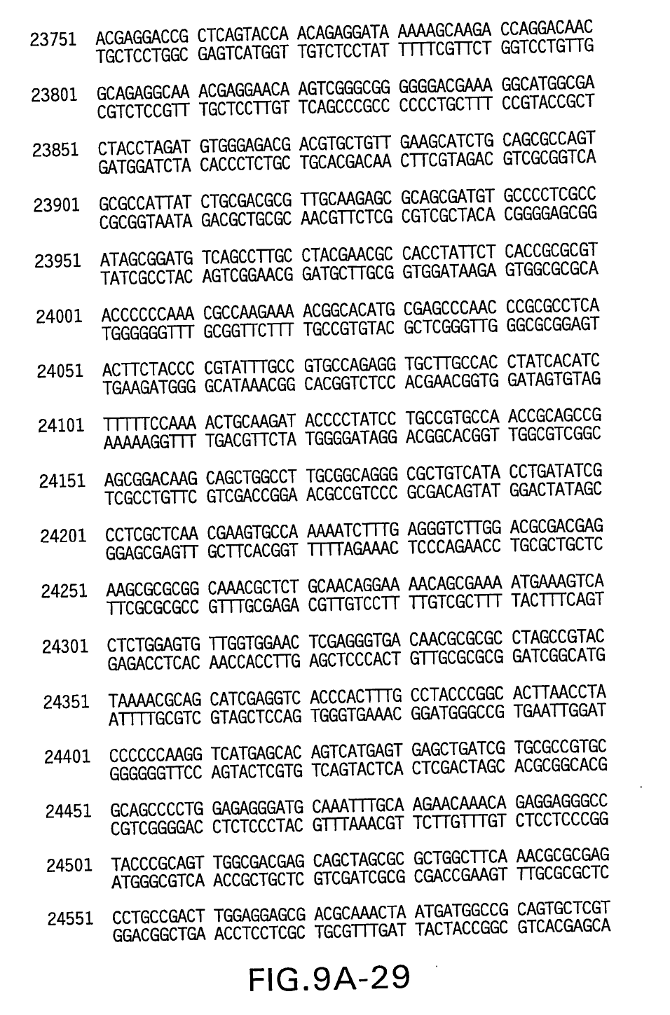

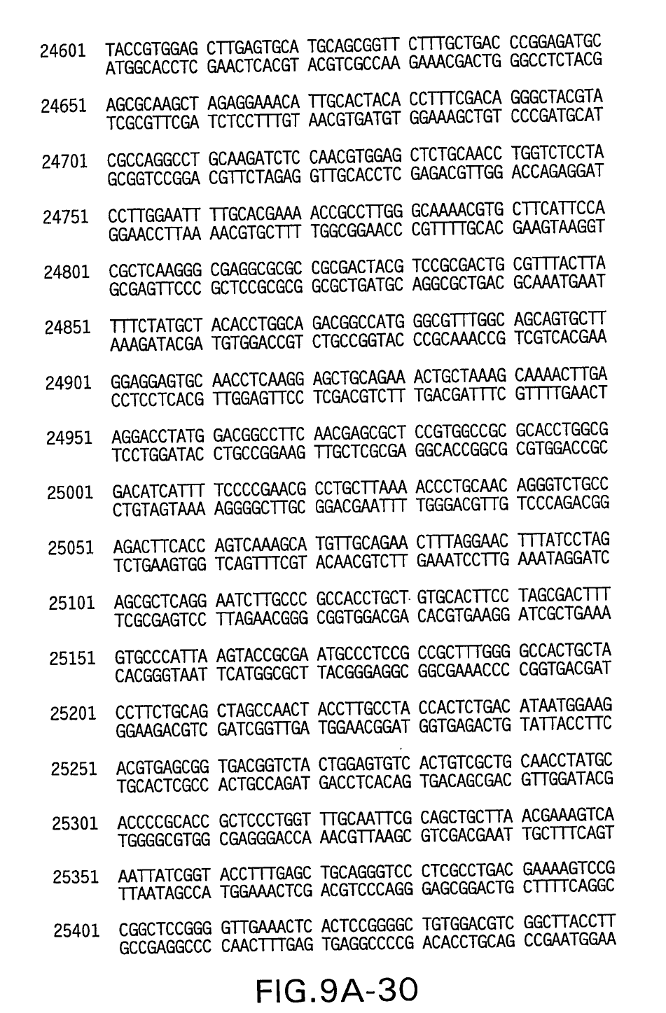

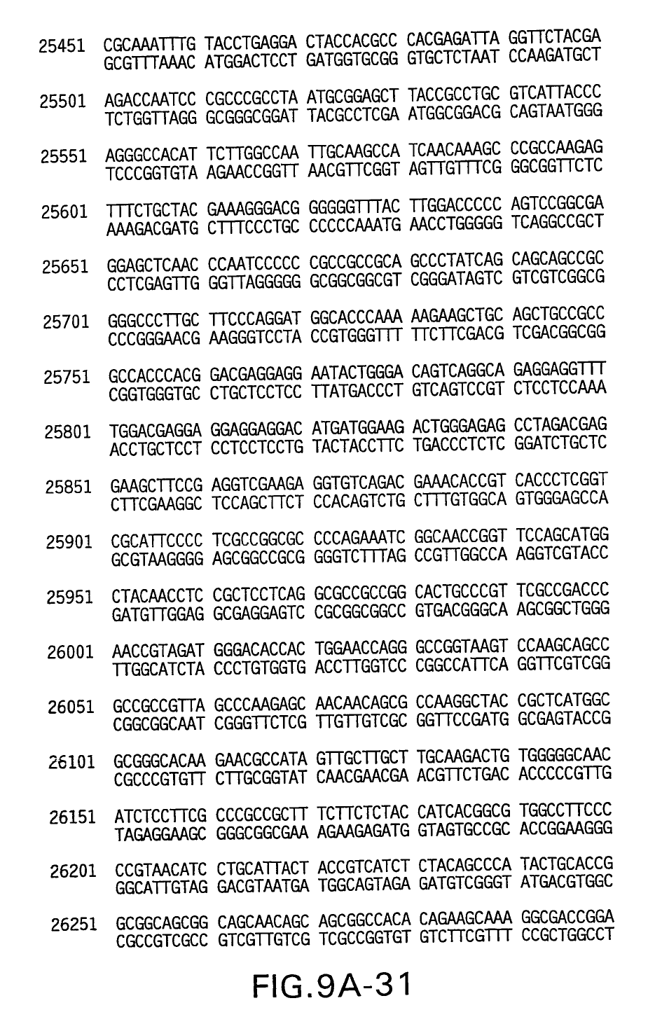

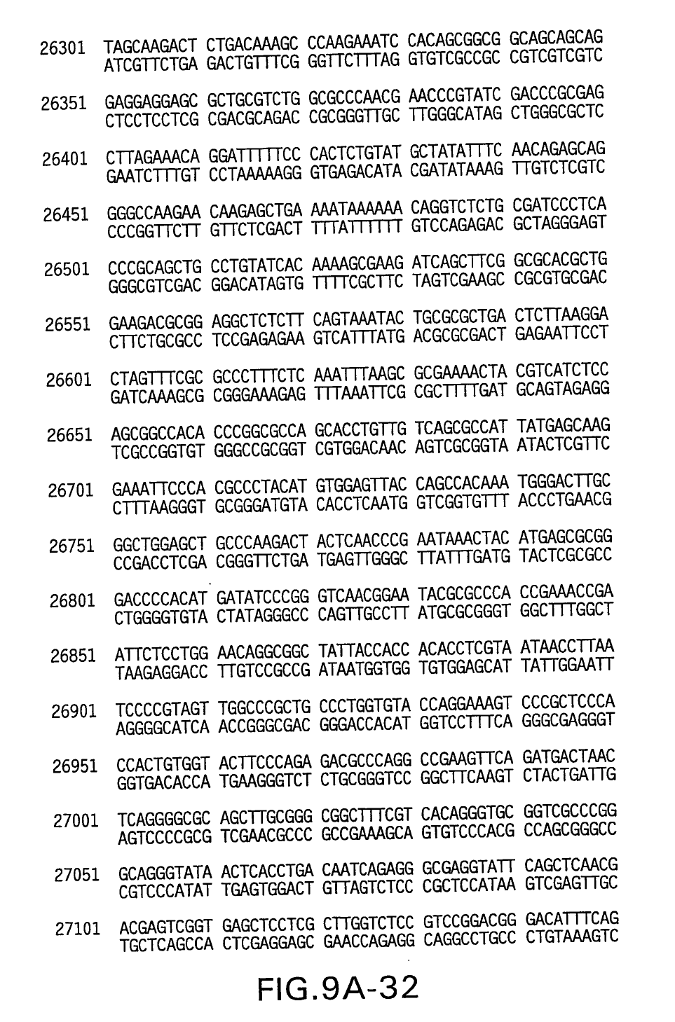

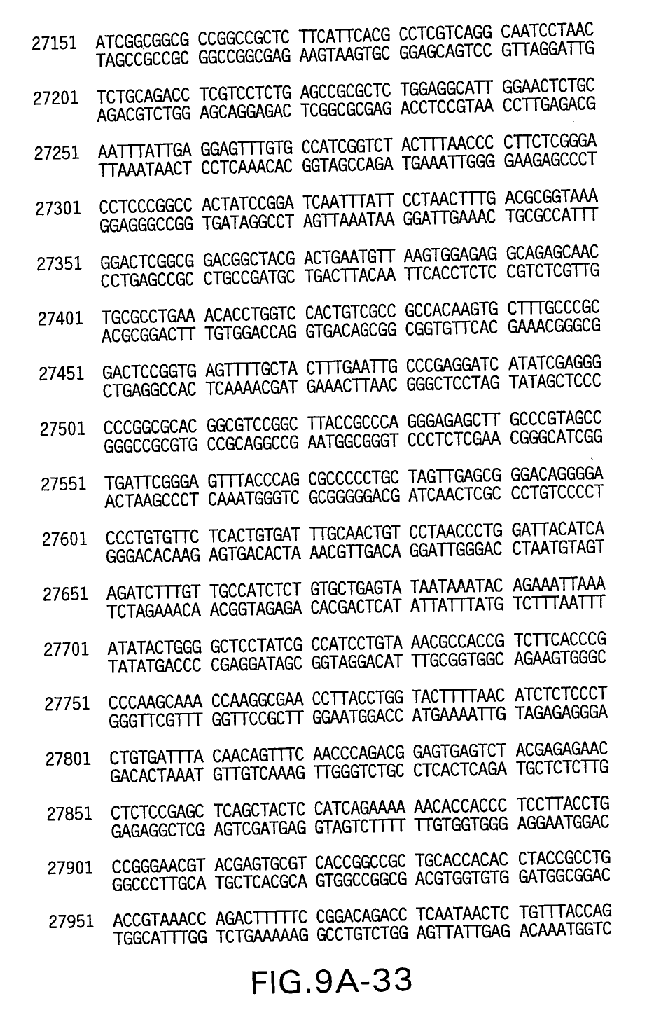

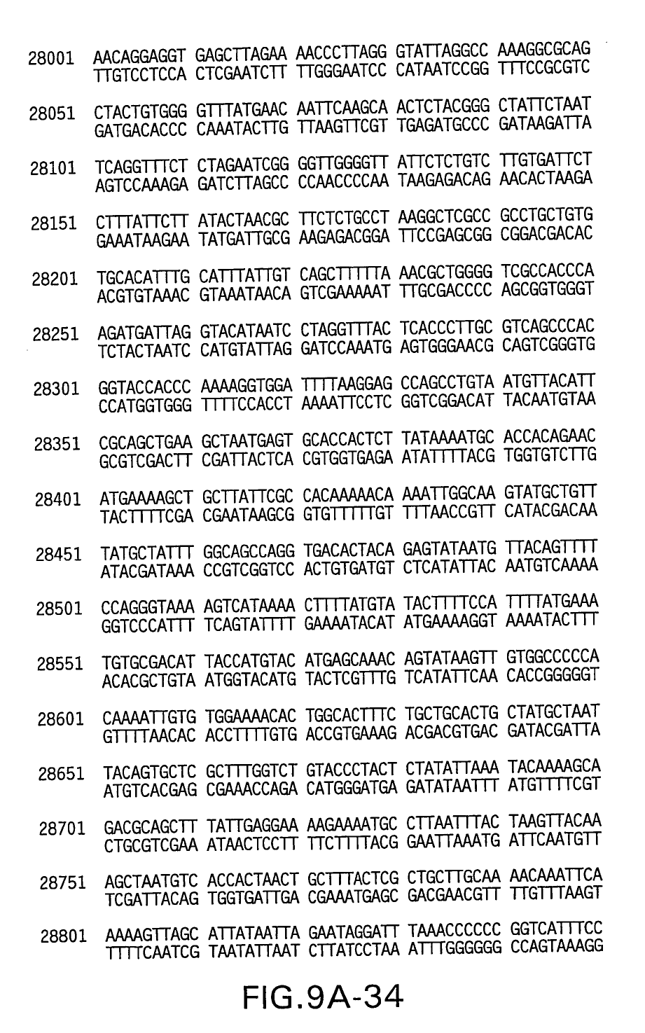

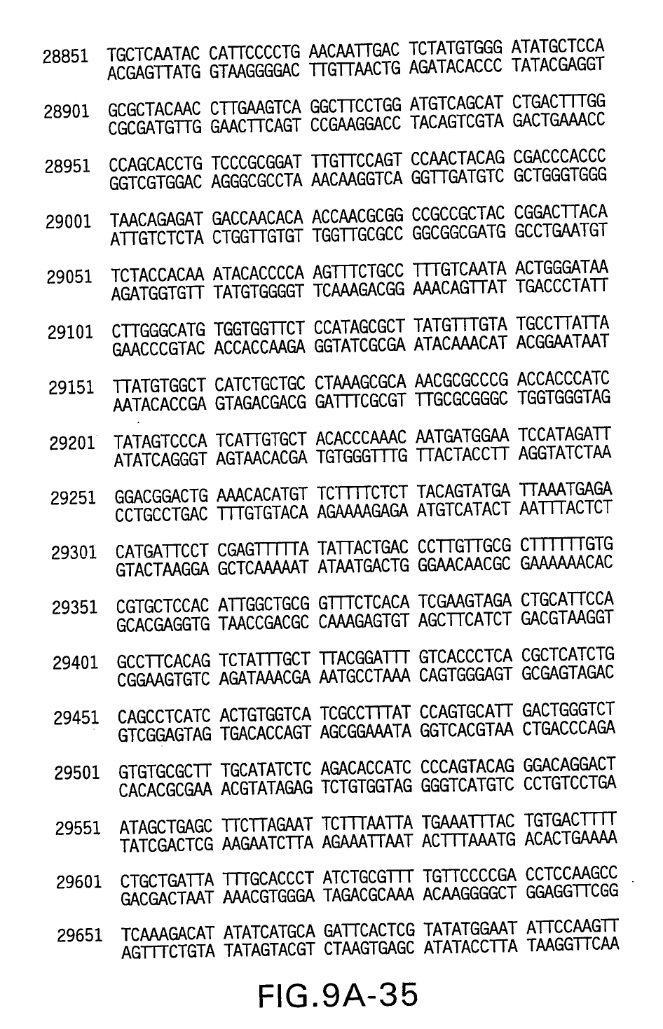

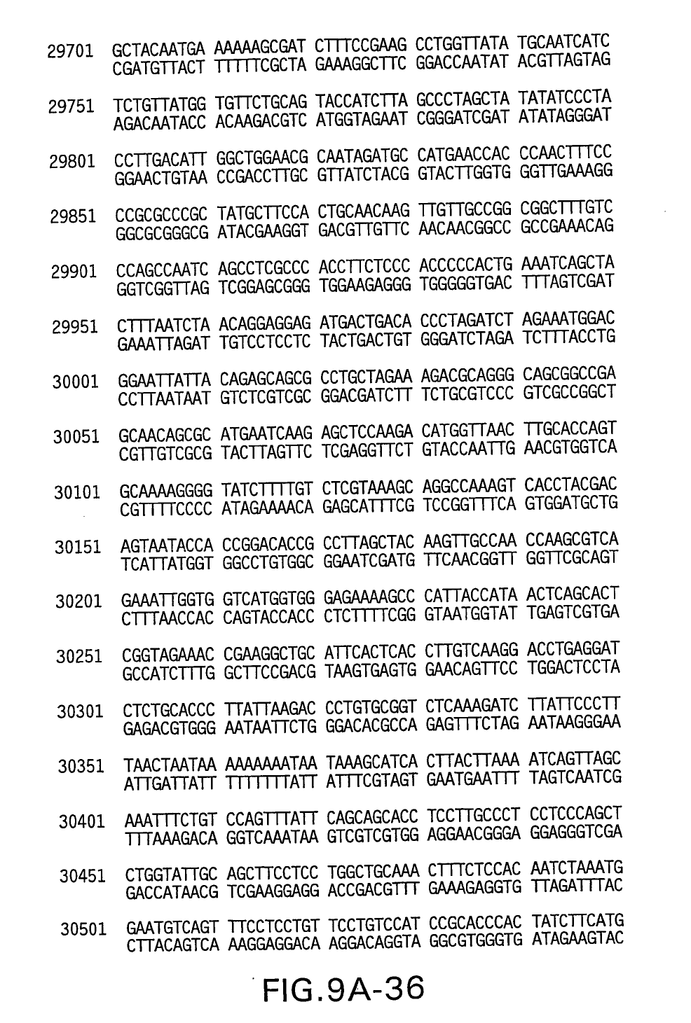

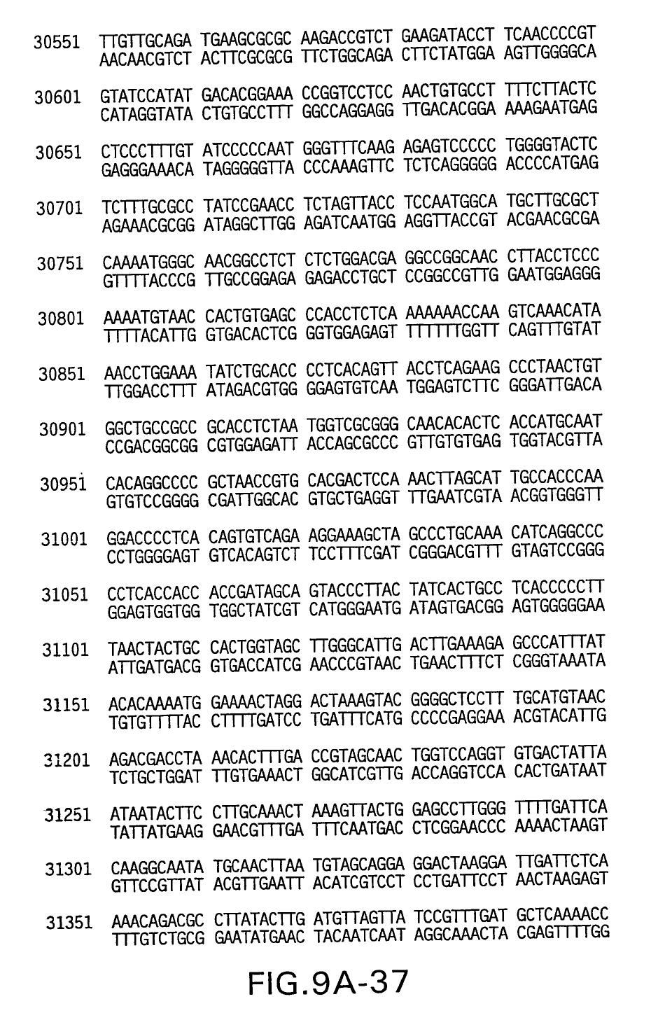

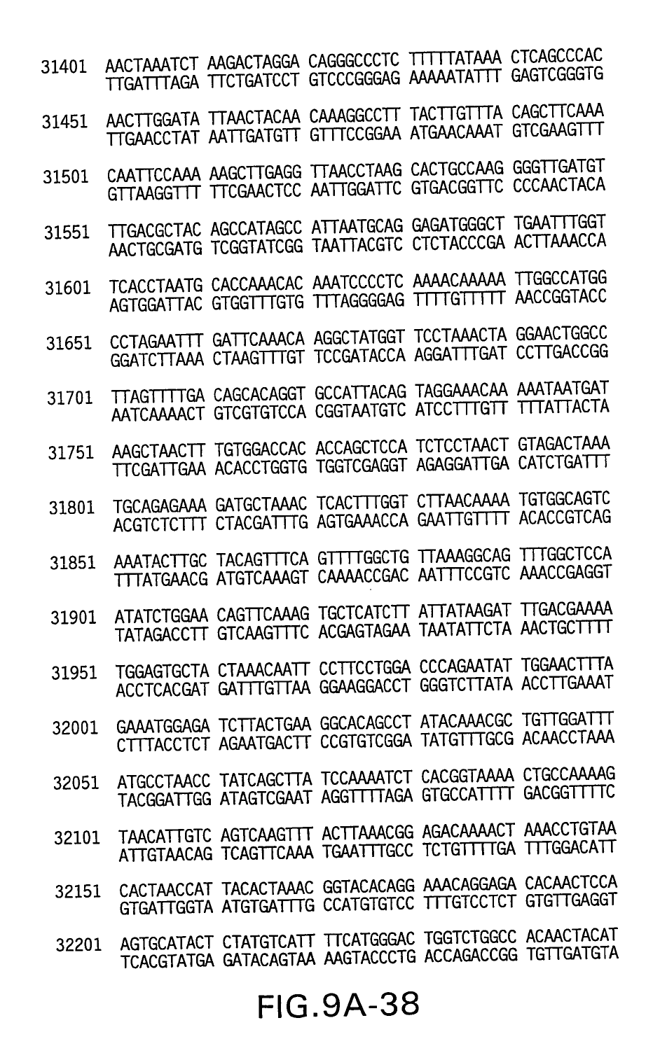

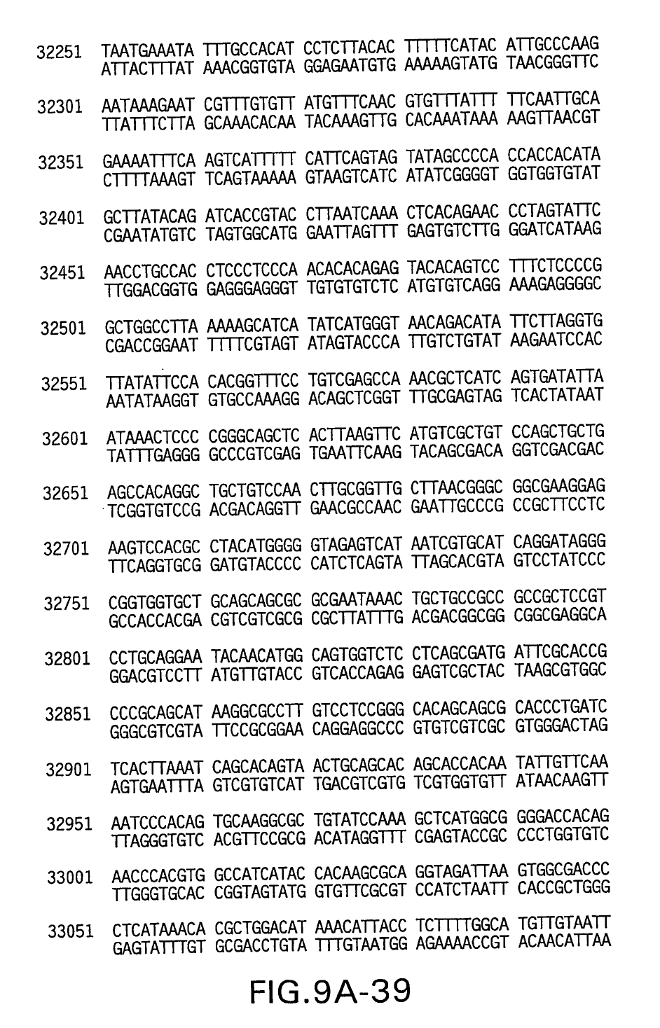

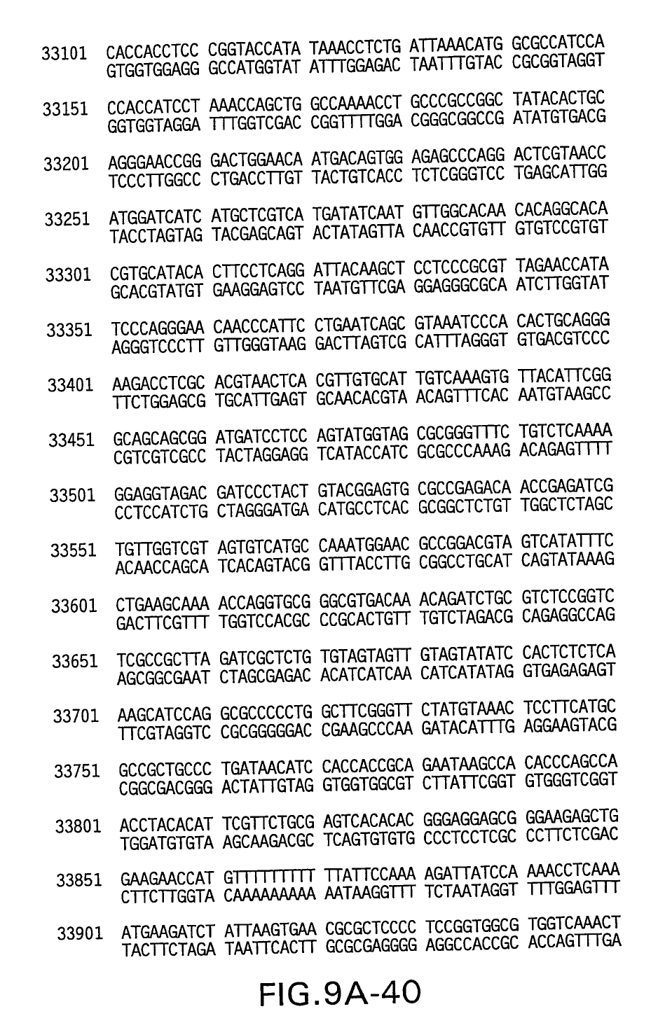

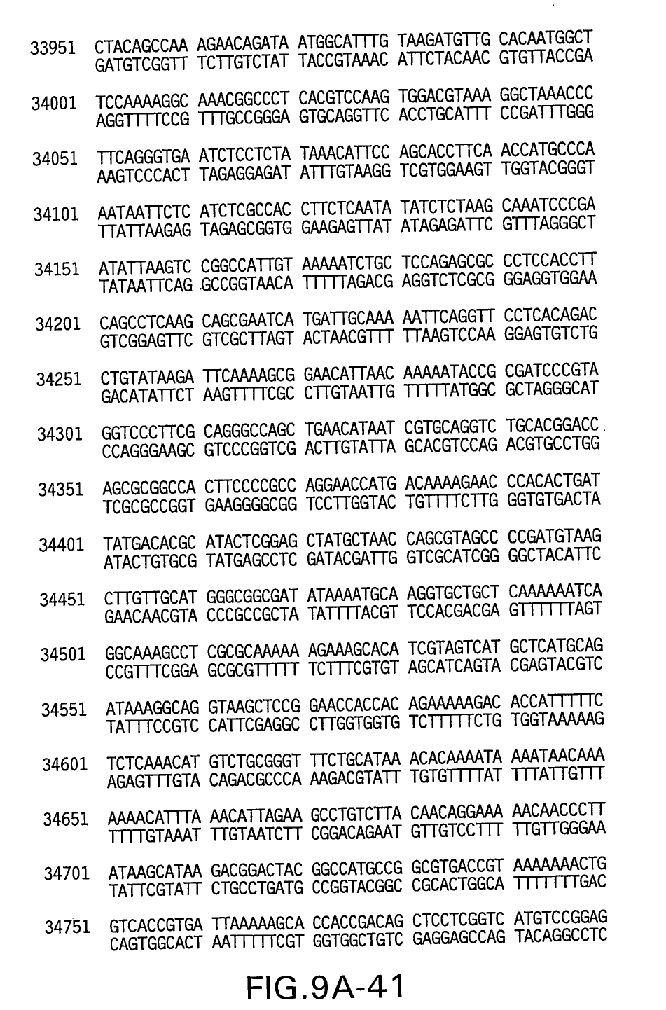

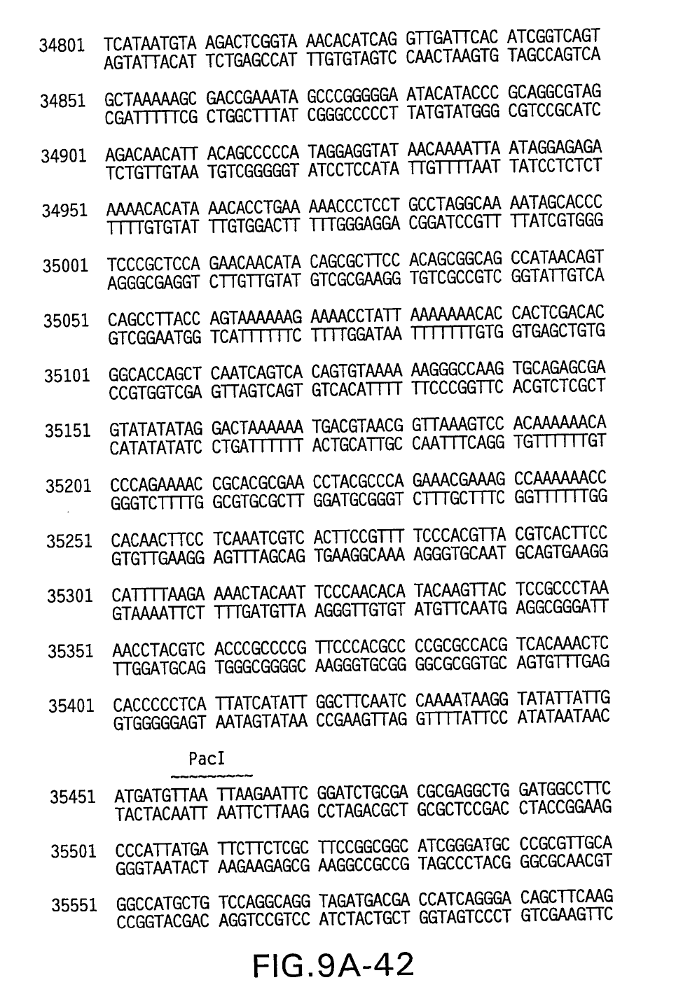

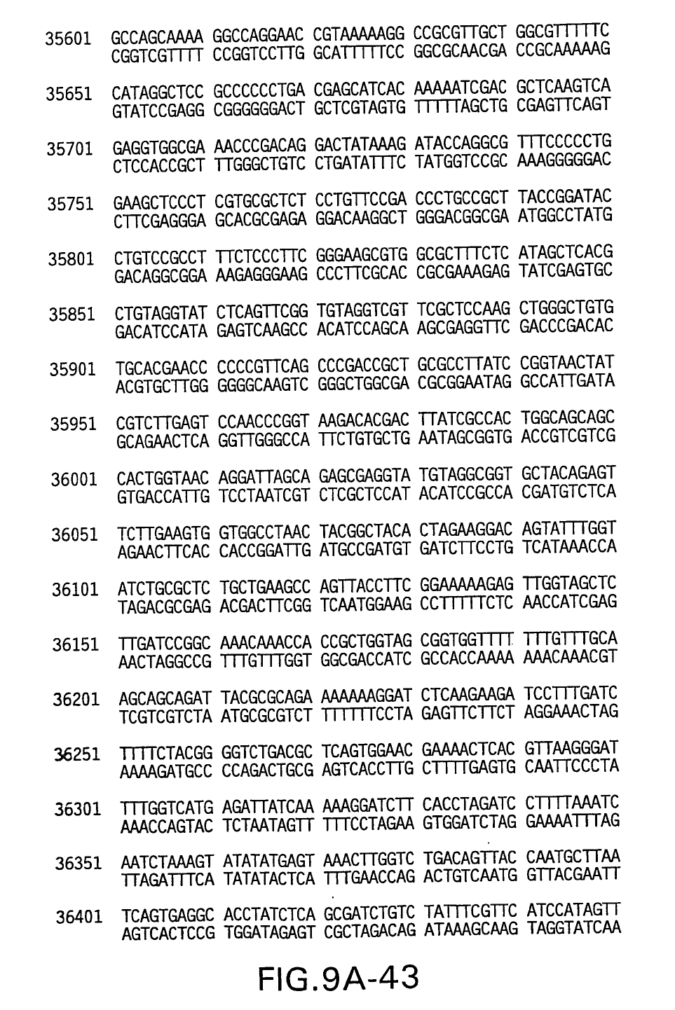

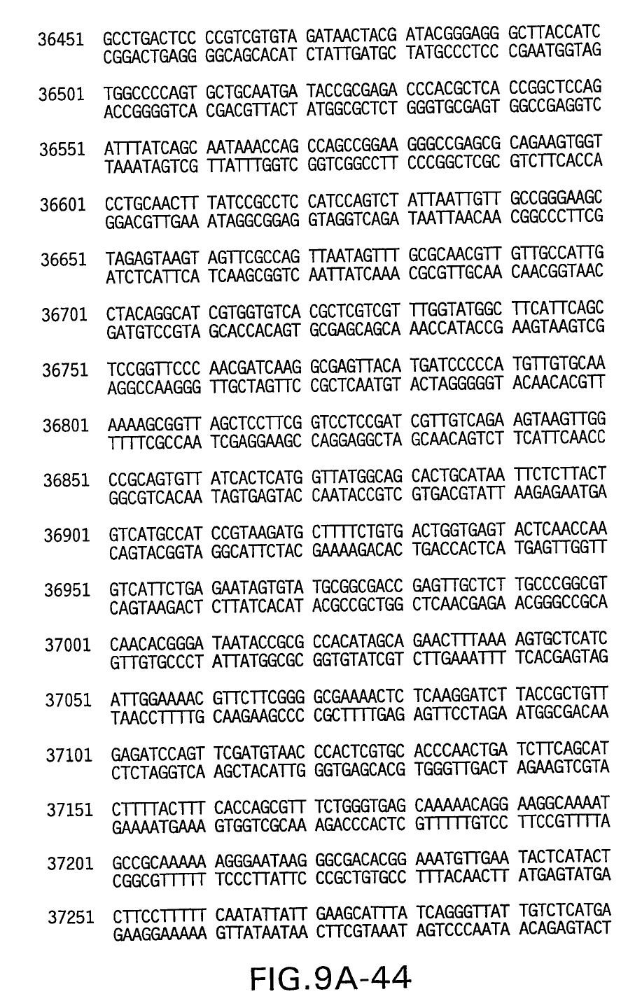

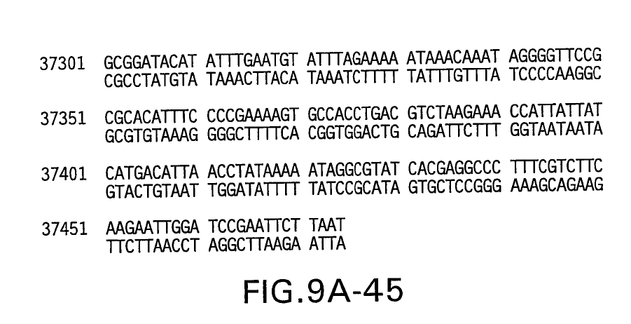

The general strategy used to recover pAd6E1-E3+as a bacterial plasmid is illustrated in FIG. 7. Cotransformation of BJ 5183 bacteria with purified wt Ad6 viral DNA and a second DNA fragment termed the Ad5 ITR cassette resulted in the circularization of the viral genome by homologous recombination. The ITR cassette contains sequences from the right (bp 33798 to 35935) and left (bp 1 to 341 and bp 3525 to 5767) end of the Ad5 genome separated by plasmid sequences containing a bacterial origin of replication and an ampicillin resistance gene. The ITR cassette contains a deletion of E1 sequences from Ad5 342 to 3524. The Ad5 sequences in the ITR cassette provide regions of homology with the purified Ad6 viral DNA in which recombination can occur.

Potential clones were screened by restriction analysis and one clone was selected as pAd6E1-E3+. This clone was then sequenced in it entirety. pAd6E1-E3+ contains Ad5 sequences from bp 1 to 341 and from bp 3525 to 5548, Ad6 bp 5542 to 33784, and Ad5 bp 33967 to 35935 (bp numbers refer to the wt sequence for both Ad5 and Ad6). pAd6E1-E3+contains the coding sequences for all Ad6 virion structural proteins which constitute its serotype specificity.

B. Construction of an Ad6 Pre-Adenovirus Plasmid Containing the HIV-1 gag gene

(1) Construction of Adenoviral Shuttle Vector:

The shuttle plasmid MRKpdelE1(Pac/pIX/pack450)+CMVminFL-gag-BGHpA was constructed by inserting a synthetic full-length codon-optimized HIV-1 gag gene into MRKpdelE1(Pac/pIX/pack450)+CMVmin+BGHpA(str.). MRKpdelE1(Pac/pIX/pack450)+CMVmin+BGHpA(str.) contains Ad5 sequences from bp 1 to 5792 with a deletion of E1 sequences from bp 451 to 3510. The HCMV promoter and BGH pA were inserted into the E1 deletion in an E1 parallel orientation with a unique BglII site separating them. The synthetic full-length codon-optimized HIV-1 gag gene was obtained from plasmid pV1Jns-HIV-FLgag-opt by BglII digestion, gel purified and ligated into the Bgl restriction endonuclease site in MRKpdelE1(Pac/pIX/pack450)+CMVmin+BGHpA(str.), generating plasmid MRKpdelE1(Pac/pIX/pack450)+CMVminFL-gag-BGHpA. The genetic structure of MRKpdelE1(Pac/pIX/pack450)+CMVminFL-gag-BGHpA was verified by PCR, restriction enzyme and DNA sequence analyses.

(2) Construction of Pre-Adenovirus Plasmid:

Shuttle plasmid MRKpdelE1(Pac/pIX/pack450)+CMVminFL-gag-BGHpA was digested with restriction enzymes Pac1 and Bst1 107I and then co-transformed into E. coli strain BJ5183 with linearized (ClaI-digested) adenoviral backbone plasmid, pAd6E1-E3+. The genetic structure of the resulting pMRKAd6gag was verified by restriction enzyme and DNA sequence analysis. The vectors were transformed into competent E. coli XL-1 Blue for large-scale production. The recovered plasmid was verified by restriction enzyme digestion and DNA sequence analysis, and by expression of the gag transgene in transient transfection cell culture.

pMRKAd6gag contains Ad5 bp 1 to 450 and from bp 3511 to 5548, Ad6 bp 5542 to 33784, and Ad5 bp 33967 to 35935 (bp numbers refer to the wt sequence for both Ad5 and Ad6). In the plasmid the viral ITRs are joined by plasmid sequences that contain the bacterial origin of replication and an ampicillin resistance gene.

C. Generation of Research-Grade Recombinant MRKAd6gag

To prepare virus for pre-clinical immunogenicity studies, the pre-adenovirus plasmid pMRKAd6gag was rescued as infectious virions in PER.C6® adherent monolayer cell culture. To rescue infectious virus, 10 μg of pMRKAd6gag was digested with restriction enzyme PacI (New England Biolabs) and transfected into a 6 cm dish of PER.C6® cells using the calcium phosphate co-precipitation technique (Cell Phect Transfection Kit, Amersham Pharmacia Biotech Inc.). PacI digestion releases the viral genome from plasmid sequences allowing viral replication to occur after entry into PER.C6®cells. Infected cells and media were harvested after complete viral cytopathic effect (CPE) was observed. The virus stock was amplified by multiple passages in PER.C6® cells. At the final passage virus was purified from the cell pellet by CsCl ultracentrifugation. The identity and purity of the purified virus was confirmed by restriction endonuclease analysis of purified viral DNA and by gag ELISA of culture supernatants from virus infected mammalian cells grown in vitro. For restriction analysis, digested viral DNA was end-labeled with P³³-dATP, size-fractionated by agarose gel electrophoresis, and visualized by autoradiography.

All viral constructs (adenovirus serotypes 5 and 6) were confirmed for Gag expression by Western blot analysis.

### EXAMPLE 4

### Immunization

Rhesus macaques were between 3-10 kg in weight. In all cases, the total dose of each vaccine was suspended in 1 mL of buffer. The macaques were anesthetized (ketamine/xylazine) and the vaccines were delivered intramuscularly (“i.m.”) in 0.5-mL aliquots into both deltoid muscles using tuberculin syringes (Becton-Dickinson, Franklin Lakes, N.J.). Peripheral blood mononuclear cells (PBMC) were prepared from blood samples collected at several time points during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

### EXAMPLE 5

### ELISPOT Assay

The IFN-γ ELISPOT assays for rhesus macaques were conducted following a previously described protocol (Allen et al., 2001 J. Virol. 75(2):738-749), with some modifications. For antigen-specific stimulation, a peptide pool was prepared from 20-amino acid (“aa”) peptides that encompass the entire HIV-1 gag sequence with 10-aa overlaps (Synpep Corp., Dublin, Calif.). To each well, 50 μL of 2-4×10⁵ peripheral blood mononuclear cells (PBMCs) were added. The cells were counted using Beckman Coulter Z2 particle analyzer with a lower size cut-off set at 80 femtoliters (“fL”). Either 50 μL of media or the gag peptide pool at 8 g/mL concentration per peptide were added to the PBMC. The samples were incubated at 37° C., 5% CO₂ for 20-24 hrs. Spots were developed accordingly and the plates were processed using custom-built imager and automatic counting subroutine based on the ImagePro platform (Silver Spring, Md.). The counts were normalized to 10⁶ cell input.

### EXAMPLE 6

### Anti-p24 ELISA

A modified competitive anti-p24 assay was developed using reagents from the Coulter p24 Antigen Assay kit (Beckman Coulter, Fullerton, Calif.). Briefly, to a 250-μL serum sample, 20 μL of Lyse Buffer and 15 μL of p24 antigen (9.375 pg) from the Coulter kit were added. After mixing, 200 μL of each sample were added to wells coated with a mouse anti-p24 mAb from the Coulter kit and incubated for 1.5 hr at 37° C. The wells were then washed and 200 μL of Biotin Reagent (polyclonal anti-p24-biotin) from the Coulter kit was added to each well. After a 1 hr, 37° C. incubation, detection was achieved using strepavidin-conjugated horseradish peroxidase and TMB substrate as described in the Coulter Kit. OD₄₅₀ₙₘ values were recorded. A 7-point standard curve was generated using a serial 2-fold dilution of serum from an HIV-seropositive individual. The lower cut-off for the assay is arbitrarily set at 10 milli Merck units/mL (mMU/mL) defined by a dilution of the seropositive human serum. This cutoff falls at approximately 65% of the maximum bound control signal which corresponds to that obtained with the diluent control only and with no positive analyte.

### EXAMPLE 7

### Intracellular Cytokine Staining

To 1 ml of 2×10⁶ PBMC/mL in complete RPMI media (in 17×100 mm round bottom polypropylene tubes (Sarstedt, Newton, N.C.)), anti-hCD28 (clone L293, Becton-Dickinson) and anti-hCD49d (clone L25, Becton-Dickinson) monoclonal antibodies were added to a final concentration of 1 μg/mL. For gag-specific stimulation, 10 μL of the peptide pool (at 0.4 mg/mL per peptide) were added. The tubes were incubated at 37° C. for 1 hr., after which 20 μL of 5 mg/mL of brefeldin A (Sigma) were added. The cells were incubated for 16 hours at 37° C., 5% CO₂, 90% humidity. 4 mL cold PBS/2% FBS were added to each tube and the cells were pelleted for 10 min at 1200 rpm. The cells were re-suspended in PBS/2% FBS and stained (30 min, 4° C.) for surface markers using several fluorescent-tagged mAbs: 20 μL per tube anti-hCD3-APC, clone FN-18 (Biosource); 20 μL anti-hCD8-PerCP, clone SK1 (Becton Dickinson); and 20 μL anti-hCD4-PE, clone SK3 (Becton Dickinson). Sample handling from this stage was conducted in the dark. The cells were washed and incubated in 750 μL 1×FACS Perm buffer (Becton Dickinson) for 10 minutes at room temperature. The cells were pelleted and re-suspended in PBS/2% FBS and 0.1 μg of FITC-anti-hIFN-γ, clone MD-1 (Biosource) was added. After 30 minutes of incubation, the cells were washed and re-suspended in PBS. Samples were analyzed using all four color channels of the Becton Dickinson FACS Calibur instrument. To analyze the data, the low side- and forward-scatter lymphocyte population was initially gated and a common fluorescence cut-off for cytokine-positive events was used for both CD4+ and CD8+ populations, and for both mock and gag-peptide reaction tubes of a sample.

### EXAMPLE 8

### Results

A. Immunization Regimen

Cohorts of 3-6 rhesus macaques were immunized following homologous and heterologous prime-boost regimens involving MRKAd5 and MRKAd6 vectors expressing the same codon-optimized HIV-1 gag. The immunization schedule is described in Table 1.

TABLE 1

| Group | Prime | Boost (month 6) |
|---|---|---|
| 1 | 10e9 vp MRKAd5-HIVgag | 10e9 vp MRKAd5-HIVgag |
| | at week 0, 4 | |
| 2 | 10e9 vp MRKAd6-HIVgag | 10e9 vp MRKAd6-HIVgag |
| | at week 0, 4 | |
| 3 | 10e9 vp MRKAd5-HIVgag | 10e9 pfu MRKAd6-HIVgag |
| | at week 0, 4 | |

B. T Cell Immune Responses

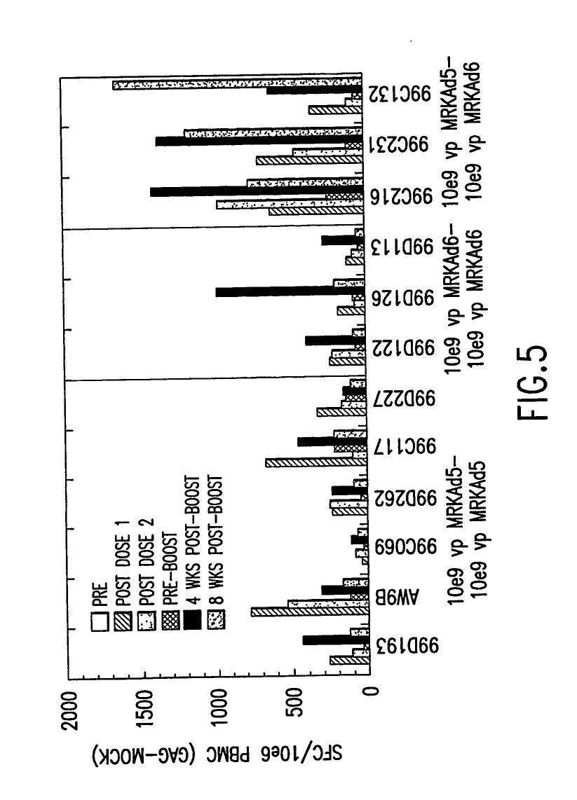

Vaccine-induced T cell responses against HIV-1 gag were quantified using IFN-gamma ELISPOT assay against a pool of 20-aa peptides that encompassed the entire protein sequence. The results are shown in FIG. 5. They are expressed as the number of spot-forming cells (SFC) per million peripheral blood mononuclear cells (PBMCs) that responded to the peptide pool minus the mock control.

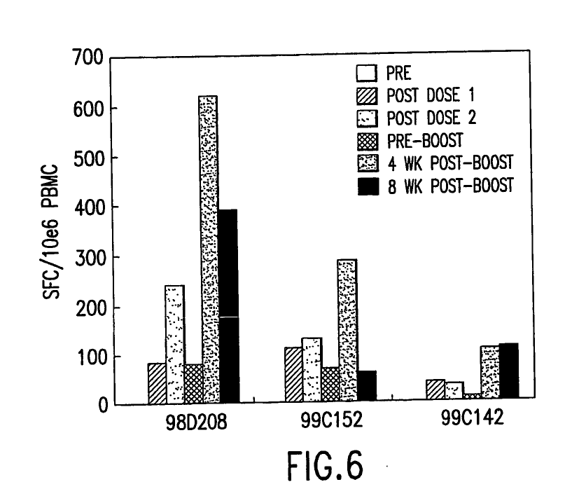

The Figure shows the T cell responses induced by two priming immunizations with 10e9 vp MRKAd5-HIVgag followed by a 10e9 vp MRKAd5-HIVgag booster after a long rest (a period of 20-23 weeks; 22 for the MRKAd6-MRKAd6 subjects; 22 for subjects 99D262, 99C117, and 99D227 of the MRKAd5-MRKAd5 group; and 23 for the remaining subjects). Administration of the same dose of MRKAd5 HIV-1 gag at approximately month 6 resulted in slight increases compared to the levels just prior to the boost; the post-boost levels were largely comparable to if not weaker than the peak levels before the boost. This is possibly due to the presence of neutralizing immunity generated against the vector by the first two immunizations. The responses after the boost did not surpass 500 gag-specific T cells per 10e6 PBMC, with a mean of 275 SFC/10e6 PBMC for all 6 monkeys. Similar results were observed when monkeys were given three of 10e9 vp MRKAd6 HIV-1 gag (at 0, 1, 6 months). In two out of the three monkeys, the post-boost levels did not surpass 500 SFC/10e6 PBMC. In contrast, when both modalities are combined in which animals were given two priming doses of 10e9 vp MRKAd5-HIVgag and a single booster shot of 10e9 pfu MRKAd6-HIVgag, the levels of gag-specific T cells increased to peak responses above 1000 SFC/10e6 PBMC for all 3 monkeys. The ability of MRKAd6-HIVgag to boost effectively MRKAd5-gag-primed immune responses more effectively is possibly due to the presence of neutralizing immunity generated against the MRKAd5 vector by the first two immunizations. The ability of Ad6 to boost primed responses was also evident using a lower priming dose of 107 vp of MRKAd5 HIV-1 gag (FIG. 6).

PBMCs from the vaccinees of the heterologous MRKAd5 prime-MRKAd6 boost regimen were analyzed for intracellular IFN-γ staining after the priming immunizations (wk 13) and after the booster immunizations (wk 31). The assay provided information on the relative amounts of CD4⁺ and CD8⁺ gag-specific T cells in the peripheral blood (Table 2). The results indicated that heterologous prime-boost immunization approach was able to elicit in rhesus macaques both mV-specific CD4+ and CD8+ T cells.

TABLE 2

| | | | Post Prime | | Post Boost | |
|---|---|---|---|---|---|---|
| Prime | Boost | ID | % CD4+ | % CD8+ | % CD4+ | % CD8+ |
| MRKAd5-HIVgag | MRKAd6-HIVgag | 99C216 | 0.05 | 0.21 | 0.10 | 1.45 |
| 10\hat{ }9 vp | 10\hat{ }9 pfu | 99C231 | 0.03 | 0.10 | 0.16 | 1.41 |
| wk 0, 4 | wk 27 | 99C132 | 0.00 | 0.02 | 0.04 | 0.15 |

Numbers reflect the percentages of circulating CD3+ lymphocytes that are either gag-specific CD4+ or gag-specific CD8+ cells. Mocks values have been subtracted.
*No detectable antigen-specific CD4+ T cells above background
**Collected at wk 35 instead of wk 31

C. Humoral Immune Responses

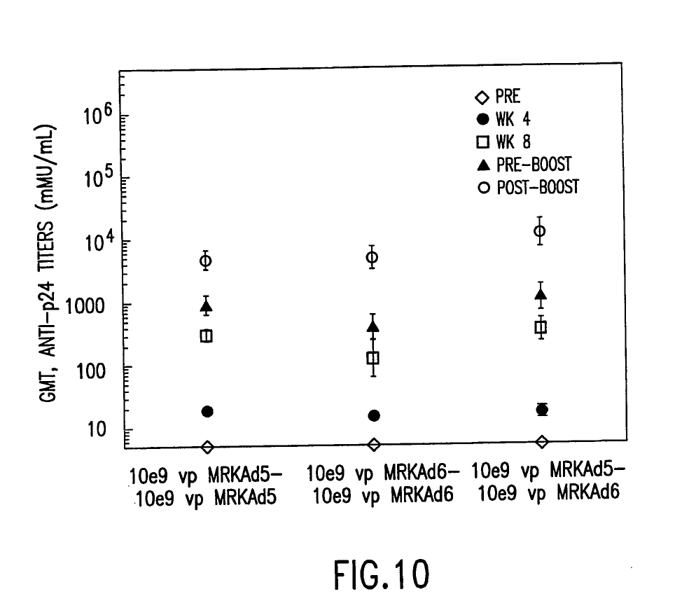

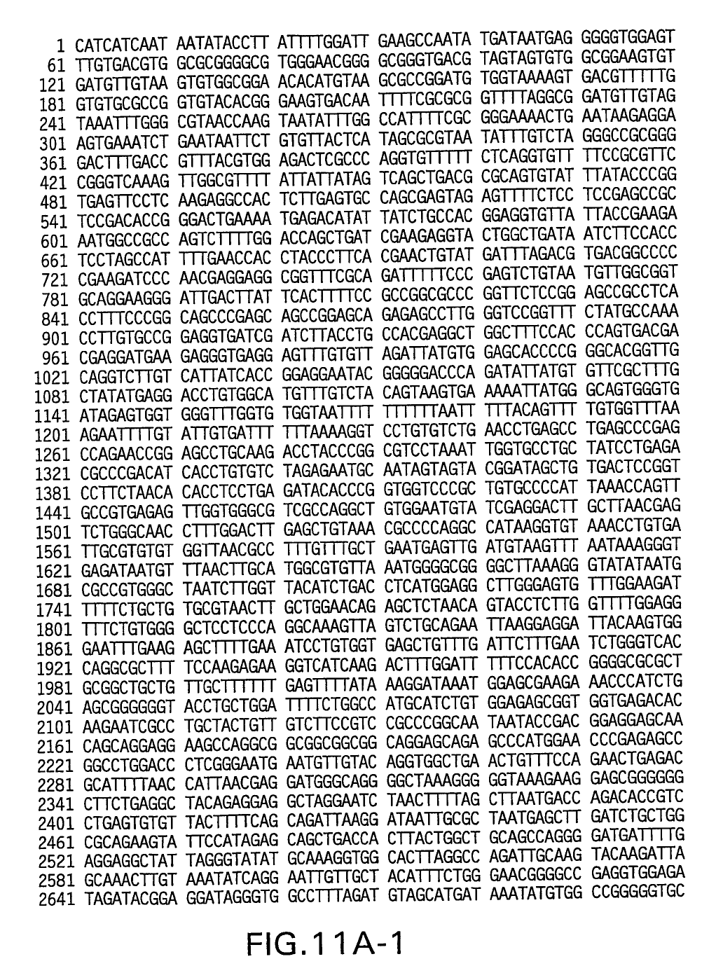

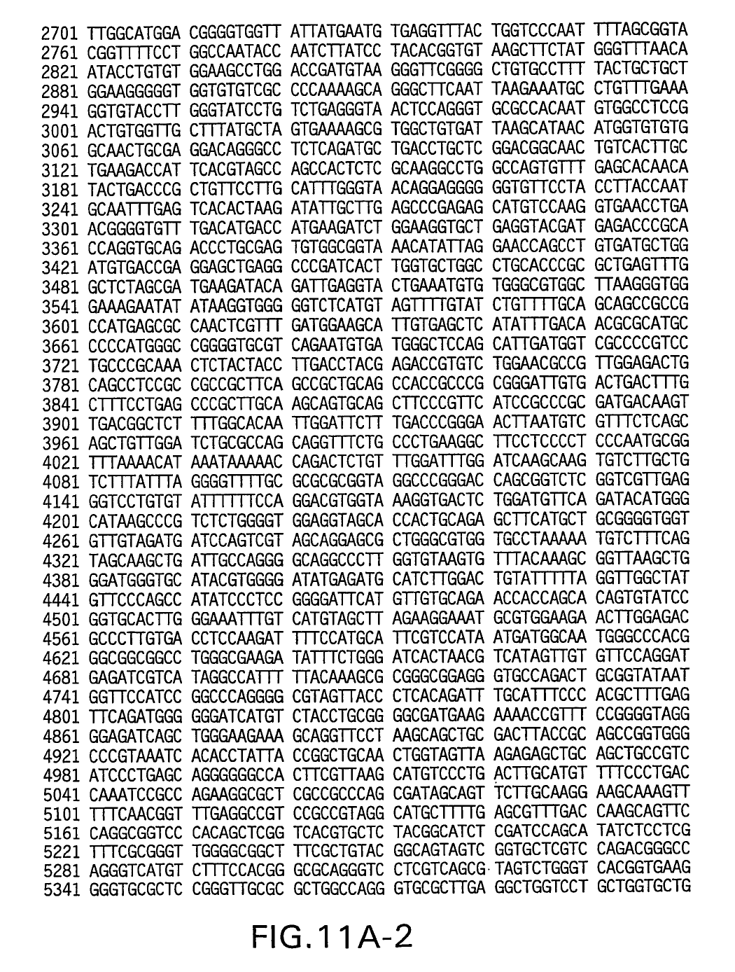

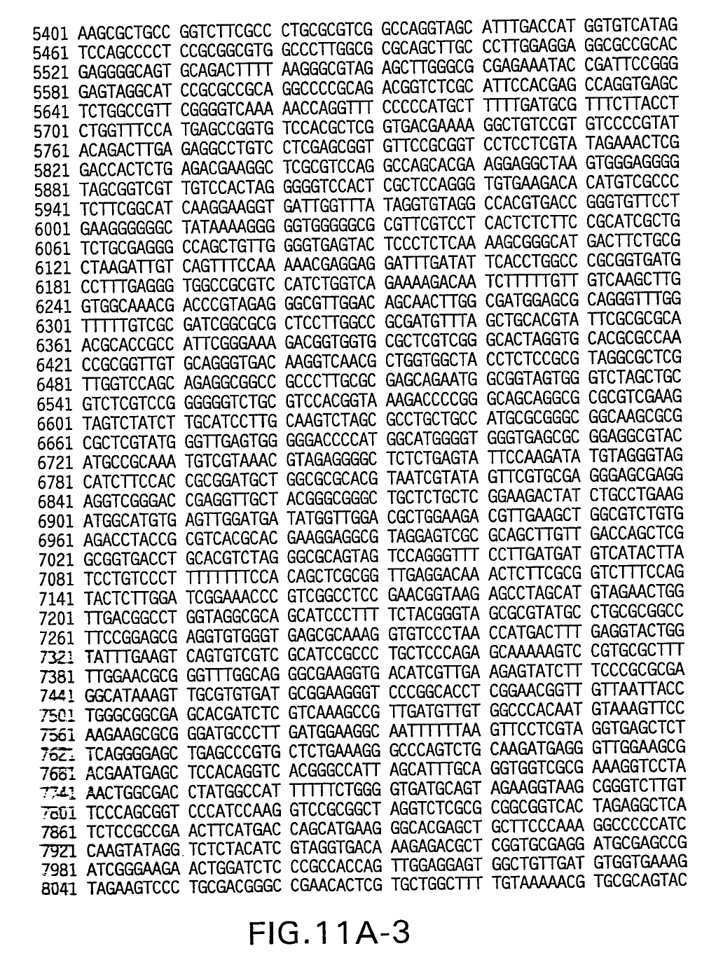

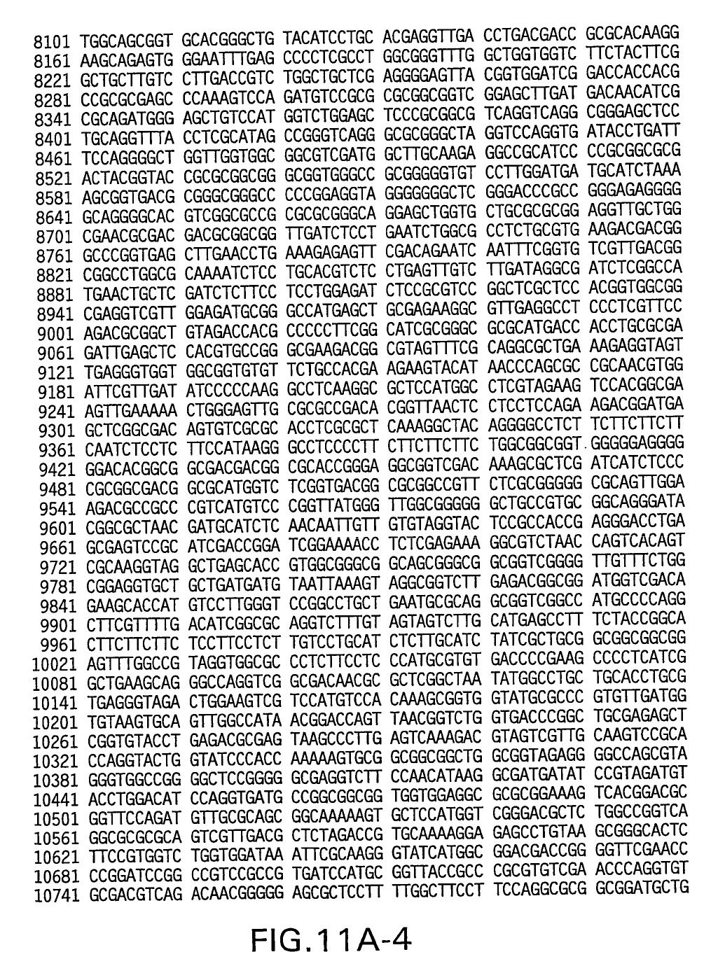

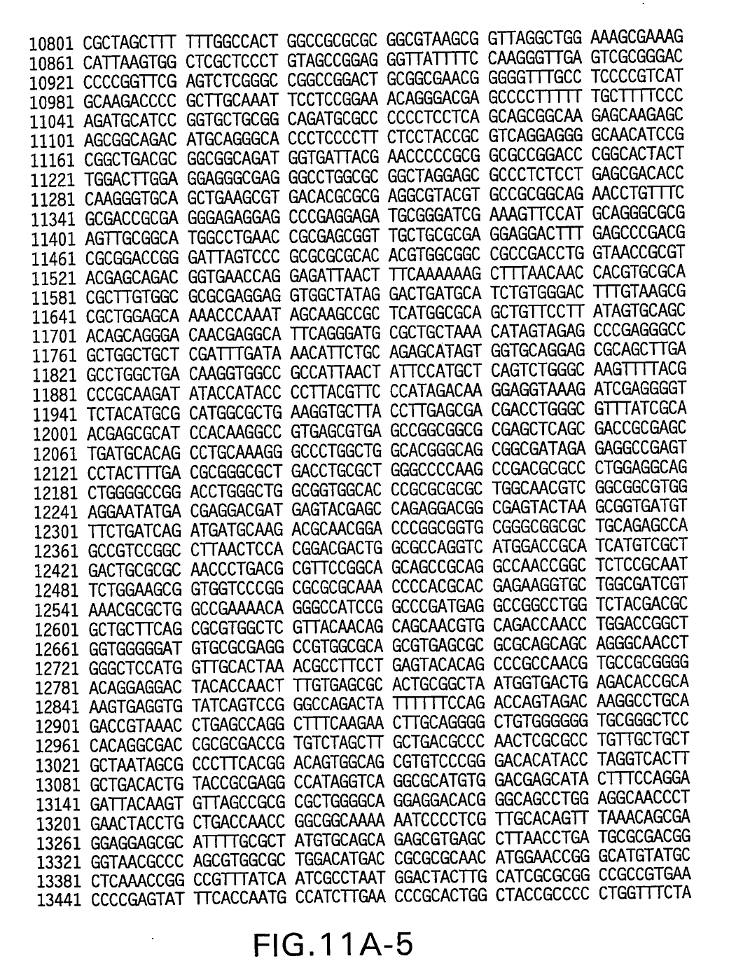

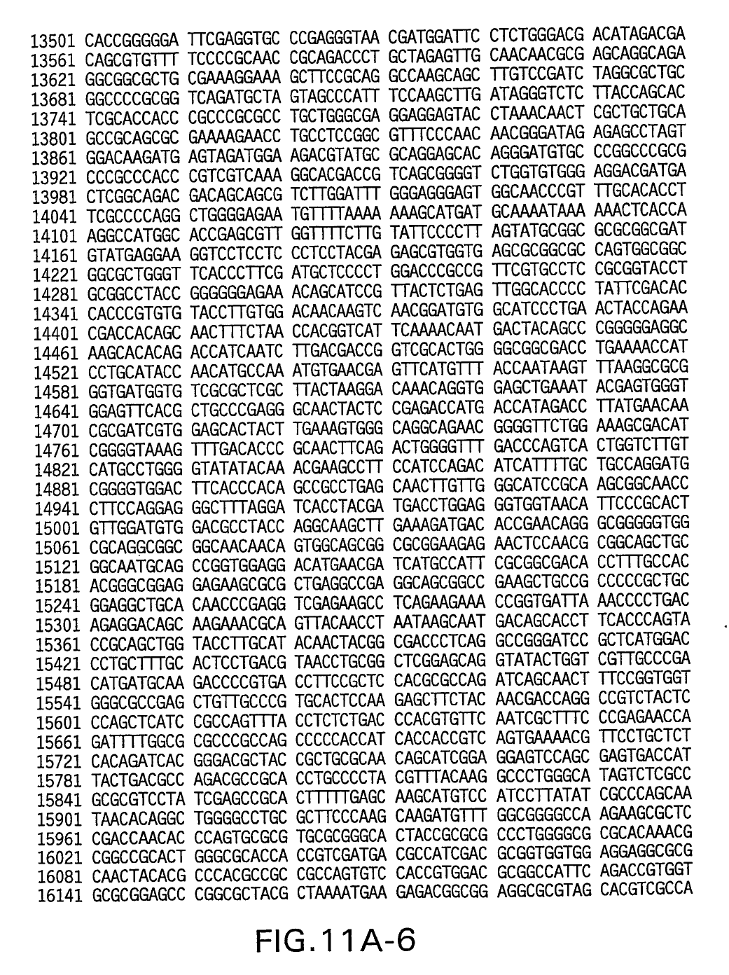

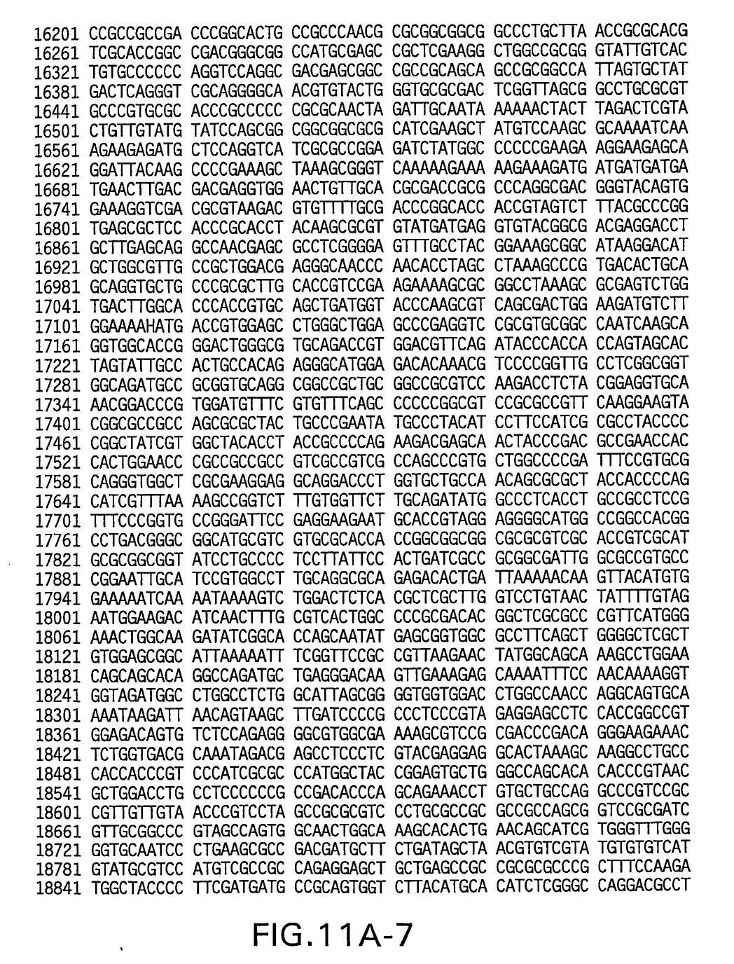

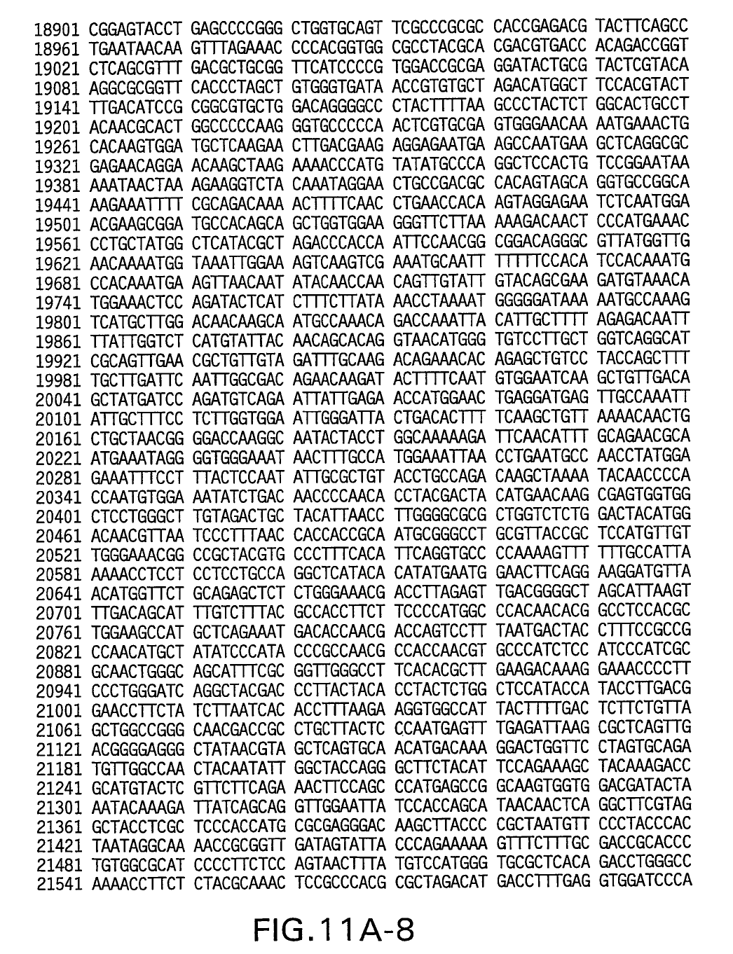

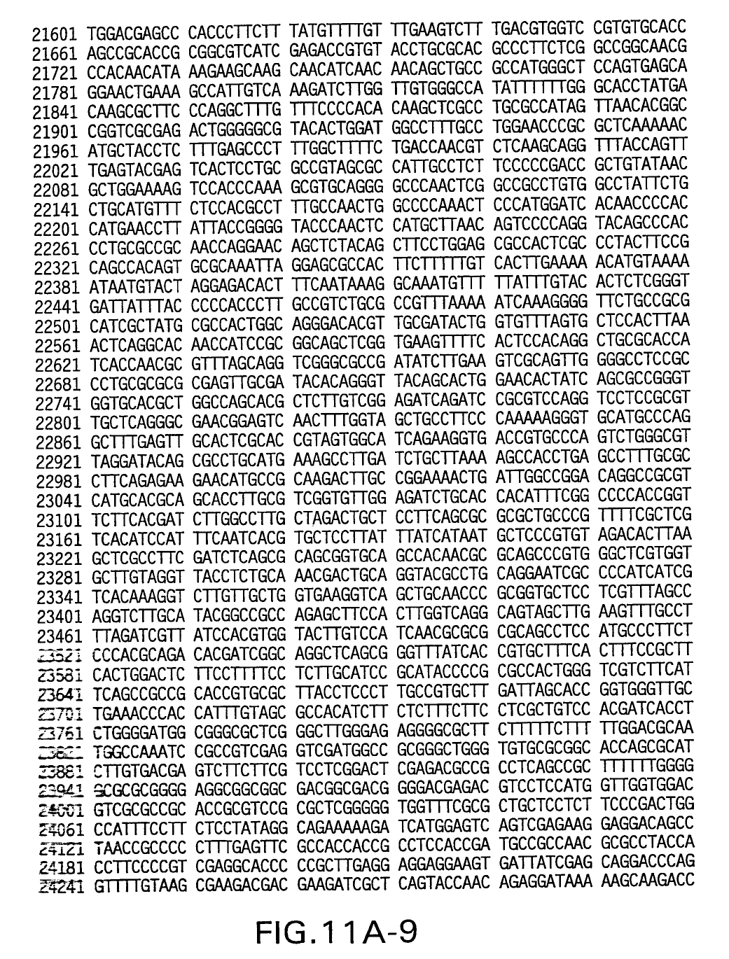

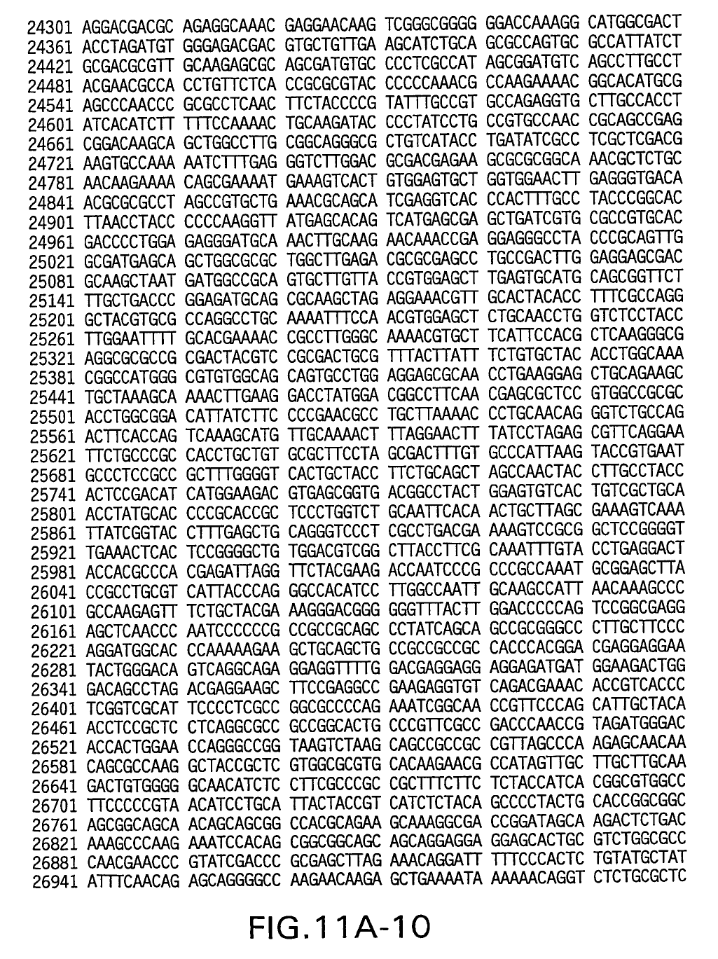

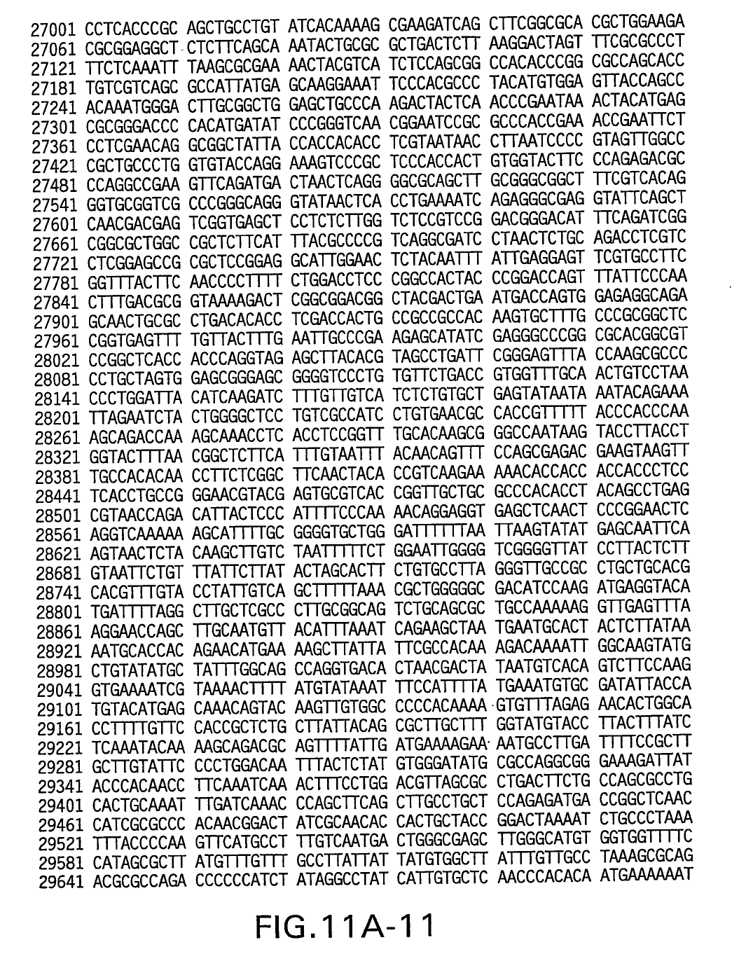

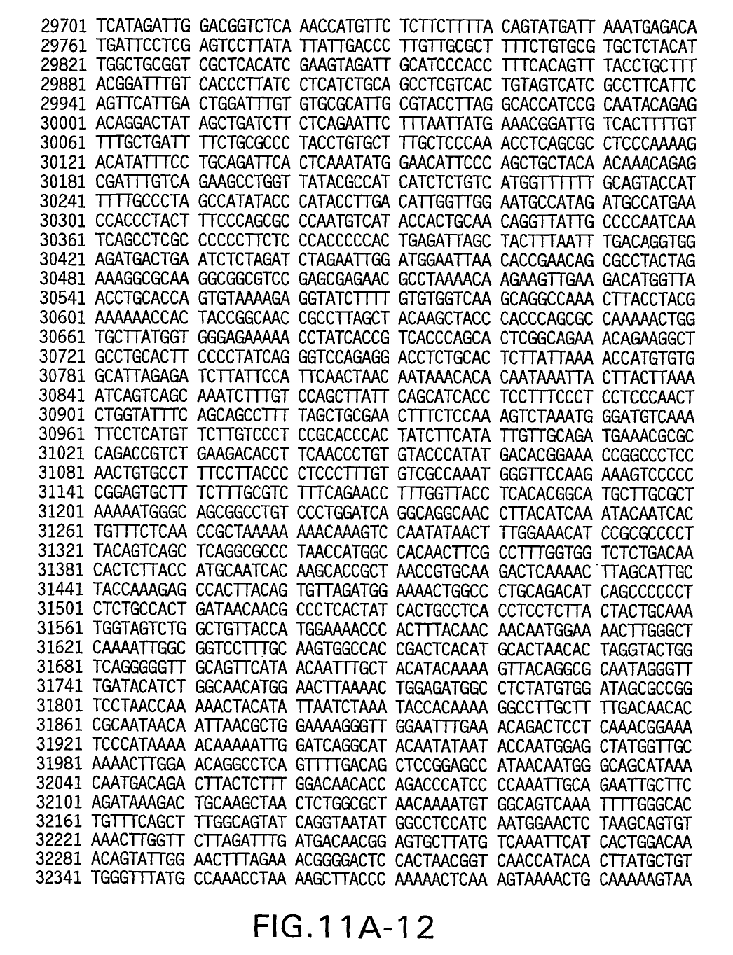

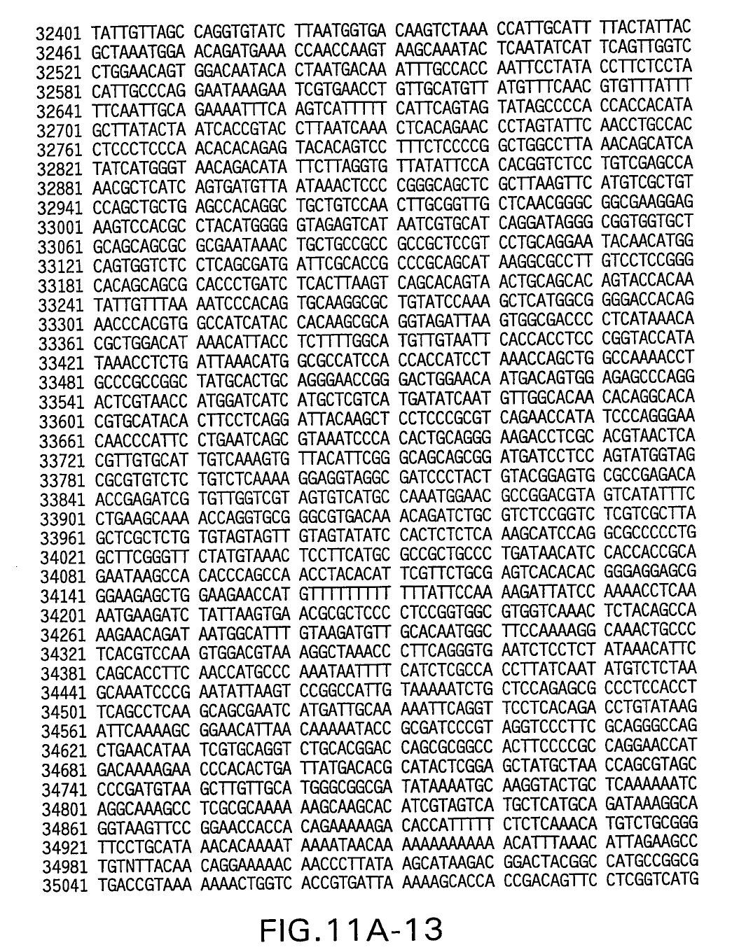

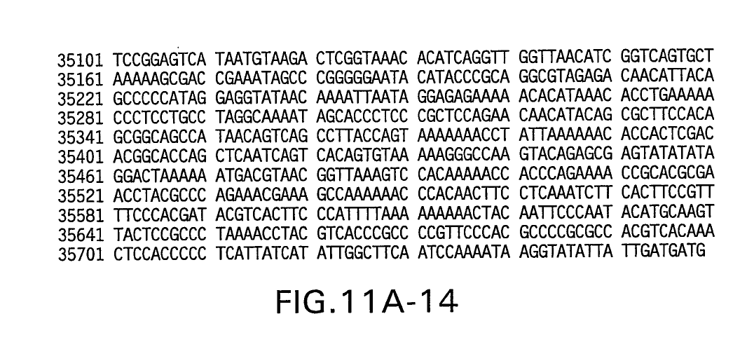

The p24-specific antibody titers were determined for each animal at several time points. The geometric mean titers for each cohort were calculated and shown in FIG. 10. Two doses of MRKAd5 HIV-1 gag or MRKAd6 HIV-1 gag were able to induce moderate levels of anti-p24 antibodies (about 1000 mMU/mL). Administration of the same viral vector booster resulted in 5-10 fold increase in the humoral immune responses. Boosting MRKAd5 HIV-1 gag primed monkeys with MRKAd6-gag resulted in a comparable in antibody levels. Boosting with the same virus can have its limitations, though, as the effect can be negatively impacted by any significant neutralizing Ad5-specific activity. The booster effect of a non-matched Ad serotype, by contrast, would not be affected by any pre-existing neutralizing titers directed at Ad5.

### EXAMPLE 9

### Generation of a Completely Adenoviral Serotype 6 Vector Construct

A. Construction of a Completely Ad6 Pre-Adenovirus Plasmid

An Ad6 based pre-adenovirus plasmid derived from Ad6 sequence and not constructed taking advantage of the homology between Ad5 and Ad6 can be generated and used to generate first generation Ad6 vectors. Homologous recombination is used to clone wtAd6 sequences into a bacterial plasmid.

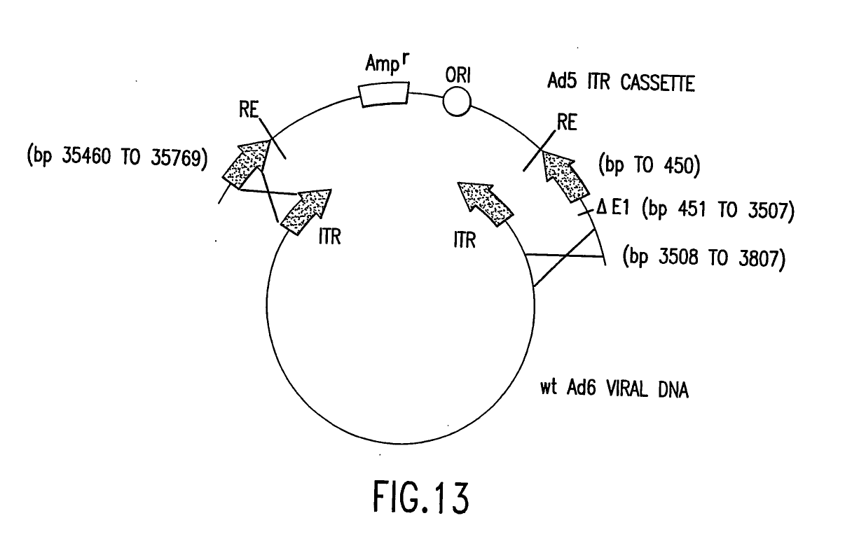

The general strategy used to recover such a pMRKAd6E1-bacterial plasmid is illustrated in FIG. 13. Basically, cotransformation of BJ 5183 bacteria with purified wt Ad6 viral DNA and a second DNA fragment termed the Ad6 ITR cassette would effectuate circularization of the viral genome by homologous recombination. The ITR cassette contains sequences from the right (bp 35460 to 35759) and left (bp 1 to 450 and bp 3508 to 3807) end of the Ad6 genome separated by plasmid sequences containing a bacterial origin of replication and an ampicillin resistance gene. These three segments were generated by PCR and cloned sequentially into pNEB193 (a commonly used commercially available cloning plasmid (New England Biolabs cat# N3051S) containing a bacterial origin of replication, ampicillin resistance gene and a multiple cloning site into which the PCR products are introduced), generating pNEBAd6-3 (the ITR cassette). The ITR cassette contains a deletion of E1 sequences from Ad5 451 to 3507. The Ad6 sequences in the ITR cassette provide regions of homology with the purified Ad6 viral DNA in which recombination can occur.

PMRKAd6E1—can then be used to generate first generation Ad6 vectors containing transgenes in E1 as described in the previous example.

### EXAMPLE 10

### In Vivo Immunogenicity

A. Immunization

Rhesus macaques were between 3-10 kg in weight. In all cases, the total dose of each vaccine was suspended in 1 mL of buffer. The macaques were anesthetized (ketamine/xylazine) and the vaccines were delivered i.m. in 0.5-mL aliquots into both deltoid muscles using tuberculin syringes (Becton-Dickinson, Franklin Lakes, N.J.). Peripheral blood mononuclear cells (PBMC) were prepared from blood samples collected at several time points during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. ELISPOT Assay

The IFN-γ ELISPOT assays for rhesus macaques were conducted following a previously described protocol (Allen et al., 2001 J. Virol. 75(2): 738-749), with some modifications. For antigen-specific stimulation, a peptide pool was prepared from 20-aa peptides that encompass the entire HIV-1 gag sequence with 10-aa overlaps (Synpep Corp., Dublin, Calif.). To each well, 50 μL of 2-4×10⁵ peripheral blood mononuclear cells (PBMCs) were added; the cells were counted using Beckman Coulter Z2 particle analyzer with a lower size cut-off set at 80 μL. Either 50 μL of media or the gag peptide pool at 8 μg/mL concentration per peptide were added to the PBMC. The samples were incubated at 37° C., 5% CO₂ for 20-24 hrs. Spots were developed accordingly and the plates were processed using custom-built imager and automatic counting subroutine based on the ImagePro platform (Silver Spring, Md.); the counts were normalized to 10⁶ cell input.

C. Results

Rare Serotype Vaccine Vector as a Heterologous Booster. A cohort of three rhesus macaques was immunized initially with 3 doses (wk 0, 4, 16) of 10⁸ vp of MRKAd5-gag. At wk 59, the animals received a booster vaccine of 1010 vp Ad35ΔE1gagΔE4Ad50Orf6 (an Ad35 virus engineered to contain an E1 deletion (from Ad35 bps 457-3402); and a deletion of E4 Orf6 (from Ad35 bps 31912-34418) substituted with Ad5 Orf6). A separate cohort of naïve animals received a single dose of the booster vaccine. The results of the IFN-γ ELISPOT analyses of PBMC collected during the course of the studies are shown in Table 3.

TABLE 3

| | | | Pre | | Prime^{b} | | Pre-Boost^{c} | | Post-Boost^{d} | |
|---|---|---|---|---|---|---|---|---|---|---|
| Animal | Prime (Wk 0, 4, 16) | Boost (Wk 59) | Mock^{a} | Gag^{a} | Mock | Gag | Mock | Gag | Mock | Gag |
| Monkey 11 | 10⁸ vp MRKAd5-gag | 10¹⁰ vp Ad35ΔE1gagΔE4Ad5Orf6 | 0 | 1 | 1 | 153 | 0 | 25 | 3 | 1120 |
| Monkey 12 | 10⁸ vp MRKAd5-gag | 10¹⁰ vp Ad35ΔE1gagΔE4Ad5Orf6 | 4 | 6 | 3 | 269 | 0 | 23 | 1 | 659 |
| Monkey 13 | 10⁸ vp MRKAd5-gag | 10¹⁰ vp Ad35ΔE1gagΔE4Ad5Orf6 | 1 | 3 | 3 | 150 | 0 | 10 | 1 | 489 |
| Monkey 14 | none | 10¹⁰ vp Ad35ΔE1gagΔE4Ad5Orf6 | 1 | 9 | ND^{e} | ND | ND | ND | 0 | 20 |
| Monkey 15 | none | 10¹⁰ vp Ad35ΔE1gagΔE4Ad5Orf6 | 3 | 3 | ND | ND | ND | ND | 1 | 81 |
| Monkey 16 | none | 10¹⁰ vp Ad35ΔE1gagΔE4Ad5Orf6 | 0 | 6 | ND | ND | ND | ND | 0 | 46 |

^{a}Mock, no peptide: gag, 20-mer peptide pool encompassing entire gag sequence
^{b}Peak response after 2 or 3 doses of the priming vaccine
^{c}Wk 59
^{d}4 wks after boost
^{e}ND, not determined

It is apparent that Ad35-based HIV vectors can be utilized to amplify the existing pools of HIV-specific T cells. The increases in the levels of gag-specific T cells from the pre-boost levels to those measured at 4 wks post boost were consistently larger than the levels induced by the same booster vaccine in naïve animals.

What is claimed is:

1. A method for inducing an enhanced immunological response against an HIV-1 antigen in a mammalian host, said method comprising the steps of:
(a) inoculating the mammalian host with a recombinant adenoviral vector of a first serotype which is at least partially deleted in E1 and devoid of E1 activity comprising a gene encoding an HIV-1 antigen or immunologically relevant modification thereof; and thereafter
(b) inoculating the mammalian host with a boosting immunization comprising a recombinant adenoviral vector of a second serotype which is at least partially deleted in E1 and devoid of E1 activity comprising a gene encoding the HIV-1 antigen or immunologically relevant modification thereof.

2. A method in accordance with claim 1 wherein the HIV-1 antigen is HIV-1 gag.

3. A method in accordance with claim 1 wherein the HIV-1 antigen is HIV-1 nef.

4. A method in acccordance with claim 1 wherein the HIV-1 antigen is HIV-1 pol.

5. A method in accordance with claim 1 wherein at least one gene encoding the HIV-1 antigen or immunologically relevant modification thereof comprises codons optimized for expression in a mammalian host.

6. A method in accordance with claim 1 wherein one or more of the recombinant adenoviral vectors comprise a gene expression cassette, said gene expression cassette which comprises:
(a) a nucleic acid encoding an HIV-1 antigen;
(b) a heterologous promoter operatively linked to the nucleic acid encoding the antigen; and
(c) a transcription termination sequence.

7. A method in accordance with claim 6 wherein the gene expression cassette in at least one of the recombinant adenoviral vectors is inserted into the E1 region.

8. A method in accordance with claim 6 wherein the promoter is an immediate early human cytomegalovirus promoter.

9. A method in accordance with claim 6 wherein the transcription termination sequence is a bovine growth hormone polyadenylation and transcription termination sequence.

10. A method for inducing an enhanced immunological response against an HIV-1 antigen in a mammalian host, said method comprising the steps of:
(a) inoculating the mammalian host with a recombinant adenoviral vector of serotype 5 at least partially deleted in E1 and devoid of E1 activity comprising a gene encoding an HIV-1 antigen or immunologically relevant modification thereof; and thereafter
(b) inoculating the mammalian host with a boosting immunization comprising a recombinant adenoviral vector of serotype 6 at least partially deleted in E1 and devoid of E1 activity comprising a gene encoding the HIV-1 antigen or immunologically relevant modification thereof.

11. A method in accordance with claim 10 wherein the recombinant adenoviral vector of step (a) is deleted of base pairs 451-3510.

12. A method in accordance with claim 10 wherein the recombinant adenoviral vector of step (b) is deleted of base pairs 451-3507.

13. A method in accordance with claim 10 wherein at least one gene encoding the HIV-1 antigen or immunologically relevant modification thereof comprises codons optimized for expression in a mammalian host.

14. A method in accordance with claim 10 wherein the HIV-1 antigen is HIV-1 gag.

15. A method in accordance with claim 10 wherein the HIV-1 antigen is HIV-1 nef.

16. A method in acccordance with claim 10 wherein the HIV-1 antigen is HIV-1 pol.

17. A method in accordance with claim 10 wherein one or more of the recombinant adenoviral vectors comprise a gene expression cassette, said gene expression cassette which comprises:
(a) a nucleic acid encoding an HIV-1 antigen;
(b) a heterologous promoter operatively linked to the nucleic acid encoding the antigen; and
(c) a transcription termination sequence.

18. A method in accordance with claim 17 wherein the gene expression cassette in at least one of the recombinant adenoviral vectors is inserted into the E1 region.

19. A method in accordance with claim 17 wherein the promoter is an immediate early human cytomegalovirus promoter.

20. A method in accordance with claim 17 wherein the transcription termination sequence is a bovine growth hormone polyadenylation and transcription termination sequence.

21. A method for inducing an enhanced immunological response against an HIV-1 gag antigen in a mammalian host, said method comprising the steps of:
(a) inoculating the mammalian host with a recombinant adenoviral vector of serotype 5 at least partially deleted in E1 and devoid of E1 activity comprising a gene encoding an HIV-1 gag antigen or immunologically relevant modification thereof; and thereafter
(b) inoculating the mammalian host with a boosting immunization comprising a recombinant adenoviral vector of serotype 6 at least partially deleted in E1 and devoid of E1 activity comprising a gene encoding the HIV-1 gag antigen or immunologically relevant modification thereof.

22. A method for inducing an enhanced immunological response against an HIV-1 antigen in a mammalian host, said method comprising the steps of:
(a) inoculating the mammalian host with a recombinant adenoviral vector of serotype 5 at least partially deleted in E1 and devoid of E1 activity comprising a gene encoding an HIV-1 antigen or immunologically relevant modification thereof; and thereafter
(b) inoculating the mammalian host with a boosting immunization comprising a recombinant adenoviral vector of serotype 35 at least partially deleted in E1 and devoid of E1 activity comprising a gene encoding the HIV-1 antigen or immunologically relevant modification thereof.

23. A method in accordance with claim 22 wherein at least one gene encoding the HIV-1 antigen or immunologically relevant modification thereof comprises codons optimized for expression in a mammalian host.

24. A method in accordance with claim 22 wherein the HIV-1 antigen is HIV-1 gag.

25. A method in accordance with claim 22 wherein the HIV-1 antigen is HIV-1 nef.

26. A method in acccordance with claim 22 wherein the HIV-1 antigen is HIV-1 pol.

27. A method in accordance with claim 22 wherein one or more of the recombinant adenoviral vectors comprise a gene expression cassette, said gene expression cassette which comprises:
(a) a nucleic acid encoding an HIV-1 antigen;
(b) a heterologous promoter operatively linked to the nucleic acid encoding the antigen; and
(c) a transcription termination sequence.

28. A method in accordance with claim 27 wherein the gene expression cassette in at least one of the recombinant adenoviral vectors is inserted into the E1 region.

29. A method in accordance with claim 27 wherein the promoter is an immediate early human cytomegalovirus promoter.

30. A method in accordance with claim 27 wherein the transcription termination sequence is a bovine growth hormone polyadenylation and transcription termination sequence.


SEQUENCE LISTING (5 sequences)

SEQ ID NO: 1 - DNA, 1521 bp, Artificial Sequence
  Codon optimized DNA encoding human HIV-1 gag
atgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagat

SEQ ID NO: 2 - DNA, 37474 bp, Artificial Sequence
  DNA encoding pMRKAd5 HIV-1 gag, coding
ttcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattataggcggccgcgatccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctaccatgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcggcgcgccgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaaacagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctcgtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctttaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaagaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcttaat

SEQ ID NO: 3 - DNA, 37474 bp, Artificial Sequence
  DNA encoding pMRKAd5 HIV-1 gag, noncoding
aagaattaattgtagtagttattatatggaataaaacctaacttcggttatactattactcccccacctcaaacactgcaccgcgccccgcacccttgccccgcccactgcatcatcacaccgccttcacactacaacgttcacaccgccttgtgtacattcgctgcctacaccgttttcactgcaaaaaccacacgcggccacatgtgtccttcactgttaaaagcgcgccaaaatccgcctacaacatcatttaaacccgcattggctcattctaaaccggtaaaagcgcccttttgacttattctccttcactttagacttattaaaacacaatgagtatcgcgcattataaacagatcccggcgcccctgaaactggcaaatgcacctctgagcgggtccacaaaaagagtccacaaaaggcgcaaggcccagtttcaaccgcaaaataataatatccgccggcgctaggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcgcctaaggggcacggttctcactctagatggtacccacgatcccgaagacacgacagaccaccactcgacctgttcaccctcttctagtccgactccggaccaccgttcttcttcatgttcgatttcgtgtaacacacccggaggtccctcgacctctccaaacgacacttgggaccggacgacctctggagactccccacgtccgtctaggacccggtcgaggtcgggagggacgtttgtccgagactcctcgactccagggacatgttgtgtcaccgatgggacatgacacacgtggtcttctaactacacttcctgtggttcctccgggacctcttctaactcctcctcgtcttgttcaggttcttcttccgggtcgtccgacgacgaccgtgtccgttgaggtcggtccacagggtcttgatggggtaacacgtcttggaggtcccggtctaccacgtggtccggtagaggggggcctgggacttacggacccacttccaccacctcctcttccggaagaggggactccactaggggtacaagagacgggacagactcccacggtggggggtcctggacttgtggtacgacttgtgtcaccccccggtagtccgacggtacgtctacgacttcctctggtagttactcctccgacgactcaccctgtccgacgtaggacacgtgcgaccggggtaacgggggccggtctactccctcgggtccccgagactgtaacgaccgtggtggaggtgggaggtcctcgtctaaccgacctactggttgttgggggggtagggacaccccctttagatgttctccacctagtaggacccggacttgttctaacactcctacatgagggggtggaggtaggacctgtagtccgtcccggggttcctcgggaagtccctgatacacctgtccaagatgttctgggactcccgactcgtccggagggtcctccacttcttgacctactgtctctgggacgaccacgtcttacggttgggactgacgttctggtaggacttccgggacccgggacgacggtgggacctcctctactactgtcggacggtcccccaccccccgggaccagtgttccggtcccacgaccgactccggtacagggtccactggttgaggcggtggtagtactacgtctccccgttgaagtccttggtctccttctgtcacttcacgaagttgacaccgttccacccggtgtaacggttcttgacatcccgggggtccttcttcccgacgaccttcacaccgttcctcccggtggtctacttcctgacgttactctccgtccggttgaaggacccgttttagaccgggagggtgttcccgtccggaccgttgaaggaggtcaggtccggactcgggtgtcggggagggctcctcaggaagtccaaacccctcctcttctggtgggggtcggtcttcgtcctcgggtaactgttcctcgacatgggggaccggagggactccagggacaaaccgttgctggggaggagggtcattttatttcgggcccgtctagacgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccggctagccgcgcggcatgactttacacacccgcaccgaattcccaccctttcttatatattccacccccagaatacatcaaaacatagacaaaacgtcgtcggcggcggcggtactcgtggttgagcaaactaccttcgtaacactcgagtataaactgttgcgcgtacgggggtacccggccccacgcagtcttacactacccgaggtcgtaactaccagcggggcaggacgggcgtttgagatgatggaactggatgctctggcacagaccttgcggcaacctctgacgtcggaggcggcggcgaagtcggcgacgtcggtggcgggcgccctaacactgactgaaacgaaaggactcgggcgaacgtttgtcacgtcgaagggcaagtaggcgggcgctactgttcaactgccgagaaaaccgtgttaacctaagaaactgggcccttgaattacagcaaagagtcgtcgacaacctagacgcggtcgtccaaagacgggacttccgaaggaggggagggttacgccaaattttgtatttattttttggtctgagacaaacctaaacctagttcgttcacagaacgacagaaataaatccccaaaacgcgcgcgccatccgggccctggtcgccagagccagcaactcccaggacacataaaaaaggtcctgcaccatttccactgagacctacaagtctatgtacccgtattcgggcagagaccccacctccatcgtggtgacgtctcgaagtacgacgccccaccacaacatctactaggtcagcatcgtcctcgcgacccgcaccacggatttttacagaaagtcatcgttcgactaacggtccccgtccgggaaccacattcacaaatgtttcgccaattcgaccctacccacgtatgcacccctatactctacgtagaacctgacataaaaatccaaccgatacaagggtcggtatagggaggcccctaagtacaacacgtcttggtggtcgtgtcacataggccacgtgaaccctttaaacagtacatcgaatcttcctttacgcaccttcttgaacctctgcgggaacactggaggttctaaaaggtacgtaagcaggtattactaccgttacccgggtgcccgccgccggacccgcttctataaagaccctagtgattgcagtatcaacacaaggtcctactctagcagtatccggtaaaaatgtttcgcgcccgcctcccacggtctgacgccatattaccaaggtaggccgggtccccgcatcaatgggagtgtctaaacgtaaagggtgcgaaactcaagtctacccccctagtacagatggacgccccgctacttcttttgccaaaggccccatcccctctagtcgacccttctttcgtccaaggactcgtcgacgctgaatggcgtcggccacccgggcatttagtgtggataatggccgacgttgaccatcaattctctcgacgtcgacggcagtagggactcgtccccccggtgaagcaattcgtacagggactgagcgtacaaaagggactggtttaggcggtcttccgcgagcggcgggtcgctatcgtcaagaacgttccttcgtttcaaaaagttgccaaactctggcaggcggcatccgtacgaaaactcgcaaactggttcgtcaaggtccgccagggtgtcgagccagtggacgagatgccgtagagctaggtcgtatagaggagcaaagcgcccaaccccgccgaaagcgacatgccgtcatcagccacgagcaggtctgcccggtcccagtacagaaaggtgcccgcgtcccaggagcagtcgcatcagacccagtgccacttccccacgcgaggcccgacgcgcgaccggtcccacgcgaactccgaccaggacgaccacgacttcgcgacggccagaagcgggacgcgcagccggtccatcgtaaactggtaccacagtatcaggtcggggaggcgccgcaccgggaaccgcgcgtcgaacgggaacctcctccgcggcgtgctccccgtcacgtctgaaaactcccgcatctcgaacccgcgctctttatggctaaggcccctcatccgtaggcgcggcgtccggggcgtctgccagagcgtaaggtgctcggtccactcgagaccggcaagccccagtttttggtccaaagggggtacgaaaaactacgcaaagaatggagaccaaaggtactcggccacaggtgcgagccactgcttttccgacaggcacaggggcatatgtctgaactctccggacaggagctcgccacaaggcgccaggaggagcatatctttgagcctggtgagactctgtttccgagcgcaggtccggtcgtgcttcctccgattcaccctccccatcgccagcaacaggtgatcccccaggtgagcgaggtcccacacttctgtgtacagcgggagaagccgtagttccttccactaaccaaacatccacatccggtgcactggcccacaaggacttccccccgatattttcccccacccccgcgcaagcaggagtgagagaaggcgtagcgacagacgctcccggtcgacaaccccactcatgagggagacttttcgcccgtactgaagacgcgattctaacagtcaaaggtttttgctcctcctaaactataagtggaccgggcgccactacggaaactcccaccggcgtaggtagaccagtcttttctgttagaaaaacaacagttcgaaccaccgtttgctgggcatctcccgcaacctgtcgttgaaccgctacctcgcgtcccaaaccaaaaacagcgctagccgcgcgaggaaccggcgctacaaatcgacgtgcataagcgcgcgttgcgtggcggtaagccctttctgccaccacgcgagcagcccgtggtccacgtgcgcggttggcgccaacacgtcccactgttccagttgcgaccaccgatggagaggcgcatccgcgagcaaccaggtcgtctccgccggcgggaacgcgctcgtcttaccgccatcccccagatcgacgcagagcaggccccccagacgcaggtgccatttctggggcccgtcgtccgcgcgcagcttcatcagatagaacgtaggaacgttcagatcgcggacgacggtacgcgcccgccgttcgcgcgcgagcatacccaactcaccccctggggtaccgtaccccacccactcgcgcctccgcatgtacggcgtttacagcatttgcatctccccgagagactcataaggttctatacatcccatcgtagaaggtggcgcctacgaccgcgcgtgcattagcatatcaagcacgctccctcgctcctccagccctggctccaacgatgcccgcccgacgagacgagccttctgatagacggacttctaccgtacactcaacctactataccaacctgcgaccttctgcaacttcgaccgcagacactctggatggcgcagtgcgtgcttcctccgcatcctcagcgcgtcgaacaactggtcgagccgccactggacgtgcagatcccgcgtcatcaggtcccaaaggaactactacagtatgaataggacagggaaaaaaaaggtgtcgagcgccaactcctgtttgagaagcgccagaaaggtcatgagaacctagcctttgggcagccggaggcttgccattctcggatcgtacatcttgaccaactgccggaccatccgcgtcgtagggaaaagatgcccatcgcgcatacggacgcgccggaaggcctcgctccacacccactcgcgtttccacagggactggtactgaaactccatgaccataaacttcagtcacagcagcgtaggcgggacgagggtctcgtttttcaggcacgcgaaaaaccttgcgcctaaaccgtcccgcttccactgtagcaacttctcatagaaagggcgcgctccgtatttcaacgcacactacgccttcccagggccgtggagccttgccaacaattaatggacccgccgctcgtgctagagcagtttcggcaactacaacaccgggtgttacatttcaaggttcttcgcgccctacgggaactaccttccgttaaaaaattcaaggagcatccactcgagaagtcccctcgactcgggcacgagactttcccgggtcagacgttctactcccaaccttcgctgcttactcgaggtgtccagtgcccggtaatcgtaaacgtccaccagcgctttccaggatttgaccgctggataccggtaaaaaagaccccactacgtcatcttccattcgcccagaacaagggtcgccagggtaggttccaagcgccgatccagagcgcgccgtcagtgatctccgagtagaggcggcttgaagtactggtcgtacttcccgtgctcgacgaagggtttccgggggtaggttcatatccagagatgtagcatccactgtttctctgcgagccacgctcctacgctcggctagcccttcttgacctagagggcggtggttaacctcctcaccgataactacaccactttcatcttcagggacgctgcccggcttgtgagcacgaccgaaaacatttttgcacgcgtcatgaccgtcgccacgtgcccgacatgtaggacgtgctccaactggactgctggcgcgtgttccttcgtctcacccttaaactcggggagcggaccgcccaaaccgaccaccagaagatgaagccgacgaacaggaactggcagaccgacgagctcccctcaatgccacctagcctggtggtgcggcgcgctcgggtttcaggtctacaggcgcgcgccgccagcctcgaactactgttgtagcgcgtctaccctcgacaggtaccagacctcgagggcgccgcagtccagtccgccctcgaggacgtccaaatggagcgtatctgcccagtcccgcgcccgatctaggtccactatggattaaaggtccccgaccaaccaccgccgcagctaccgaacgttctccggcgtaggggcgccgcgctgatgccatggcgcgccgcccgccacccggcgcccccacaggaacctactacgtagattttcgccactgcgcccgctcgggggcctccatcccccccgaggcctgggcggccctctcccccgtccccgtgcagccgcggcgcgcgcccgtcctcgaccacgacgcgcgcatccaacgaccgcttgcgctgctgcgccgccaactagaggacttagaccgcggagacgcacttctgctgcccgggccactcgaacttggactttctctcaagctgtcttagttaaagccacagcaactgccgccggaccgcgttttagaggacgtgcagaggactcaacagaactatccgctagagccggtacttgacgagctagagaaggaggacctctagaggcgcaggccgagcgaggtgccaccgccgctccagcaacctttacgcccggtactcgacgctcttccgcaactccggagggagcaaggtctgcgccgacatctggtgcgggggaagccgtagcgcccgcgcgtactggtggacgcgctctaactcgaggtgcacggcccgcttctgccgcatcaaagcgtccgcgactttctccatcaactcccaccaccgccacacaagacggtgcttcttcatgtattgggtcgcagcgttgcacctaagcaactatagggggttccggagttccgcgaggtaccggagcatcttcaggtgccgcttcaactttttgaccctcaacgcgcggctgtgccaattgaggaggaggtcttctgcctactcgagccgctgtcacagcgcgtggagcgcgagtttccgatgtccccggagaagaagaagaagttagaggagaaggtattcccggaggggaagaagaagaagaccgccgccaccccctcccccctgtgccgccgctgctgccgcgtggccctccgccagctgtttcgcgagctagtagaggggcgccgctgccgcgtaccagagccactgccgcgccggcaagagcgcccccgcgtcaaccttctgcggcgggcagtacagggccaatacccaaccgccccccgacggtacgccgtccctatgccgcgattgctacgtagagttgttaacaacacatccatgaggcggcggctccctggactcgctcaggcgtagctggcctagccttttggagagctctttccgcagattggtcagtgtcagcgttccatccgactcgtggcaccgcccgccgtcgcccgccgccagccccaacaaagaccgcctccacgacgactactacattaatttcatccgccagaactctgccgcctaccagctgtcttcgtggtacaggaacccaggccggacgacttacgcgtccgccagccggtacggggtccgaagcaaaactgtagccgcgtccagaaacatcatcagaacgtactcggaaagatggccgtgaagaagaagaggaaggagaacaggacgtagagaacgtagatagcgacgccgccgccgcctcaaaccggcatccaccgcgggagaaggagggtacgcacactggggcttcggggagtagccgacttcgtcccgatccagccgctgttgcgcgagccgattataccggacgacgtggacgcactcccatctgaccttcagtaggtacaggtgtttcgccaccatacgcgggcacaactaccacattcacgtcaaccggtattgcctggtcaattgccagaccactgggccgacgctctcgagccacatggactctgcgctcattcgggagctcagtttatgcatcagcaacgttcaggcgtggtccatgaccatagggtggtttttcacgccgccgccgaccgccatctccccggtcgcatcccaccggccccgaggcccccgctctagaaggttgtattccgctactataggcatctacatggacctgtaggtccactacggccgccgccaccacctccgcgcgcctttcagcgcctgcgccaaggtctacaacgcgtcgccgtttttcacgaggtaccagccctgcgagaccggccagtccgcgcgcgttagcaactgcgagatctggcacgttttcctctcggacattcgcccgtgagaaggcaccagaccacctatttaagcgttcccatagtaccgcctgctggccccaagctcggggcataggccggcaggcggcactaggtacgccaatggcgggcgcacagcttgggtccacacgctgcagtctgttgccccctcacgaggaaaaccgaaggaaggtccgcgccgccgacgacgcgatcgaaaaaaccggtgaccggcgcgcgtcgcattcgccaatccgacctttcgctttcgtaattcaccgagcgagggacatcggcctcccaataaaaggttcccaactcagcgccctgggggccaagctcagagcctggccggcctgacgccgcttgcccccaaacggaggggcagtacgttctggggcgaacgtttaaggaggcctttgtccctgctcggggaaaaaacgaaaagggtctacgtaggccacgacgccgtctacgcggggggaggagtcgtcgccgttctcgttctcgtcgccgtctgtacgtcccgtgggaggggaggaggatggcgcagtcctccccgctgtaggcgccaactgcgccgtcgtctaccactaatgcttgggggcgccgcggcccgggccgtgatggacctgaacctcctcccgctcccggaccgcgccgatcctcgcgggagaggactcgccgtgggttcccacgtcgacttcgcactatgcgcactccgcatgcacggcgccgtcttggacaaagcgctggcgctccctctcctcgggctcctctacgccctagctttcaaggtgcgtcccgcgctcgacgccgtaccggacttagcgctcgccaacgacgcgctcctcctgaaactcgggctgcgcgcttggccctaatcagggcgcgcgcgtgtgcaccgccggcggctggaccattggcgtatgctcgtctgccacttggtcctctaattgaaagttttttcgaaattgttggtgcacgcatgcgaacaccgcgcgctcctccaccgatatcctgactacgtagacaccctgaaacattcgcgcgacctcgttttgggtttatcgttcggcgagtaccgcgtcgacaaggaatatcacgtcgtgtcgtccctgttgctccgtaagtccctacgcgacgatttgtatcatctcgggctcccggcgaccgacgagctaaactatttgtaggacgtctcgtatcaccacgtcctcgcgtcgaactcggaccgactgttccaccggcggtagttgataaggtacgaatcggacccgttcaaaatgcgggcgttctatatggtatggggaatgcaagggtatctgttcctccatttctagctccccaagatgtacgcgtaccgcgacttccacgaatggaactcgctgctggacccgcaaatagcgttgctcgcgtaggtgttccggcactcgcactcggccgccgcgctcgagtcgctggcgctcgactacgtgtcggacgtttcccgggaccgaccgtgcccgtcgccgctatctctccggctcaggatgaaactgcgcccgcgactggacgcgacccggggttcggctgcgcgggacctccgtcgaccccggcctggacccgaccgccaccgtgggcgcgcgcgaccgttgcagccgccgcacctccttatactgctcctgctactcatgctcggtctcctgccgctcatgattcgccactacaaagactagtctactacgttctgcgttgcctgggccgccacgcccgccgcgacgtctcggtcggcaggccggaattgaggtgcctgctgaccgcggtccagtacctggcgtagtacagcgactgacgcgcgttaggactgcgcaaggccgtcgtcggcgtccggttggccgagaggcgttaagaccttcgccaccagggccgcgcgcgtttggggtgcgtgctcttccacgaccgctagcatttgcgcgaccggcttttgtcccggtaggccgggctgctccggccggaccagatgctgcgcgacgaagtcgcgcaccgagcaatgttgtcgccgttgcacgtctggttggacctggccgaccaccccctacacgcgctccggcaccgcgtcgcactcgcgcgcgtcgtcgtcccgttggacccgaggtaccaacgtgatttgcggaaggactcatgtgtcgggcggttgcacggcgcccctgtcctcctgatgtggttgaaacactcgcgtgacgccgattaccactgactctgtggcgtttcactccacatggtcagacccggtctgataaaaaaggtctggtcatctgttccggacgtctggcatttggactcggtccgaaagtttttgaacgtccccgacaccccccacgcccgagggtgtccgctggcgcgctggcacagatcgaacgactgcgggttgagcgcggacaacgacgacgattatcgcgggaagtgcctgtcaccgtcgcacagggccctgtgtatggatccagtgaacgactgtgacatggcgctccggtatccagtccgcgtacacctgctcgtatgaaaggtcctctaatgttcacagtcggcgcgcgaccccgtcctcctgtgcccgtcggacctccgttgggatttgatggacgactggttggccgccgtcttctaggggagcaacgtgtcaaatttgtcgctcctcctcgcgtaaaacgcgatgcacgtcgtctcgcactcggaattggactacgcgctgccccattgcgggtcgcaccgcgacctgtactggcgcgcgttgtaccttggcccgtacatacggagtttggccggcaaatagttggcggattacctgatgaacgtagcgcgccggcggcacttggggctcataaagtggttacggtagaacttgggcgtgaccgatggcgggggaccaaagatgtggccccctaagctccacgggctcccattgctacctaaggagaccctgctgtatctgctgtcgcacaaaaggggcgttggcgtctgggacgatctcaacgttgtcgcgctcgtccgtctccgccgcgacgctttcctttcgaaggcgtccggttcgtcgaacaggctagatccgcgacgccggggcgccagtctacgatcatcgggtaaaggttcgaactatcccagagaatggtcgtgagcgtggtgggcgggcgcggacgacccgctcctcctcatggatttgttgagcgacgacgtcggcgtcgcgctttttttggacggaggccgtaaagggttgttgccctatctctcggatcacctgttctactcatctaccttctgcatgcgcgtcctcgtgtccctgcacggtccgggcgcgggcgggtgggcagcagtttccgtgctggcagtcgccccagaccacaccctcctgctactgagccgtctgctgtcgtcgcaggacctaaaccctccctcaccgttgggcaaacgcgtggaagcggggtccgacccctcttacaaaatttttttttttttcgtactacgttttattttttgagtggttccggtaccgtggctcgcaaccaaaagaacataaggggaatcatacgccgcgcgccgctacatactccttccaggaggagggaggatgctctcacaccactcgcgccgcggtcaccgccgccgcgacccaagagggaagctacgaggggacctgggcggcaaacacggaggcgccatggacgccggatggcccccctctttgtcgtaggcaatgagactcaaccgtggggataagctgtggtgggcacacatggaccacctgttgttcagttgcctacaccgtagggacttgatggtcttgctggtgtcgttgaaagactggtgccagtaagttttgttactgatgtcgggccccctccgttcgtgtgtctggtagttagaactgctggccagcgtgaccccgccgctggacttttggtaggacgtatggttgtacggtttacacttgctcaagtacaaatggttattcaaattccgcgcccactaccacagcgcgaacggatgattcctgttagtccacctcgactttatgctcacccacctcaagtgcgacgggctcccgttgatgaggctctggtactggtatctggaatacttgttgcgctagcacctcgtgatgaactttcacccgtctgtcttgccccaagacctttcgctgtagccccatttcaaactgtgggcgttgaagtctgaccccaaactggggcagtgaccagaacagtacggaccccatatatgtttgcttcggaaggtaggtctgtagtaaaacgacggtcctacgccccacctgaagtgggtgtcggcggactcgttgaacaacccgtaggcgttcgccgttgggaaggtcctcccgaaatcctagtggatgctactagacctcccaccattgtaagggcgtgacaacctacacctgcggatggtccgctcgaactttctactgtggcttgtcccgcccccaccgcgtccgccgtcgttgtcgtcaccgtcgccgcgccttctcttgaggttgcgccgtcggcgccgttacgtcggccacctcctgtacttgctagtacggtaagcgccgctgtggaaacggtgtgcccgactcctcttcgcgcgactccggcttcgtcgccggcttcgacggcgggggcgacgcgttgggctccagctcttcggagtcttctttggccactagtttggggactgtctcctgtcgttctttgcgtcaatgttggattattcgttactgtcgtggaagtgggtcatggcgtcgaccatggaacgtatgttgatgccgctgggagtctggccttaggcgagtacctgggacgaaacgtgaggactgcattggacgccgagcctcgtccagatgaccagcaacggtctgtactacgttctggggcactggaaggcgaggtgcgcggtctagtcgttgaaaggccaccacccgcggctcgacaacgggcacgtgaggttctcgaagatgttgctggtccggcagatgagggttgagtaggcggtcaaatggagagactgggtgcacaagttagcgaaagggctcttggtctaaaaccgcgcgggcggtcgggggtggtagtggtggcagtcacttttgcaaggacgagagtgtctagtgccctgcgatggcgacgcgttgtcgtagcctcctcaggtcgctcactggtaatgactgcggtctgcggcgtggacggggatgcaaatgttccgggacccgtatcagagcggcgcgcaggatagctcggcgtgaaaaactcgttcgtacaggtaggaatatagcgggtcgttattgtgtccgaccccggacgcgaagggttcgttctacaaaccgccccggttcttcgcgaggctggttgtgggtcacgcgcacgcgcccgtgatggcgcgcgggaccccgcgcgtgtttgcgccggcgtgacccgcgtggtggcagctactgcggtagctgcgccaccacctcctccgcgcgttgatgtgcgggtgcggcggtggtcacaggtgtcacctgcgccggtaagtctggcaccacgcgcctcgggccgcgatacgattttacttctctgccgcctccgcgcatcgtgcagcggtggcggcggctgggccgtgacggcgggttgcgcgccgccgccgggacgaattggcgcgtgcagcgtggccggctgcccgccggtacgcccggcgagcttccgaccggcgcccataacagtgacacggggggtccaggtccgctgctcgccggcggcgtcgtcggcgccggtaatcacgatactgagtcccagcgtccccgttgcacataacccacgcgctgagccaatcgccggacgcgcacgggcacgcgtgggcggggggcgcgttgatctaacgttcttttttgatgaatctgagcatgacaacatacataggtcgccgccgccgcgcgttgcttcgatacaggttcgcgttttagtttcttctctacgaggtccagtagcgcggcctctagataccggggggcttcttccttctcgtcctaatgttcggggctttcgatttcgcccagtttttctttttctttctactactactacttgaactgctgctccaccttgacgacgtgcgatggcgcgggtccgctgcccatgtcacctttccagctgcgcattttgcacaaaacgctgggccgtggtggcatcagaaatgcgggccactcgcgaggtgggcgtggatgttcgcgcacatactactccacatgccgctgctcctggacgaactcgtccggttgctcgcggagcccctcaaacggatgcctttcgccgtattcctgtacgaccgcaacggcgacctgctcccgttgggttgtggatcggatttcgggcattgtgacgtcgtccacgacgggcgcgaacgtggcaggcttcttttcgcgccggatttcgcgctcagaccactgaaccgtgggtggcacgtcgactaccatgggttcgcggtcgctgaccttctacagaaccttttttactggcaccttggacccgacctcgggctccaggcgcacgccggttagttcgtccaccgcggccctgacccgcacgtctggcacctgcaagtctatgggtgatggtcatcgtggtcataacggtggcggtgtctcccgtacctctgtgtttgcaggggccaacggagtcgccaccgcctacggcgccacgtccgccagcgacgccggcgcaggttctggagatgcctccacgtttgcctgggcacctacaaagcgcaaagtcggggggccgcgggcgcggcaagctccttcatgccgcggcggtcgcgcgatgacgggcttatacgggatgtaggaaggtaacgcggatgggggccgatagcaccgatgtggatggcggggtcttctgctcgttgatgggctgcggcttggtggtgaccttgggcggcggcggcagcggcagcggtcgggcacgaccggggctaaaggcacgcgtcccaccgagcgcttcctccgtcctgggaccacgacggttgtcgcgcgatggtggggtcgtagcaaattttcggccagaaacaccaagaacgtctataccgggagtggacggcggaggcaaagggccacggccctaaggctccttcttacgtggcatcctccccgtaccggccggtgccggactgcccgccgtacgcagcacgcgtggtggccgccgccgcgcgcagcgtggcagcgtacgcgccgccataggacggggaggaataaggtgactagcggcgccgctaaccgcggcacgggccttaacgtaggcaccggaacgtccgcgtctctgtgactaatttttgttcaacgtacacctttttagttttatttttcagacctgagagtgcgagcgaaccaggacattgataaaacatcttaccttctgtagttgaaacgcagagaccggggcgctgtgccgagcgcgggcaagtaccctttgaccgttctatagccgtggtcgttatactcgccaccgcggaagtcgaccccgagcgacacctcgccgtaatttttaaagccaaggtggcaattcttgataccgtcgttccggaccttgtcgtcgtgtccggtctacgactccctattcaactttctcgttttaaaggttgttttccaccatctaccggaccggagaccgtaatcgccccaccacctggaccggttggtccgtcacgttttattctaattgtcattcgaactaggggcgggagggcatctcctcggaggtggccggcacctctgtcacagaggtctccccgcaccgcttttcgcaggcgcggggctgtcccttctttgagaccactgcgtttatctgctcggagggagcatgctcctccgtgatttcgttccggacgggtggtgggcagggtagcgcgggtaccgatggcctcacgacccggtcgtgtgtgggcattgcgacctggacggaggggggcggctgtgggtcgtctttggacacgacggtccgggctggcggcaacaacattgggcaggatcggcgcgcagggacgcggcgcggcggtcgccaggcgctagcaacgccgggcatcggtcaccgttgaccgtttcgtgtgacttgtcgtagcacccagacccccacgttagggacttcgcggctgctacgaagactatcgattgcacagcatacacacagtacatacgcaggtacagcggcggtctcctcgacgactcggcggcgcgcgggcgaaaggttctaccgatggggaagctactacggcgtcaccagaatgtacgtgtagagcccggtcctgcggagcctcatggactcggggcccgaccacgtcaaacgggcgcggtggctctgcatgaagtcggacttattgttcaaatctttggggtgccaccgcggatgcgtgctgcactggtgtctggccagggtcgcaaactgcgacgccaagtagggacacctggcactcctatgacgcatgagcatgttccgcgccaagtgggatcgacacccactattggcacacgacctgtaccgaaggtgcatgaaactgtaggcgccgcacgacctgtccccgggatgaaaattcgggatgagaccgtgacggatgttgcgggaccgagggttcccacggggtttaggaacgcttaccctacttcgacgatgacgagaactttatttggatcttcttctcctgctactgttgcttctgcttcatctgctcgttcgactcgtcgttttttgagtgcataaacccgtccgcggaataagaccatatttataatgtttcctcccataagtttatccacagcttccagtttgtggatttatacggctattttgtaaagttggacttggagtttatcctcttagagtcaccatgctttgtctttaattagtacgtcgaccctctcaggattttttctgatggggttactttggtacaatgccaagtatacgttttgggtgtttacttttacctcccgttccgtaagaacatttcgttgttttacctttcgatctttcagttcacctttacgttaaaaagagttgatgactccgtcggcgtccgttaccactattgaactgaggatttcaccataacatgtcacttctacatctatatctttggggtctgtgagtataaagaatgtacgggtgataattccttccattgagtgctcttgattacccggttgttagatacgggttgtccggattaatgtaacgaaaatccctgttaaaataaccagattacataatgttgtcgtgcccattatacccacaagaccgcccggttcgtagcgtcaacttacgacaacatctaaacgttctgtctttgtgtctcgaaagtatggtcgaaaacgaactaaggtaaccactatcttggtccatgaaaagatacaccttagtccgacaactgtcgatactaggtctacaatcttaataacttttagtaccttgacttctacttgaaggtttaatgacgaaaggtgaccctccacactaattatgtctctgagaatggttccattttggattttgtccagtccttttacctaccctttttctacgatgtcttaaaagtctatttttactttattctcaacctttattaaaacggtacctttagttagatttacggttggacacctctttaaaggacatgaggttgtatcgcgacataaacgggctgttcgatttcatgtcaggaaggttgcatttttaaagactattgggtttgtggatgctgatgtacttgttcgctcaccaccgagggcccgatcacctgacgatgtaattggaacctcgtgcgaccagggaactgatatacctgttgcagttgggtaaattggtggtggcgttacgaccggacgcgatggcgagttacaacgacccgttaccagcgatacacgggaaggtgtaggtccacggagtcttcaagaaacggtaatttttggaggaagaggacggcccgagtatgtggatgctcaccttgaagtccttcctacaattgtaccaagacgtctcgagggatcctttactggattcccaactgcctcggtcgtaattcaaactatcgtaaacggaaatgcggtggaagaaggggtaccgggtgttgtggcggaggtgcgaactccggtacgaatctttgctgtggttgctggtcaggaaattgctgatagagaggcggcggttgtacgagatgggatatgggcggttgcgatggttgcacgggtataggtaggggagggcgttgacccgccgaaaggcgccgacccggaagtgcgcggaattctgattcctttggggtagtgacccgagcccgatgctgggaataatgtggatgagaccgagatatgggatggatctaccttggaaaatggagttggtgtggaaattcttccaccggtaatggaaactgagaagacagtcgaccggaccgttactggcggacgaatgggggttgctcaaactttaattcgcgagtcaactgcccctcccaatgttgcaacgggtcacattgtactggtttctgaccaaggaccatgtttacgatcgattgatattgtaaccgatggtcccgaagatatagggtctctcgatgttcctggcgtacatgaggaagaaatctttgaaggtcgggtactcggcagtccaccacctactatgatttatgttcctgatggttgtccacccgtaggatgtggttgtgttgttgagacctaaacaaccgatggaacgggggtggtacgcgcttcctgtccggatgggacgattgaaggggataggcgaatatccgttctggcgtcaactgtcgtaatgggtctttttcaaagaaacgctagcgtgggaaaccgcgtagggtaagaggtcattgaaatacaggtacccgcgtgagtgtctggacccggttttggaagagatgcggttgaggcgggtgcgcgatctgtactgaaaactccacctagggtacctgctcgggtgggaagaaatacaaaacaaacttcagaaactgcaccaggcacacgtggtcggcgtggcgccgcagtagctttggcacatggacgcgtgcgggaagagccggccgttgcggtgttgtatttcttcgttcgttgtagttgttgtcgacggcggtacccgaggtcactcgtccttgactttcggtaacagtttctagaaccaacacccggtataaaaaacccgtggatactgttcgcgaaaggtccgaaacaaagaggtgtgttcgagcggacgcggtatcagttatgccggccagcgctctgacccccgcatgtgacctaccggaaacggaccttgggcgtgagtttttgtacgatggagaaactcgggaaaccgaaaagactggtcgctgagttcgtccaaatggtcaaactcatgctcagtgaggacgcggcatcgcggtaacgaagaagggggctggcgacatattgcgaccttttcaggtgggtttcgcatgtccccgggttgagccggcggacacctgataagacgacgtacaaagaggtgcggaaacggttgaccggggtttgagggtacctagtgttggggtggtacttggaataatggccccatgggttgaggtacgagttgtcaggggtccatgtcgggtgggacgcagcgttggtccttgtcgagatgtcgaaggacctcgcggtgagcgggatgaaggcgtcggtgtcacgcgtctaatcctcgcggtgaagaaaaacagtgaactttttgtacatttttattacatgatctctgtgaaagttatttccgtttacgaaaataaacatgtgagagcccactaataaatgggggtgggaacggcagacgcggcaaatttttagtttccccaagacggcgcgtagcgatacgcggtgaccgtccctgtgcaacgctatgaccacaaatcacgaggtgaatttgagtccgtgttggtaggcgccgtcgagccacttcaaaagtgaggtgtccgacgcgtggtagtggttgcgcaaatcgtccagcccgcggctatagaacttcagcgtcaaccccggaggcgggacgcgcgcgctcaacgctatgtgtcccaacgtcgtgaccttgtgatagtcgcggcccaccacgtgcgaccggtcgtgcgagaacagcctctagtctaggcgcaggtccaggaggcgcaacgagtcccgcttgcctcagttgaaaccatcgacggaagggtttttcccgcgcacgggtccgaaactcaacgtgagcgtggcatcaccgtagttttccactggcacgggccagacccgcaatcctatgtcgcggacgtattttcggaactagacgaattttcggtggactcggaaacgcggaagtctcttcttgtacggcgttctgaacggccttttgactaaccggcctgtccggcgcagcacgtgcgtcgtggaacgcagccacaacctctagacgtggtgtaaagccggggtggccaagaagtgctagaaccggaacgatctgacgaggaagtcgcgcgcgacgggcaaaagcgagcagtgtaggtaaagttagtgcacgaggaataaatagtattacgaaggcacatctgtgaattcgagcggaagctagagtcgcgtcgccacgtcggtgttgcgcgtcgggcacccgagcactacgaacatccagtggagacgtttgctgacgtccatgcggacgtccttagcggggtagtagcagtgtttccagaacaacgaccacttccagtcgacgttgggcgccacgaggagcaagtcggtccagaacgtatgccggcggtctcgaaggtgaaccagtccgtcatcaaacttcaagcggaaatctagcaataggtgcaccatgaacaggtagtcgcgcgcgcgtcggaggtacgggaagagggtgcgtctgtgctagccgtgtgagtcgcccaagtagtggcattaaagtgaaaggcgaagcgacccgagaaggagaaggagaacgcaggcgtatggtgcgcggtgacccagcagaagtaagtcggcggcgtgacacgcgaatggaggaaacggtacgaactaatcgtggccacccaacgactttgggtggtaaacatcgcggtgtagaagagaaagaaggagcgacaggtgctaatggagaccactaccgcccgcgagcccgaaccctcttcccgcgaagaaaaagaagaacccgcgttaccggtttaggcggcggctccagctaccggcgcccgacccacacgcgccgtggtcgcgcagaacactactcagaaggagcaggagcctgagctatgcggcggagtaggcgaaaaaacccccgcgggcccctccgccgccgctgcccctgcccctgctgtgcaggaggtaccaaccccctgcagcgcggcgtggcgcaggcgcgagcccccaccaaagcgcgacgaggagaagggctgaccggtaaaggaagaggatatccgtctttttctagtacctcagtcagctcttcttcctgtcggattggcgggggagactcaagcggtggtggcggaggtggctacggcggttgcgcggatggtggaaggggcagctccgtgggggcgaactcctcctccttcactaatagctcgtcctgggtccaaaacattcgcttctgctgctcctggcgagtcatggttgtctcctatttttcgttctggtcctgttgcgtctccgtttgctccttgttcagcccgcccccctgctttccgtaccgctgatggatctacaccctctgctgcacgacaacttcgtagacgtcgcggtcacgcggtaatagacgctgcgcaacgttctcgcgtcgctacacggggagcggtatcgcctacagtcggaacggatgcttgcggtggataagagtggcgcgcatggggggtttgcggttcttttgccgtgtacgctcgggttgggcgcggagttgaagatggggcataaacggcacggtctccacgaacggtggatagtgtagaaaaaggttttgacgttctatggggataggacggcacggttggcgtcggctcgcctgttcgtcgaccggaacgccgtcccgcgacagtatggactatagcggagcgagttgcttcacggtttttagaaactcccagaacctgcgctgctcttcgcgcgccgtttgcgagacgttgtccttttgtcgcttttactttcagtgagacctcacaaccaccttgagctcccactgttgcgcgcggatcggcatgattttgcgtcgtagctccagtgggtgaaacggatgggccgtgaattggatggggggttccagtactcgtgtcagtactcactcgactagcacgcggcacgcgtcggggacctctccctacgtttaaacgttcttgtttgtctcctcccggatgggcgtcaaccgctgctcgtcgatcgcgcgaccgaagtttgcgcgctcggacggctgaacctcctcgctgcgtttgattactaccggcgtcacgagcaatggcacctcgaactcacgtacgtcgccaagaaacgactgggcctctacgtcgcgttcgatctcctttgtaacgtgatgtggaaagctgtcccgatgcatgcggtccggacgttctagaggttgcacctcgagacgttggaccagaggatggaaccttaaaacgtgcttttggcggaacccgttttgcacgaagtaaggtgcgagttcccgctccgcgcggcgctgatgcaggcgctgacgcaaatgaataaagatacgatgtggaccgtctgccggtacccgcaaaccgtcgtcacgaacctcctcacgttggagttcctcgacgtctttgacgatttcgttttgaacttcctggatacctgccggaagttgctcgcgaggcaccggcgcgtggaccgcctgtagtaaaaggggcttgcggacgaattttgggacgttgtcccagacggtctgaagtggtcagtttcgtacaacgtcttgaaatccttgaaataggatctcgcgagtccttagaacgggcggtggacgacacgtgaaggatcgctgaaacacgggtaattcatggcgcttacgggaggcggcgaaaccccggtgacgatggaagacgtcgatcggttgatggaacggatggtgagactgtattaccttctgcactcgccactgccagatgacctcacagtgacagcgacgttggatacgtggggcgtggcgagggaccaaacgttaagcgtcgacgaattgctttcagtttaatagccatggaaactcgacgtcccagggagcggactgcttttcaggcgccgaggccccaactttgagtgaggccccgacacctgcagccgaatggaagcgtttaaacatggactcctgatggtgcgggtgctctaatccaagatgcttctggttagggcgggcggattacgcctcgaatggcggacgcagtaatgggtcccggtgtaagaaccggttaacgttcggtagttgtttcgggcggttctcaaagacgatgctttccctgccccccaaatgaacctgggggtcaggccgctcctcgagttgggttaggggggcggcggcgtcgggatagtcgtcgtcggcgcccgggaacgaagggtcctaccgtgggtttttcttcgacgtcgacggcggcggtgggtgcctgctcctccttatgaccctgtcagtccgtctcctccaaaacctgctcctcctcctcctgtactaccttctgaccctctcggatctgctccttcgaaggctccagcttctccacagtctgctttgtggcagtgggagccagcgtaaggggagcggccgcggggtctttagccgttggccaaggtcgtaccgatgttggaggcgaggagtccgcggcggccgtgacgggcaagcggctgggttggcatctaccctgtggtgaccttggtcccggccattcaggttcgtcggcggcggcaatcgggttctcgttgttgtcgcggttccgatggcgagtaccgcgcccgtgttcttgcggtatcaacgaacgaacgttctgacacccccgttgtagaggaagcgggcggcgaaagaagagatggtagtgccgcaccggaagggggcattgtaggacgtaatgatggcagtagagatgtcgggtatgacgtggccgccgtcgccgtcgttgtcgtcgccggtgtgtcttcgtttccgctggcctatcgttctgagactgtttcgggttctttaggtgtcgccgccgtcgtcgtcctcctcctcgcgacgcagaccgcgggttgcttgggcatagctgggcgctcgaatctttgtcctaaaaagggtgagacatacgatataaagttgtctcgtccccggttcttgttctcgacttttattttttgtccagagacgctagggagtgggcgtcgacggacatagtgttttcgcttctagtcgaagccgcgtgcgaccttctgcgcctccgagagaagtcatttatgacgcgcgactgagaattcctgatcaaagcgcgggaaagagtttaaattcgcgcttttgatgcagtagaggtcgccggtgtgggccgcggtcgtggacaacagtcgcggtaatactcgttcctttaagggtgcgggatgtacacctcaatggtcggtgtttaccctgaacgccgacctcgacgggttctgatgagttgggcttatttgatgtactcgcgccctggggtgtactatagggcccagttgccttatgcgcgggtggctttggcttaagaggaccttgtccgccgataatggtggtgtggagcattattggaattaggggcatcaaccgggcgacgggaccacatggtcctttcagggcgagggtggtgacaccatgaagggtctctgcgggtccggcttcaagtctactgattgagtccccgcgtcgaacgcccgccgaaagcagtgtcccacgccagcgggcccgtcccatattgagtggactgttagtctcccgctccataagtcgagttgctgctcagccactcgaggagcgaaccagaggcaggcctgccctgtaaagtctagccgccgcggccggcgagaagtaagtgcggagcagtccgttaggattgagacgtctggagcaggagactcggcgcgagacctccgtaaccttgagacgttaaataactcctcaaacacggtagccagatgaaattggggaagagccctggagggccggtgataggcctagttaaataaggattgaaactgcgccatttcctgagccgcctgccgatgctgacttacaattcacctctccgtctcgttgacgcggactttgtggaccaggtgacagcggcggtgttcacgaaacgggcgctgaggccactcaaaacgatgaaacttaacgggctcctagtatagctcccgggccgcgtgccgcaggccgaatggcgggtccctctcgaacgggcatcggactaagccctcaaatgggtcgcgggggacgatcaactcgccctgtcccctgggacacaagagtgacactaaacgttgacaggattgggacctaatgtagttctagaaacaacggtagagacacgactcatattatttatgtctttaattttatatgaccccgaggatagcggtaggacatttgcggtggcagaagtgggcgggttcgtttggttccgcttggaatggaccatgaaaattgtagagagggagacactaaatgttgtcaaagttgggtctgcctcactcagatgctctcttggagaggctcgagtcgatgaggtagtcttttttgtggtgggaggaatggacggcccttgcatgctcacgcagtggccggcgacgtggtgtggatggcggactggcatttggtctgaaaaaggcctgtctggagttattgagacaaatggtcttgtcctccactcgaatcttttgggaatcccataatccggtttccgcgtcgatgacaccccaaatacttgttaagttcgttgagatgcccgataagattaagtccaaagagatcttagccccaaccccaataagagacagaacactaagagaaataagaatatgattgcgaagagacggattccgagcggcggacgacacacgtgtaaacgtaaataacagtcgaaaaatttgcgaccccagcggtgggttctactaatccatgtattaggatccaaatgagtgggaacgcagtcgggtgccatggtgggttttccacctaaaattcctcggtcggacattacaatgtaagcgtcgacttcgattactcacgtggtgagaatattttacgtggtgtcttgtacttttcgacgaataagcggtgtttttgttttaaccgttcatacgacaaatacgataaaccgtcggtccactgtgatgtctcatattacaatgtcaaaaggtcccattttcagtattttgaaaatacatatgaaaaggtaaaatactttacacgctgtaatggtacatgtactcgtttgtcatattcaacaccgggggtgttttaacacaccttttgtgaccgtgaaagacgacgtgacgatacgattaatgtcacgagcgaaaccagacatgggatgagatataatttatgttttcgtctgcgtcgaaataactccttttcttttacggaattaaatgattcaatgtttcgattacagtggtgattgacgaaatgagcgacgaacgttttgtttaagtttttcaatcgtaatattaatcttatcctaaatttggggggccagtaaaggacgagttatggtaaggggacttgttaactgagatacaccctatacgaggtcgcgatgttggaacttcagtccgaaggacctacagtcgtagactgaaaccggtcgtggacagggcgcctaaacaaggtcaggttgatgtcgctgggtgggattgtctctactggttgtgttggttgcgccggcggcgatggcctgaatgtagatggtgtttatgtggggttcaaagacggaaacagttattgaccctattgaacccgtacaccaccaagaggtatcgcgaatacaaacatacggaataataatacaccgagtagacgacggatttcgcgtttgcgcgggctggtgggtagatatcagggtagtaacacgatgtgggtttgttactaccttaggtatctaacctgcctgactttgtgtacaagaaaagagaatgtcatactaatttactctgtactaaggagctcaaaaatataatgactgggaacaacgcgaaaaaacacgcacgaggtgtaaccgacgccaaagagtgtagcttcatctgacgtaaggtcggaagtgtcagataaacgaaatgcctaaacagtgggagtgcgagtagacgtcggagtagtgacaccagtagcggaaataggtcacgtaactgacccagacacacgcgaaacgtatagagtctgtggtaggggtcatgtccctgtcctgatatcgactcgaagaatcttaagaaattaatactttaaatgacactgaaaagacgactaataaacgtgggatagacgcaaaacaaggggctggaggttcggagtttctgtatatagtacgtctaagtgagcatataccttataaggttcaacgatgttacttttttcgctagaaaggcttcggaccaatatacgttagtagagacaataccacaagacgtcatggtagaatcgggatcgatatatagggatggaactgtaaccgaccttgcgttatctacggtacttggtgggttgaaaggggcgcgggcgatacgaaggtgacgttgttcaacaacggccgccgaaacagggtcggttagtcggagcgggtggaagagggtgggggtgactttagtcgatgaaattagattgtcctcctctactgactgtgggatctagatctttacctgccttaataatgtctcgtcgcggacgatctttctgcgtcccgtcgccggctcgttgtcgcgtacttagttctcgaggttctgtaccaattgaacgtggtcacgttttccccatagaaaacagagcatttcgtccggtttcagtggatgctgtcattatggtggcctgtggcggaatcgatgttcaacggttggttcgcagtctttaaccaccagtaccaccctcttttcgggtaatggtattgagtcgtgagccatctttggcttccgacgtaagtgagtggaacagttcctggactcctagagacgtgggaataattctgggacacgccagagtttctagaataagggaaattgattatttttttttattatttcgtagtgaatgaattttagtcaatcgtttaaagacaggtcaaataagtcgtcgtggaggaacgggaggagggtcgagaccataacgtcgaaggaggaccgacgtttgaaagaggtgttagatttaccttacagtcaaaggaggacaaggacaggtaggcgtgggtgatagaagtacaacaacgtctacttcgcgcgttctggcagacttctatggaagttggggcacataggtatactgtgcctttggccaggaggttgacacggaaaagaatgaggagggaaacatagggggttacccaaagttctctcagggggaccccatgagagaaacgcggataggcttggagatcaatggaggttaccgtacgaacgcgagttttacccgttgccggagagagacctgctccggccgttggaatggagggttttacattggtgacactcgggtggagagtttttttggttcagtttgtatttggacctttatagacgtggggagtgtcaatggagtcttcgggattgacaccgacggcggcgtggagattaccagcgcccgttgtgtgagtggtacgttagtgtccggggcgattggcacgtgctgaggtttgaatcgtaacggtgggttcctggggagtgtcacagtcttcctttcgatcgggacgtttgtagtccgggggagtggtggtggctatcgtcatgggaatgatagtgacggagtgggggaaattgatgacggtgaccatcgaacccgtaactgaactttctcgggtaaatatgtgttttaccttttgatcctgatttcatgccccgaggaaacgtacattgtctgctggatttgtgaaactggcatcgttgaccaggtccacactgataattattatgaaggaacgtttgatttcaatgacctcggaacccaaaactaagtgttccgttatacgttgaattacatcgtcctcctgattcctaactaagagttttgtctgcggaatatgaactacaatcaataggcaaactacgagttttggttgatttagattctgatcctgtcccgggagaaaaatatttgagtcgggtgttgaacctataattgatgttgtttccggaaatgaacaaatgtcgaagtttgttaaggtttttcgaactccaattggattcgtgacggttccccaactacaaactgcgatgtcggtatcggtaattacgtcctctacccgaacttaaaccaagtggattacgtggtttgtgtttaggggagttttgtttttaaccggtaccggatcttaaactaagtttgttccgataccaaggatttgatccttgaccggaatcaaaactgtcgtgtccacggtaatgtcatcctttgtttttattactattcgattgaaacacctggtgtggtcgaggtagaggattgacatctgatttacgtctctttctacgatttgagtgaaaccagaattgttttacaccgtcagtttatgaacgatgtcaaagtcaaaaccgacaatttccgtcaaaccgaggttatagaccttgtcaagtttcacgagtagaataatattctaaactgcttttacctcacgatgatttgttaaggaaggacctgggtcttataaccttgaaatctttacctctagaatgacttccgtgtcggatatgtttgcgacaacctaaatacggattggatagtcgaataggttttagagtgccattttgacggttttcattgtaacagtcagttcaaatgaatttgcctctgttttgatttggacattgtgattggtaatgtgatttgccatgtgtcctttgtcctctgtgttgaggttcacgtatgagatacagtaaaagtaccctgaccagaccggtgttgatgtaattactttataaacggtgtaggagaatgtgaaaaagtatgtaacgggttcttatttcttagcaaacacaatacaaagttgcacaaataaaaagttaacgtcttttaaagttcagtaaaaagtaagtcatcatatcggggtggtggtgtatcgaatatgtctagtggcatggaattagtttgagtgtcttgggatcataagttggacggtggagggagggttgtgtgtctcatgtgtcaggaaagaggggccgaccggaatttttcgtagtatagtacccattgtctgtataagaatccacaatataaggtgtgccaaaggacagctcggtttgcgagtagtcactataattatttgaggggcccgtcgagtgaattcaagtacagcgacaggtcgacgactcggtgtccgacgacaggttgaacgccaacgaattgcccgccgcttcctcttcaggtgcggatgtacccccatctcagtattagcacgtagtcctatcccgccaccacgacgtcgtcgcgcgcttatttgacgacggcggcggcgaggcaggacgtccttatgttgtaccgtcaccagaggagtcgctactaagcgtggcgggcgtcgtattccgcggaacaggaggcccgtgtcgtcgcgtgggactagagtgaatttagtcgtgtcattgacgtcgtgtcgtggtgttataacaagttttagggtgtcacgttccgcgacataggtttcgagtaccgcccctggtgtcttgggtgcaccggtagtatggtgttcgcgtccatctaattcaccgctggggagtatttgtgcgacctgtatttgtaatggagaaaaccgtacaacattaagtggtggagggccatggtatatttggagactaatttgtaccgcggtaggtggtggtaggatttggtcgaccggttttggacgggcggccgatatgtgacgtcccttggccctgaccttgttactgtcacctctcgggtcctgagcattggtacctagtagtacgagcagtactatagttacaaccgtgttgtgtccgtgtgcacgtatgtgaaggagtcctaatgttcgaggagggcgcaatcttggtatagggtcccttgttgggtaaggacttagtcgcatttagggtgtgacgtcccttctggagcgtgcattgagtgcaacacgtaacagtttcacaatgtaagcccgtcgtcgcctactaggaggtcataccatcgcgcccaaagacagagttttcctccatctgctagggatgacatgcctcacgcggctctgttggctctagcacaaccagcatcacagtacggtttaccttgcggcctgcatcagtataaaggacttcgttttggtccacgcccgcactgtttgtctagacgcagaggccagagcggcgaatctagcgagacacatcatcaacatcatataggtgagagagtttcgtaggtccgcgggggaccgaagcccaagatacatttgaggaagtacgcggcgacgggactattgtaggtggtggcgtcttattcggtgtgggtcggttggatgtgtaagcaagacgctcagtgtgtgccctcctcgcccttctcgaccttcttggtacaaaaaaaaaaataaggttttctaataggttttggagttttacttctagataattcacttgcgcgaggggaggccaccgcaccagtttgagatgtcggtttcttgtctattaccgtaaacattctacaacgtgttaccgaaggttttccgtttgccgggagtgcaggttcacctgcatttccgatttgggaagtcccacttagaggagatatttgtaaggtcgtggaagttggtacgggtttattaagagtagagcggtggaagagttatatagagattcgtttagggcttataattcaggccggtaacatttttagacgaggtctcgcgggaggtggaagtcggagttcgtcgcttagtactaacgtttttaagtccaaggagtgtctggacatattctaagttttcgccttgtaattgtttttatggcgctagggcatccagggaagcgtcccggtcgacttgtattagcacgtccagacgtgcctggtcgcgccggtgaaggggcggtccttggtactgttttcttgggtgtgactaatactgtgcgtatgagcctcgatacgattggtcgcatcggggctacattcgaacaacgtacccgccgctatattttacgttccacgacgagttttttagtccgtttcggagcgcgttttttctttcgtgtagcatcagtacgagtacgtctatttccgtccattcgaggccttggtggtgtctttttctgtggtaaaaagagagtttgtacagacgcccaaagacgtatttgtgttttattttattgtttttttgtaaatttgtaatcttcggacagaatgttgtcctttttgttgggaatattcgtattctgcctgatgccggtacggccgcactggcatttttttgaccagtggcactaatttttcgtggtggctgtcgaggagccagtacaggcctcagtattacattctgagccatttgtgtagtccaactaagtgtagccagtcacgatttttcgctggctttatcgggcccccttatgtatgggcgtccgcatctctgttgtaatgtcgggggtatcctccatattgttttaattatcctctctttttgtgtatttgtggactttttgggaggacggatccgttttatcgtgggagggcgaggtcttgttgtatgtcgcgaaggtgtcgccgtcggtattgtcagtcggaatggtcattttttcttttggataatttttttgtggtgagctgtgccgtggtcgagttagtcagtgtcacattttttcccggttcacgtctcgctcatatatatcctgattttttactgcattgccaatttcaggtgttttttgtgggtcttttggcgtgcgcttggatgcgggtctttgctttcggttttttgggtgttgaaggagtttagcagtgaaggcaaaagggtgcaatgcagtgaagggtaaaattcttttgatgttaagggttgtgtatgttcaatgaggcgggattttggatgcagtgggcggggcaagggtgcggggcgcggtgcagtgtttgaggtgggggagtaatagtataaccgaagttaggttttattccatataataactactacaattaattcttaagcctagacgctgcgctccgacctaccggaaggggtaatactaagaagagcgaaggccgccgtagccctacgggcgcaacgtccggtacgacaggtccgtccatctactgctggtagtccctgtcgaagttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttgtgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagaagttcttaacctaggcttaagaatta

SEQ ID NO: 4 - DNA, 49 bp, Artificial Sequence
  Short synthetic polyA signal (SPA)
aataaaagatctttattttcattagatctgtgtgttggttttttgtgtg

SEQ ID NO: 5 - DNA, 35759 bp, Adenovirus serotype 6
  N_region (34984)...(34984): n = a, t, c or g
catcatcaataatataccttattttggattgaagccaatatgataatgagggggtggagtttgtgacgtggcgcggggcgtgggaacggggcgggtgacgtagtagtgtggcggaagtgtgatgttgtaagtgtggcggaacacatgtaagcgccggatgtggtaaaagtgacgtttttggtgtgcgccggtgtacacgggaagtgacaattttcgcgcggttttaggcggatgttgtagtaaatttgggcgtaaccaagtaatatttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgcgcagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaaattatgggcagtgggtgatagagtggtgggtttggtgtggtaattttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcctcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggtggtgagacacaagaatcgcctgctactgttgtcttccgtccgcccggcaataataccgacggaggagcaacagcaggaggaagccaggcggcggcggcggcaggagcagagcccatggaacccgagagccggcctggaccctcgggaatgaatgttgtacaggtggctgaactgtttccagaactgagacgcattttaaccattaacgaggatgggcaggggctaaagggggtaaagaaggagcggggggcttctgaggctacagaggaggctaggaatctaacttttagcttaatgaccagacaccgtcctgagtgtgttacttttcagcagattaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagattagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtgaggtttactggtcccaattttagcggtacggttttcctggccaataccaatcttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctgtttgaaaggtgtaccttgggtatcctgtctgagggtaactccagggtgcgccacaatgtggcctccgactgtggttgctttatgctagtgaaaagcgtggctgtgattaagcataacatggtgtgtggcaactgcgaggacagggcctctcagatgctgacctgctcggacggcaactgtcacttgctgaagaccattcacgtagccagccactctcgcaaggcctggccagtgtttgagcacaacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtctcatgtagttttgtatctgttttgcagcagccgccgccatgagcgccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaaccgtttccggggtaggggagatcagctgggaagaaagcaggttcctaagcagctgcgacttaccgcagccggtgggcccgtaaatcacacctattaccggctgcaactggtagttaagagagctgcagctgccgtcatccctgagcaggggggccacttcgttaagcatgtccctgacttgcatgttttccctgaccaaatccgccagaaggcgctcgccgcccagcgatagcagttcttgcaaggaagcaaagtttttcaacggtttgaggccgtccgccgtaggcatgcttttgagcgtttgaccaagcagttccaggcggtcccacagctcggtcacgtgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggttgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttaagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcactaggtgcacgcgccaaccgcggttgtgcagggtgacaaggtcaacgctggtggctacctctccgcgtaggcgctcgttggtccagcagaggcggccgcccttgcgcgagcagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctaaccatgactttgaggtactggtatttgaagtcagtgtcgtcgcatccgccctgctcccagagcaaaaagtccgtgcgctttttggaacgcgggtttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggtccgcggctaggtctcgcgcggcggtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccagttggaggagtggctgttgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagccgggtcagggcgcgggctaggtccaggtgatacctgatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgggcccccggaggtagggggggctcgggacccgccgggagagggggcaggggcacgtcggcgccgcgcgcgggcaggagctggtgctgcgcgcggaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggagatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgccgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccgtgcggcagggatacggcgctaacgatgcatctcaacaattgttgtgtaggtactccgccaccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcgtccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactggtatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcacggacgcggttccagatgttgcgcagcggcaaaaagtgctccatggtcgggacgctctggccggtcaggcgcgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggatgctgcgctagcttttttggccactggccgcgcgcggcgtaagcggttaggctggaaagcgaaagcattaagtggctcgctccctgtagccggagggttattttccaagggttgagtcgcgggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccggacccggcactacttggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccatgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagctttaacaaccacgtgcgcacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacattctgcagagcatagtggtgcaggagcgcagcttgagcctggctgacaaggtggccgccattaactattccatgctcagtctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaaccctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgatgaggccggcctggtctacgacgcgctgcttcagcgcgtggctcgttacaacagcagcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtatcagtccgggccagactattttttccagaccagtagacaaggcctgcagaccgtaaacctgagccaggctttcaagaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgttagccgcgcgctggggcaggaggacacgggcagcctggaggcaaccctgaactacctgctgaccaaccggcggcaaaaaatcccctcgttgcacagtttaaacagcgaggaggagcgcattttgcgctatgtgcagcagagcgtgagccttaacctgatgcgcgacggggtaacgcccagcgtggcgctggacatgaccgcgcgcaacatggaaccgggcatgtatgcctcaaaccggccgtttatcaatcgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacaacgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaagaacctgcctccggcgtttcccaacaacgggatagagagcctagtggacaagatgagtagatggaagacgtatgcgcaggagcacagggatgtgcccggcccgcgcccgcccacccgtcgtcaaaggcacgaccgtcagcggggtctggtgtgggaggacgatgactcggcagacgacagcagcgtcttggatttgggagggagtggcaacccgtttgcacaccttcgccccaggctggggagaatgttttaaaaaaagcatgatgcaaaataaaaaactcaccaaggccatggcaccgagcgttggttttcttgtattccccttagtatgcggcgcgcggcgatgtatgaggaaggtcctcctccctcctacgagagcgtggtgagcgcggcgccagtggcggcggcgctgggttcacccttcgatgctcccctggacccgccgttcgtgcctccgcggtacctgcggcctaccggggggagaaacagcatccgttactctgagttggcacccctattcgacaccacccgtgtgtaccttgtggacaacaagtcaacggatgtggcatccctgaactaccagaacgaccacagcaactttctaaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgctcgcttactaaggacaaacaggtggagctgaaatacgagtgggtggagttcacgctgcccgagggcaactactccgagaccatgaccatagaccttatgaacaacgcgatcgtggagcactacttgaaagtgggcaggcagaacggggttctggaaagcgacatcggggtaaagtttgacacccgcaacttcagactggggtttgacccagtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgacctggagggtggtaacattcccgcactgttggatgtggacgcctaccaggcaagcttgaaagatgacaccgaacagggcgggggtggcgcaggcggcggcaacaacagtggcagcggcgcggaagagaactccaacgcggcagctgcggcaatgcagccggtggaggacatgaacgatcatgccattcgcggcgacacctttgccacacgggcggaggagaagcgcgctgaggccgaggcagcggccgaagctgccgcccccgctgcggaggctgcacaacccgaggtcgagaagcctcagaagaaaccggtgattaaacccctgacagaggacagcaagaaacgcagttacaacctaataagcaatgacagcaccttcacccagtaccgcagctggtaccttgcatacaactacggcgaccctcaggccgggatccgctcatggaccctgctttgcactcctgacgtaacctgcggctcggagcaggtatactggtcgttgcccgacatgatgcaagaccccgtgaccttccgctccacgcgccagatcagcaactttccggtggtgggcgccgagctgttgcccgtgcactccaagagcttctacaacgaccaggccgtctactcccagctcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccgccagtgtccaccgtggacgcggccattcagaccgtggtgcgcggagcccggcgctacgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgagccgctcgaaggctggccgcgggtattgtcactgtgccccccaggtccaggcgacgagcggccgccgcagcagccgcggccattagtgctatgactcagggtcgcaggggcaacgtgtactgggtgcgcgactcggttagcggcctgcgcgtgcccgtgcgcacccgccccccgcgcaactagattgcaataaaaaactacttagactcgtactgttgtatgtatccagcggcggcggcgcgcatcgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgatgaacttgacgacgaggtggaactgttgcacgcgaccgcgcccaggcgacgggtacagtggaaaggtcgacgcgtaagacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtgacactgcagcaggtgctgcccgcgcttgcaccgtccgaagaaaagcgcggcctaaagcgcgagtctggtgacttggcacccaccgtgcagctgatggtacccaagcgtcagcgactggaagatgtcttggaaaaaatgaccgtggagcctgggctggagcccgaggtccgcgtgcggccaatcaagcaggtggcaccgggactgggcgtgcagaccgtggacgttcagatacccaccaccagtagcactagtattgccactgccacagagggcatggagacacaaacgtccccggttgcctcggcggtggcagatgccgcggtgcaggcggccgctgcggccgcgtccaagacctctacggaggtgcaaacggacccgtggatgtttcgtgtttcagccccccggcgtccgcgccgttcaaggaagtacggcgccgccagcgcgctactgcccgaatatgccctacatccttccatcgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttacatgtggaaaaatcaaaataaaagtctggactctcacgctcgcttggtcctgtaactattttgtagaatggaagacatcaactttgcgtcactggccccgcgacacggctcgcgcccgttcatgggaaactggcaagatatcggcaccagcaatatgagcggtggcgccttcagctggggctcgctgtggagcggcattaaaaatttcggttccgccgttaagaactatggcagcaaagcctggaacagcagcacaggccagatgctgagggacaagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgacccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgtccgccgttgttgtaacccgtcctagccgcgcgtccctgcgccgcgccgccagcggtccgcgatcgttgcggcccgtagccagtggcaactggcaaagcacactgaacagcatcgtgggtttgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagttcgcccgcgccaccgagacgtacttcagcctgaataacaagtttagaaaccccacggtggcgcctacgcacgacgtgaccacagaccggtctcagcgtttgacgctgcggttcatccccgtggaccgcgaggatactgcgtactcgtacaaggcgcggttcaccctagctgtgggtgataaccgtgtgctagacatggcttccacgtactttgacatccgcggcgtgctggacaggggccctacttttaagccctactctggcactgcctacaacgcactggcccccaagggtgcccccaactcgtgcgagtgggaacaaaatgaaactgcacaagtggatgctcaagaacttgacgaagaggagaatgaagccaatgaagctcaggcgcgagaacaggaacaagctaagaaaacccatgtatatgcccaggctccactgtccggaataaaaataactaaagaaggtctacaaataggaactgccgacgccacagtagcaggtgccggcaaagaaattttcgcagacaaaacttttcaacctgaaccacaagtaggagaatctcaatggaacgaagcggatgccacagcagctggtggaagggttcttaaaaagacaactcccatgaaaccctgctatggctcatacgctagacccaccaattccaacggcggacagggcgttatggttgaacaaaatggtaaattggaaagtcaagtcgaaatgcaatttttttccacatccacaaatgccacaaatgaagttaacaatatacaaccaacagttgtattgtacagcgaagatgtaaacatggaaactccagatactcatctttcttataaacctaaaatgggggataaaaatgccaaagtcatgcttggacaacaagcaatgccaaacagaccaaattacattgcttttagagacaattttattggtctcatgtattacaacagcacaggtaacatgggtgtccttgctggtcaggcatcgcagttgaacgctgttgtagatttgcaagacagaaacacagagctgtcctaccagcttttgcttgattcaattggcgacagaacaagatacttttcaatgtggaatcaagctgttgacagctatgatccagatgtcagaattattgagaaccatggaactgaggatgagttgccaaattattgctttcctcttggtggaattgggattactgacacttttcaagctgttaaaacaactgctgctaacggggaccaaggcaatactacctggcaaaaagattcaacatttgcagaacgcaatgaaataggggtgggaaataactttgccatggaaattaacctgaatgccaacctatggagaaatttcctttactccaatattgcgctgtacctgccagacaagctaaaatacaaccccaccaatgtggaaatatctgacaaccccaacacctacgactacatgaacaagcgagtggtggctcctgggcttgtagactgctacattaaccttggggcgcgctggtctctggactacatggacaacgttaatccctttaaccaccaccgcaatgcgggcctgcgttaccgctccatgttgttgggaaacggccgctacgtgccctttcacattcaggtgccccaaaagttttttgccattaaaaacctcctcctcctgccaggctcatacacatatgaatggaacttcaggaaggatgttaacatggttctgcagagctctctgggaaacgaccttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctatatcccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacttttgactcttctgttagctggccgggcaacgaccgcctgcttactcccaatgagtttgagattaagcgctcagttgacggggagggctataacgtagctcagtgcaacatgacaaaggactggttcctagtgcagatgttggccaactacaatattggctaccagggcttctacattccagaaagctacaaagaccgcatgtactcgttcttcagaaacttccagcccatgagccggcaagtggtggacgatactaaatacaaagattatcagcaggttggaattatccaccagcataacaactcaggcttcgtaggctacctcgctcccaccatgcgcgagggacaagcttaccccgctaatgttccctacccactaataggcaaaaccgcggttgatagtattacccagaaaaagtttctttgcgaccgcaccctgtggcgcatccccttctccagtaactttatgtccatgggtgcgctcacagacctgggccaaaaccttctctacgcaaactccgcccacgcgctagacatgacctttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgcttcccaggctttgtttccccacacaagctcgcctgcgccatagttaacacggccggtcgcgagactgggggcgtacactggatggcctttgcctggaacccgcgctcaaaaacatgctacctctttgagccctttggcttttctgaccaacgtctcaagcaggtttaccagtttgagtacgagtcactcctgcgccgtagcgccattgcctcttcccccgaccgctgtataacgctggaaaagtccacccaaagcgtgcaggggcccaactcggccgcctgtggcctattctgctgcatgtttctccacgcctttgccaactggccccaaactcccatggatcacaaccccaccatgaaccttattaccggggtacccaactccatgcttaacagtccccaggtacagcccaccctgcgccgcaaccaggaacagctctacagcttcctggagcgccactcgccctacttccgcagccacagtgcgcaaattaggagcgccacttctttttgtcacttgaaaaacatgtaaaaataatgtactaggagacactttcaataaaggcaaatgtttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttacagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccagtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcatccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgagacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaagatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaagcgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctgtgctacacctggcaaacggccatgggcgtgtggcagcagtgcctggaggagcgcaacctgaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcacaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccattaacaaagcccgccaagagtttctgctacgaaagggacggggggtttacttggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaggaagcttccgaggccgaagaggtgtcagacgaaacaccgtcaccctcggtcgcattcccctcgccggcgccccagaaatcggcaaccgttcccagcattgctacaacctccgctcctcaggcgccgccggcactgcccgttcgccgacccaaccgtagatgggacaccactggaaccagggccggtaagtctaagcagccgccgccgttagcccaagagcaacaacagcgccaaggctaccgctcgtggcgcgtgcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcaacagcagcggccacgcagaagcaaaggcgaccggatagcaagactctgacaaagcccaagaaatccacagcggcggcagcagcaggaggaggagcactgcgtctggcgcccaacgaacccgtatcgacccgcgagcttagaaacaggatttttcccactctgtatgctatatttcaacagagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgaaaatcagagggcgaggtattcagctcaacgacgagtcggtgagctcctctcttggtctccgtccggacgggacatttcagatcggcggcgctggccgctcttcatttacgccccgtcaggcgatcctaactctgcagacctcgtcctcggagccgcgctccggaggcattggaactctacaatttattgaggagttcgtgccttcggtttacttcaaccccttttctggacctcccggccactacccggaccagtttattcccaactttgacgcggtaaaagactcggcggacggctacgactgaatgaccagtggagaggcagagcaactgcgcctgacacacctcgaccactgccgccgccacaagtgctttgcccgcggctccggtgagttttgttactttgaattgcccgaagagcatatcgagggcccggcgcacggcgtccggctcaccacccaggtagagcttacacgtagcctgattcgggagtttaccaagcgccccctgctagtggagcgggagcggggtccctgtgttctgaccgtggtttgcaactgtcctaaccctggattacatcaagatctttgttgtcatctctgtgctgagtataataaatacagaaattagaatctactggggctcctgtcgccatcctgtgaacgccaccgtttttacccacccaaagcagaccaaagcaaacctcacctccggtttgcacaagcgggccaataagtaccttacctggtactttaacggctcttcatttgtaatttacaacagtttccagcgagacgaagtaagtttgccacacaaccttctcggcttcaactacaccgtcaagaaaaacaccaccaccaccctcctcacctgccgggaacgtacgagtgcgtcaccggttgctgcgcccacacctacagcctgagcgtaaccagacattactcccattttcccaaaacaggaggtgagctcaactcccggaactcaggtcaaaaaagcattttgcggggtgctgggattttttaattaagtatatgagcaattcaagtaactctacaagcttgtctaatttttctggaattggggtcggggttatccttactcttgtaattctgtttattcttatactagcacttctgtgccttagggttgccgcctgctgcacgcacgtttgtacctattgtcagctttttaaacgctgggggcgacatccaagatgaggtacatgattttaggcttgctcgcccttgcggcagtctgcagcgctgccaaaaaggttgagtttaaggaaccagcttgcaatgttacatttaaatcagaagctaatgaatgcactactcttataaaatgcaccacagaacatgaaaagcttattattcgccacaaagacaaaattggcaagtatgctgtatatgctatttggcagccaggtgacactaacgactataatgtcacagtcttccaaggtgaaaatcgtaaaacttttatgtataaatttccattttatgaaatgtgcgatattaccatgtacatgagcaaacagtacaagttgtggcccccacaaaagtgtttagagaacactggcaccttttgttccaccgctctgcttattacagcgcttgctttggtatgtaccttactttatctcaaatacaaaagcagacgcagttttattgatgaaaagaaaatgccttgattttccgcttgcttgtattcccctggacaatttactctatgtgggatatgcgccaggcgggaaagattatacccacaaccttcaaatcaaactttcctggacgttagcgcctgacttctgccagcgcctgcactgcaaatttgatcaaacccagcttcagcttgcctgctccagagatgaccggctcaaccatcgcgcccacaacggactatcgcaacaccactgctaccggactaaaatctgccctaaatttaccccaagttcatgcctttgtcaatgactgggcgagcttgggcatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctcaaaccatgttctcttcttttacagtatgattaaatgagacatgattcctcgagtccttatattattgacccttgttgcgcttttctgtgcgtgctctacattggctgcggtcgctcacatcgaagtagattgcatcccacctttcacagtttacctgctttacggatttgtcacccttatcctcatctgcagcctcgtcactgtagtcatcgccttcattcagttcattgactggatttgtgtgcgcattgcgtaccttaggcaccatccgcaatacagagacaggactatagctgatcttctcagaattctttaattatgaaacggattgtcacttttgttttgctgattttctgcgccctacctgtgctttgctcccaaacctcagcgcctcccaaaagacatatttcctgcagattcactcaaatatggaacattcccagctgctacaacaaacagagcgatttgtcagaagcctggttatacgccatcatctctgtcatggttttttgcagtaccatttttgccctagccatatacccataccttgacattggttggaatgccatagatgccatgaaccaccctactttcccagcgcccaatgtcataccactgcaacaggttattgccccaatcaatcagcctcgccccccttctcccacccccactgagattagctactttaatttgacaggtggagatgactgaatctctagatctagaattggatggaattaacaccgaacagcgcctactagaaaggcgcaaggcggcgtccgagcgagaacgcctaaaacaagaagttgaagacatggttaacctgcaccagtgtaaaagaggtatcttttgtgtggtcaagcaggccaaacttacctacgaaaaaaccactaccggcaaccgccttagctacaagctacccacccagcgccaaaaactggtgcttatggtgggagaaaaacctatcaccgtcacccagcactcggcagaaacagaaggctgcctgcacttcccctatcagggtccagaggacctctgcactcttattaaaaccatgtgtggcattagagatcttattccattcaactaacaataaacacacaataaattacttacttaaaatcagtcagcaaatctttgtccagcttattcagcatcacctcctttccctcctcccaactctggtatttcagcagccttttagctgcgaactttctccaaagtctaaatgggatgtcaaattcctcatgttcttgtccctccgcacccactatcttcatattgttgcagatgaaacgcgccagaccgtctgaagacaccttcaaccctgtgtacccatatgacacggaaaccggccctccaactgtgcctttccttacccctccctttgtgtcgccaaatgggttccaagaaagtccccccggagtgctttctttgcgtctttcagaacctttggttacctcacacggcatgcttgcgctaaaaatgggcagcggcctgtccctggatcaggcaggcaaccttacatcaaatacaatcactgtttctcaaccgctaaaaaaaacaaagtccaatataactttggaaacatccgcgccccttacagtcagctcaggcgccctaaccatggccacaacttcgcctttggtggtctctgacaacactcttaccatgcaatcacaagcaccgctaaccgtgcaagactcaaaacttagcattgctaccaaagagccacttacagtgttagatggaaaactggccctgcagacatcagcccccctctctgccactgataacaacgccctcactatcactgcctcacctcctcttactactgcaaatggtagtctggctgttaccatggaaaacccactttacaacaacaatggaaaacttgggctcaaaattggcggtcctttgcaagtggccaccgactcacatgcactaacactaggtactggtcagggggttgcagttcataacaatttgctacatacaaaagttacaggcgcaatagggtttgatacatctggcaacatggaacttaaaactggagatggcctctatgtggatagcgccggtcctaaccaaaaactacatattaatctaaataccacaaaaggccttgcttttgacaacaccgcaataacaattaacgctggaaaagggttggaatttgaaacagactcctcaaacggaaatcccataaaaacaaaaattggatcaggcatacaatataataccaatggagctatggttgcaaaacttggaacaggcctcagttttgacagctccggagccataacaatgggcagcataaacaatgacagacttactctttggacaacaccagacccatccccaaattgcagaattgcttcagataaagactgcaagctaactctggcgctaacaaaatgtggcagtcaaattttgggcactgtttcagctttggcagtatcaggtaatatggcctccatcaatggaactctaagcagtgtaaacttggttcttagatttgatgacaacggagtgcttatgtcaaattcatcactggacaaacagtattggaactttagaaacggggactccactaacggtcaaccatacacttatgctgttgggtttatgccaaacctaaaagcttacccaaaaactcaaagtaaaactgcaaaaagtaatattgttagccaggtgtatcttaatggtgacaagtctaaaccattgcattttactattacgctaaatggaacagatgaaaccaaccaagtaagcaaatactcaatatcattcagttggtcctggaacagtggacaatacactaatgacaaatttgccaccaattcctataccttctcctacattgcccaggaataaagaatcgtgaacctgttgcatgttatgtttcaacgtgtttatttttcaattgcagaaaatttcaagtcatttttcattcagtagtatagccccaccaccacatagcttatactaatcaccgtaccttaatcaaactcacagaaccctagtattcaacctgccacctccctcccaacacacagagtacacagtcctttctccccggctggccttaaacagcatcatatcatgggtaacagacatattcttaggtgttatattccacacggtctcctgtcgagccaaacgctcatcagtgatgttaataaactccccgggcagctcgcttaagttcatgtcgctgtccagctgctgagccacaggctgctgtccaacttgcggttgctcaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaagtcagcacagtaactgcagcacagtaccacaatattgtttaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgtgtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgtcgcttagctcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaactgccctcacgtccaagtggacgtaaaggctaaacccttcagggtgaatctcctctataaacattccagcaccttcaaccatgcccaaataattttcatctcgccaccttatcaatatgtctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtactgctcaaaaaatcaggcaaagcctcgcgcaaaaaagcaagcacatcgtagtcatgctcatgcagataaaggcaggtaagttccggaaccaccacagaaaaagacaccatttttctctcaaacatgtctgcgggttcctgcataaacacaaaataaaataacaaaaaaaaaaaaacatttaaacattagaagcctgtnttacaacaggaaaaacaacccttataagcataagacggactacggccatgccggcgtgaccgtaaaaaaactggtcaccgtgattaaaaagcaccaccgacagttcctcggtcatgtccggagtcataatgtaagactcggtaaacacatcaggttggttaacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaccacccagaaaaccgcacgcgaacctacgcccagaaacgaaagccaaaaaacccacaacttcctcaaatcttcacttccgttttcccacgatacgtcacttcccattttaaaaaaaaactacaattcccaatacatgcaagttactccgccctaaaacctacgtcacccgccccgttcccacgccccgcgccacgtcacaaactccaccccctcattatcatattggcttcaatccaaaataaggtatattattgatgatg